(19) United States
(12) Patent Application Publication
(10) Pub. No.: US 2024/0024603 A1
(43) Pub. Date: Jan. 25, 2024

(54) RESPIRATORY DEVICE CONNECTOR

(71) Applicant: Fisher & Paykel Healthcare Limited, Auckland (NZ)

(72) Inventors: Craig Karl WHITE, Auckland (NZ); Thomas Heinrich BARNES, Auckland (NZ); Bruce Gordon HOLYOAKE, Auckland (NZ); Xavier Roy Wallace SANDER, Auckland (NZ); Anthony Behram ZEMKE, Auckland (NZ); Alicia Jerram Hunter EVANS, Auckland (NZ); Matthew Jon PAYTON, Auckland (NZ); Karan DEVA, Auckland (NZ); Ivan Justus RADEMEYER, Auckland (NZ)

(21) Appl. No.: 18/245,509

(22) PCT Filed: Aug. 24, 2021
(86) PCT No.: PCT/NZ2021/050144

§ 371 Date: Mar. 15, 2023

Related U.S. Application Data
(60) Provisional application No. 63/202,739, filed on Jun. 22, 2021.
(60) Provisional application No. 63/079,651, filed on Sep. 17, 2020.

(51) Int. Cl. A61M 16/04; A61M 16/00; A61M 16/08

(52) U.S. Cl. CPC A61M 16/0463; A61M 16/0096; A61M 16/0816; A61M 2205/3334

(57) ABSTRACT

A system for providing respiratory support to a subject includes a flow source for providing a gas at a selected flow rate, an invasive respiratory device couplable with an airway of the subject, and a connector for coupling with the invasive respiratory device. The connector includes a main body having a gases port for receiving a flow of gas from the flow source, an outlet port for outflow of gases from the main body, and a device port couplable with the invasive respiratory device. The gases port includes an inlet and an outlet. The connector is configured to receive the flow of gas from the flow source via the inlet of the gases port, and to deliver a jet flow of gas through the outlet of the gases port. The system may be configurable e.g. to generate a pressure of at least about 2 cmH₂O about the device port when in use.

The present application claims priority from U.S. Provisional Patent Application No. 63/079,651 filed on 17 Sep. 2020 and from U.S. Provisional Patent Application No. 63/202,739 filed on 22 Jun. 2021, the contents of both of which are to be taken as incorporated herein by this reference.

### TECHNICAL FIELD

The present invention relates to systems for providing respiratory support to a subject that utilise a connector for coupling with an invasive respiratory device. It also relates specifically but not exclusively to a connector for coupling with an invasive respiratory device and a kit for a system for providing respiratory support to a subject.

### BACKGROUND OF INVENTION

Patients usually require a form of respiratory support during medical procedures, particularly medical procedures which involve sedation or anaesthesia. A patient can be spontaneously breathing or apnoeic during a medical procedure or a part thereof. Invasive respiratory devices (such as an endotracheal tube (ETT), laryngeal mask (LMA) etc.) are used to provide ventilator support (e.g., by providing oxygenation and pressure support) to a patient when the patient is apnoeic.

Invasive respiratory devices such as ETTs and tracheostomy tubes can also be used to provide respiratory support to patients who are spontaneously breathing. These patients may not be undergoing a medical procedure and may be in the intensive care unit (ICU).

Weaning from ventilatory support is an important part of recovery for intubated patients in the operating theatre or in the ICU. The term “weaning” refers to the process of reducing ventilatory support, ultimately resulting in a patient breathing spontaneously and being extubated (i.e., the invasive respiratory device is removed). Prior to extubation, clinicians attempt to ensure that the patient has both sufficient respiratory drive and also sufficient respiratory strength to transit safely to stable spontaneous breathing. The process is not always successful and sometimes patients are ‘weaned’ and then extubated only for the clinician to find that they are incapable of breathing spontaneously and may have to be re-intubated.

Thus, there is a need to further improve the success of the weaning and extubation process preferably before the patient is extubated, so that the chances of re-intubation are reduced and the patient is more likely to succeed in breathing spontaneously post-extubation. It would also be desirable to provide respiratory support by oxygenating the patient and clearing carbon dioxide (CO₂) during the weaning process.

There is also a need to improve oxygenation and CO₂ clearance in patients with an invasive respiratory device, who are spontaneously breathing and who may not be undergoing a medical procedure.

A reference herein to a patent document or any other matter identified as prior art, is not to be taken as an admission that the document or other matter was known or that the information it contains was part of the common general knowledge as at the priority date of any of the claims.

### SUMMARY OF INVENTION

In one aspect, the present invention provides a system for providing respiratory support to a subject, the system including: a flow source for providing a gas at a selected flow rate; an invasive respiratory device couplable with an airway of the subject; and a connector for coupling with the invasive respiratory device, the connector including a main body having: a gases port for receiving a flow of gas from the flow source, wherein the gases port includes an inlet and an outlet; an outlet port for outflow of gases from the main body; and a device port couplable with the invasive respiratory device; wherein the connector is configured to receive the flow of gas from the flow source via the inlet of the gases port, and to deliver a jet flow of gas through the outlet of the gases port, wherein the system is configured to generate a pressure of at least about 2 cmH₂O about the device port when in use.

In some embodiments, the pressure about the device port is between about 2 cm H₂O and about 20 cmH₂O. The pressure about the device port may be between about 2 cmH₂O to about 10 cmH₂O during inspiration of the subject. Preferably, the pressure about the device port is between about 2 cmH₂O and about 5 cmH₂O during inspiration of the subject. The pressure about the device port may be between about 5 cmH₂O and about 20 cmH₂O during expiration of the subject. Preferably, the pressure about the device port is between about 5 cmH₂O and about 10 cmH₂O during expiration of the subject.

In some embodiments, a pressure loss between the outlet of the gases port and the outlet port of the connector is less than about 20 cmH₂O when in use. Preferably, the pressure loss between the outlet of the gases port and the outlet port of the connector is less than about 12 cmH₂O when in use. A ratio of the pressure about the device port to the pressure loss between the outlet of the gases port and the outlet port of the connector may be in a range of more than 0 to about 1:1.

In some embodiments, a pressure loss between the device port and the outlet port of the connector is less than about 20 cmH₂O when in use.

In some embodiments, the system includes a pressure loss between the flow source and the outlet port of the connector of less than about 20 cmH₂O when in use.

In another aspect, the present invention provides a system for providing respiratory support to a subject, the system including: a flow source for providing a gas at a selected flow rate; an invasive respiratory device couplable with an airway of the subject; and a connector for coupling with the invasive respiratory device, the connector including a main body having: a gases port for receiving a flow of gas from the flow source, wherein the gases port includes an inlet and an outlet; an outlet port for outflow of gases from the main body; and a device port couplable with the invasive respiratory device; wherein the connector is configured to receive the flow of gas from the flow source via the inlet of the gases port, and to deliver a jet flow of gas through the outlet of the gases port; wherein a pressure loss between the device port and the outlet port of the connector is less than about 20 cmH₂O when in use.

In some embodiments, the system is configured to generate a pressure of at least about 2 cmH₂O about the device port when in use. The pressure about the device port may be between about 2 cmH₂O and about 20 cmH₂O. The pressure about the device port may be between about 2 cmH₂O and about 10 cmH₂O during inspiration of the subject. Preferably, the pressure about the device port is between about 2 cmH₂O and about 5 cmH₂O during inspiration of the subject. The pressure about the device port may be between about 5 cmH₂O and about 20 cmH₂O during expiration of the subject. Preferably, the pressure about the device port is between about 5 cmH₂O and about 10 cmH₂O during expiration of the subject.

In some embodiments, a pressure loss between the outlet of the gases port and the outlet port of the connector is less than about 20 cmH₂O when in use. Preferably, the pressure loss between the outlet of the gases port and the outlet port of the connector is less than about 12 cmH₂O when in use. A ratio of the pressure about the device port to the pressure loss between the outlet of the gases port and the outlet port of the connector may be in a range of more than 0 to about 1:1.

The system may also include a pressure loss between the flow source and the outlet port of the connector of less than about 20 cmH₂O when in use.

In another aspect, the present invention provides a system for providing respiratory support to a subject, the system including: a flow source for providing a gas at a selected flow rate; an invasive respiratory device couplable with an airway of the subject; and a connector for coupling with the invasive respiratory device, the connector including a main body having: a gases port for receiving a flow of gas from the flow source, wherein the gases port includes an inlet and an outlet; an outlet port for outflow of gases from the main body; and a device port couplable with the invasive respiratory device; wherein the connector is configured to receive the flow of gas from the flow source via the inlet of the gases port, and to deliver a jet flow of gas through the outlet of the gases port, wherein a pressure loss between the outlet of the gases port and the outlet port of the connector is less than about 20 cmH₂O when in use.

Preferably, the pressure loss between the outlet of the gases port and the outlet port of the connector is less than about 12 cmH₂O when in use.

In some embodiments, a pressure loss between the device port and the outlet port of the connector is less than about 20 cmH₂O when in use.

In some embodiments, the system includes a pressure loss between the flow source and the outlet port of the connector of less than about 20 cmH₂O when in use.

In some embodiments, the system is configured to generate a pressure of at least about 2 cmH₂O about the device port when in use. The pressure about the device port may be between about 2 cmH₂O and about 20 cmH₂O. The pressure about the device port may be between about 2 cmH₂O and about 10 cmH₂O during inspiration of the subject. Preferably, the pressure about the device port is between about 2 cmH₂O and about 5 cmH₂O during inspiration of the subject. The pressure about the device port may be between about 5 cmH₂O and about 20 cmH₂O during expiration of the subject. Preferably, the pressure about the device port is between about 5 cmH₂O and about 10 cmH₂O during expiration of the subject.

In some embodiments, a ratio of the pressure about the device port to the pressure loss between the outlet of the gases port and the outlet port of the connector is in a range of more than 0 to about 1:1.

In some embodiments of the above systems disclosed herein, the flow source is configured to provide a continuous flow of the gas at the selected flow rate. The selected flow rate may include a fixed flow rate or a variable flow rate. The selected flow rate may be in a range of about 10 L/min to about 120 L/min. The selected flow rate may be in a range of about 20 L/min to about 90 L/min. The selected flow rate may be in a range of about 20 L/min to about 70 L/min. The selected flow rate may be in a range of about 40 L/min to about 70 L/min. In other embodiments of the above systems disclosed herein, the selected flow rate is in a range of about 0.5 L/min to about 25 L/min.

In some embodiments of the above systems disclosed herein, the systems further include a filter couplable with the outlet port of the connector for filtering the gases from the main body. The filter may be non-removable and/or integral with the outlet port. Alternatively, the filter may be removably couplable with the outlet port of the connector.

In some embodiments of the above systems disclosed herein, the connector further includes a filter couplable with the outlet port of the connector for filtering the gases from the main body. The filter may be non-removable and/or integral with the outlet port. The filter may be one of a radial filter or a receptacle filter. Alternatively, the filter may be removably couplable with the outlet port of the connector.

In some embodiments of the above systems disclosed herein, the connector further includes one or more gas sampling ports for sampling one or more characteristics of the gases in the main body. The one or more characteristics of the gases may include pressure, flow rate, concentration, gas constituents, temperature, humidity, contaminants, aerosols and/or pathogens. The one or more gas sampling ports may be located on one or both of the outlet port and the main body of the connector.

In some embodiments of the above systems disclosed herein, the jet flow of gas delivered through the outlet of the gases port has a velocity is in a range of about 5 m/s to about 60 m/s. The outlet of the gases port may have a hydraulic diameter in a range of about 2 mm to about 10 mm. The hydraulic diameter may be in a range of about 5 mm to about 8 mm.

In some embodiments of the above systems disclosed herein, a distance from the outlet of the gases port to a distal end portion of the invasive respiratory device when coupled to the device port is in a range of about 0 mm to about 60 mm. Preferably, the distance is in a range of about 10 mm to about 30 mm.

In some embodiments of the above systems disclosed herein, the outlet of the gases port has a cross-sectional area in a range of about 10 mm² to about 60 mm². Preferably, the cross-sectional area is in a range of about 19 mm² to about 50 mm². A ratio of the cross-sectional area of the outlet of the gases port to the distance from the outlet of the gases port to the distal end portion of the invasive respiratory device may be between about 1:1 and about 1:10.

In some embodiments of the above systems disclosed herein, the connector further includes an expiratory flow path defined between the device port and the outlet port, and wherein the expiratory flow path has a minimum cross-sectional area of at least about 25 mm². The minimum cross-sectional area may be at least about 30 mm². The minimum cross-sectional area may be at least about 35 mm².

In some embodiments of the above systems disclosed herein, the minimum cross-sectional area of the expiratory flow path is greater than a cross-sectional area of the outlet of the gases port. A ratio of the minimum cross-sectional area of the expiratory flow path to the cross-sectional area of the outlet of the gases port may be between about 2:1 and about 3:1.

In some embodiments of the above systems disclosed herein, the outlet of the gases port is disposed between the inlet of the gases port and a distal end portion of the invasive respiratory device when coupled to the device port. The outlet of the gases port may be disposed between the inlet of the gases port and the device port. Preferably, the outlet of the gases port is disposed between the inlet of the gases port and a distal end portion of the device port.

In some embodiments of the above systems disclosed herein, the gases port further includes a flow constriction for providing the jet flow of gas through the outlet of the gases port. The flow constriction may be disposed between the inlet of the gases port and the device port.

In some embodiments of the above systems disclosed herein, the flow constriction includes a nozzle having the outlet of the gases port through which the jet flow of gas is delivered.

In some embodiments of the above systems disclosed herein, the flow constriction includes the outlet of the gases port having a plurality of apertures through which the jet flow of gas is delivered.

In some embodiments of the above systems disclosed herein, the flow constriction includes a tapered region for constricting the flow of gas prior to exiting the outlet. An angle of a wall of the tapered region relative to a longitudinal axis of the flow constriction may be in a range of more than 0 degrees to about 45 degrees. Preferably, the angle is between about 2 degrees and about 20 degrees.

In some embodiments of the above systems disclosed herein, the connector further includes an inlet channel in fluid communication with the inlet of the gases port, and wherein the flow constriction is associated with the inlet channel. The flow constriction may be formed integrally with the inlet channel. Alternatively, the connector may be configured to receive an insert positionable within the inlet channel to provide the flow constriction.

In some embodiments of the above systems disclosed herein, the connector further includes an outlet channel in fluid communication with the outlet port. A cross-sectional area of the outlet channel may be greater than a cross-sectional area of the outlet of the gases port.

In some embodiments of the above systems disclosed herein, the inlet channel and the outlet channel are positioned adjacent to one another. The inlet channel and the outlet channel may be coaxial. Alternatively, a longitudinal axis of the inlet channel and a longitudinal axis of the outlet channel may be offset relative to each other.

In some embodiments of the above systems disclosed herein, the system further includes an interface conduit connectable between the gases port of the connector and the flow source for providing fluid communication. The interface conduit may be configured to heat the gas provided by the flow source to a selected temperature before delivery to the gases port of the connector.

In some embodiments of the above systems disclosed herein, the system further includes a humidifier configured to condition the gas provided by the flow source to a selected temperature and/or humidity.

In another aspect, the present invention provides a connector for coupling with an invasive respiratory device, the connector including a main body having: a gases port for receiving a flow of gas from a flow source at a selected flow rate, wherein the gases port includes an inlet and an outlet; an outlet port for outflow of gases from the main body; and a device port couplable with the invasive respiratory device; wherein the connector is configured to receive the flow of gas from the flow source via the inlet of the gases port, and to deliver a jet flow of gas through the outlet of the gases port, wherein the jet flow of gas delivered through the outlet of the gases port has a velocity in a range of about 5 m/s to about 60 m/s.

In some embodiments, the outlet of the gases port has a hydraulic diameter in a range of about 2 mm to about 10 mm. The hydraulic diameter may be in a range of about 5 mm to about 8 mm.

In some embodiments, the outlet of the gases port has a cross-sectional area in a range of about 10 mm² to about 60 mm². Preferably, the cross-sectional area is in a range of about 19 mm² to about 50 mm².

In some embodiments, a distance from the outlet of the gases port to a distal end portion of the invasive respiratory device when coupled to the device port is in a range of about 0 mm to about 60 mm. Preferably, the distance is in a range of about 10 mm to about 30 mm. A ratio of the cross-sectional area of the outlet of the gases port to the distance from the outlet of the gases port to the distal end portion of the invasive respiratory device may be between about 1:1 and about 1:10. The ratio may be between about 1:1 and about 1:5.

In some embodiments, the connector further includes an expiratory flow path defined between the device port and the outlet port, and wherein the expiratory flow path has a minimum cross-sectional area of at least about 25 mm². The minimum cross-sectional area may be at least about 30 mm². The minimum cross-sectional area may be at least about 35 mm².

In some embodiments, the minimum cross-sectional area of the expiratory flow path is greater than a cross-sectional area of the outlet of the gases port. A ratio of the minimum cross-sectional area of the expiratory flow path to the cross-sectional area of the outlet of the gases port may be between about 2:1 and about 3:1.

In some embodiments, the flow of gas at the selected flow rate has a velocity in a range of about 5 m/s to about 60 m/s.

In some embodiments, the gases port further includes a flow constriction for providing the jet flow of gas through the outlet of the gases port. The flow constriction may be disposed between the inlet of the gases port and the device port.

In some embodiments, the flow constriction includes a nozzle having the outlet of the gases port through which the jet flow of the gas is delivered.

In some embodiments, the flow constriction includes the outlet of the gases port having a plurality of apertures through which the jet flow of the gas is delivered.

In some embodiments, the flow constriction includes a tapered region for constricting the flow of gas prior to exiting the outlet. An angle of a wall of the tapered region relative to a longitudinal axis of the flow constriction may be in a range of more than 0 degrees to about 45 degrees. The angle may be between about 2 degrees and about 20 degrees.

In some embodiments, the gases port further includes a conditioning portion, preferably adjacent the outlet, having a substantially constant cross-sectional area for conditioning the flow of the gas prior to exiting the outlet. The conditioning portion may be located between the tapered region and the outlet of the gases port. The conditioning portion may have a length in a range of more than 0 mm to about 60 mm.

In some embodiments, the connector further includes an inlet channel in fluid communication with the inlet of the gases port, and wherein the flow constriction is associated with the inlet channel. The flow constriction may be formed integrally with the inlet channel. Alternatively, the connector may be configured to receive an insert positionable within the inlet channel to provide the flow constriction.

In some embodiments, the outlet of the gases port has a cross-sectional shape including one of oval, triangular, elliptical or circular. The outlet of the gases port may include an angled opening for directing the jet flow of gas along or towards a wall of the main body of the connector and/or a wall of the invasive respiratory device when coupled to the device port. The angled opening may be relative to a transverse axis of the flow constriction.

In some embodiments, the connector further includes at least one locating feature configured to maintain a desired distance between the outlet of the gases port and a distal end portion of the invasive respiratory device when coupled to the device port. The at least one locating feature may be positioned on the device port and/or the main body of the connector. The at least one locating feature may include an engagement structure for releasably coupling with the invasive respiratory device or an adapter connected to the invasive respiratory device.

In another aspect, a connector for coupling with an invasive respiratory device is disclosed herein, the connector including a main body having: a gases port for receiving a flow of gas from a flow source at a selected flow rate, wherein the gases port includes an inlet and an outlet; an outlet port for outflow of gases from the main body; and a device port couplable with the invasive respiratory device; wherein the connector is configured to receive the flow of gas from the flow source via the inlet of the gases port, and to deliver a jet flow of gas through the outlet of the gases port; and wherein the connector further includes an expiratory flow path defined between the device port and the outlet port, wherein the expiratory flow path has a minimum cross-sectional area of at least about 25 mm².

The minimum cross-sectional area may be at least about 30 mm². The minimum cross-sectional area may be at least about 35 mm².

In some embodiments, the minimum cross-sectional area of the expiratory flow path is greater than a cross-sectional area of the outlet of the gases port. A ratio of the minimum cross-sectional area of the expiratory flow path to the cross-sectional area of the outlet of the gases port may be between about 2:1 and about 3:1.

In some embodiments, the gases port further includes a flow constriction for providing the jet flow of gas through the outlet of the gases port. The flow constriction may be located between the inlet of the gases port and the device port such that it does not obstruct the expiratory flow path. The connector may further include an inspiratory flow path defined between the inlet of the gases port and the device port, wherein the flow constriction is disposed in the inspiratory flow path.

In some embodiments, the connector further includes an inlet channel and an outlet channel in flow communication with the inlet of the gases port and the outlet port, respectively, the flow constriction being associated with the inlet channel. A cross-sectional area of the outlet channel may be greater than a cross-sectional area of the outlet of the gases port. The inlet channel and the outlet channel may be positioned adjacent to one another. The inlet channel and the outlet channel may be coaxial. Alternatively, a longitudinal axis of the inlet channel and a longitudinal axis of the outlet channel may be offset relative to each other.

In some embodiments, the flow constriction includes a nozzle having the outlet of the gases port through which the jet flow of gas is delivered.

In some embodiments, the flow constriction includes the outlet of the gases port having a plurality of apertures through which the jet flow of gas is delivered.

In some embodiments, the flow constriction includes a tapered region for constricting the flow of gas prior to exiting the outlet. An angle of a wall of the tapered region relative to a longitudinal axis of the flow constriction may be in a range of more than 0 degrees to about 45 degrees. The angle may be between about 2 degrees and about 20 degrees.

In another aspect, a connector for coupling with an invasive respiratory device is disclosed herein, the connector including a main body having: a gases port having an inlet for receiving a flow of gas from a flow source at a selected flow rate; an outlet port for outflow of gases from the main body; and a device port couplable with the invasive respiratory device; wherein the connector further includes: an inlet channel in flow communication with the inlet of the gases port, wherein the connector is configured to receive an insert positionable within the inlet channel for providing an outlet; and wherein the connector is configured to receive the flow of gas from the flow source via the inlet of the gases port, and to deliver a jet flow of gas through the outlet provided by the insert.

In some embodiments, the connector is configured to receive the insert positionable within the inlet channel for providing a flow constriction, wherein the flow constriction provides the jet flow of gas through the outlet. The flow constriction may be disposed between the inlet of the gases port and the device port.

In some embodiments, the inlet channel includes at least one locating feature configured to guide positioning of the insert within the inlet channel. The at least one locating feature of the inlet channel may include one or more of: a protrusion, a groove, a rib, and/or a flange on a wall of the inlet channel.

Additionally/alternatively, the insert may include at least one locating feature to guide positioning of the insert within the inlet channel. The at least one locating feature of the insert may include a region of reduced cross-sectional area for engaging with a wall of the inlet channel.

In some embodiments, a length of the insert is selected based on a desired distance of the outlet from a distal end portion of the invasive respiratory device when coupled to the device port.

In some embodiments, the connector further includes at least one locating feature configured to maintain a desired distance between the outlet and a distal end portion of the invasive respiratory device when coupled with the device port.

In some embodiments, the connector further includes an outlet channel in flow communication with the outlet port. The outlet channel and the inlet channel may be positioned adjacent to one another. The inlet channel and the outlet channel may be coaxial. Alternatively, a longitudinal axis of the inlet channel and a longitudinal axis of the outlet channel may be offset relative to each other. In some embodiments, a cross-sectional area of the outlet channel is greater than a cross-sectional area of the outlet.

In another aspect, an insert for a connector couplable with an invasive respiratory device is disclosed herein, the connector including a main body having: a gases port including an inlet for receiving a flow of gas from a flow source at a selected flow rate; an outlet port for outflow of gases from the main body; and a device port couplable with the invasive respiratory device; wherein the connector further includes: an inlet channel in fluid communication with the inlet of the gases port, wherein the insert is configured to be positioned in the inlet channel of the connector to provide an outlet, and wherein the connector is configured to receive the flow of gas from the flow source via the inlet of the gases port, and to deliver a jet flow of gas through the outlet provided by the insert.

In some embodiments, the insert is configured to be positioned in the inlet channel of the connector to provide a flow constriction, and wherein the flow constriction provides the jet flow of gas through the outlet. The flow constriction may be disposed between the inlet of the gases port and the device port.

In some embodiments, the flow constriction is formed between the insert and a wall of the inlet channel. The insert may further include at least one locating feature to guide positioning of the insert within the inlet channel. The at least one locating feature may include a region of reduced cross-sectional area for engaging with a wall of the inlet channel.

In some embodiments, the insert includes a length selected based on a desired distance of the outlet from a distal end portion of the invasive respiratory device when coupled to the device port of the connector.

In another aspect, a connector for coupling with an invasive respiratory device is disclosed herein, the connector including a main body having: a gases port for receiving a flow of gas from a flow source at a selected flow rate, wherein the gases port includes an inlet and an outlet; an outlet port for outflow of gases from the main body; and a device port couplable with the invasive respiratory device; wherein the connector is configured to receive the flow of gas from the flow source via the inlet of the gases port, and to deliver a jet flow of gas through the outlet of the gases port, and wherein the connector is configured to change the direction of gas flow within the main body of the connector when in use.

In some embodiments, the gases port further includes a flow constriction for providing the jet flow of gas through the outlet of the gases port. The flow constriction may be disposed between the inlet of the gases port and the device port.

In some embodiments, the connector passively changes the direction of gas flow in response to inspiration and/or expiration of the subject. The connector may be configured to direct the jet flow of gas towards the device port during inspiration of the subject and towards the outlet port during expiration of the subject.

In some embodiments, the jet flow of gas is directed towards a wall of the main body of the connector opposing the outlet of the gases port. The flow constriction and/or the outlet of the gases port may be angled relative to the main body of the connector in order to direct the jet flow of gas towards the opposing wall.

In some embodiments, the opposing wall is shaped and/or positioned such that the jet flow of gas attaches to a surface of the opposing wall. The opposing wall of the main body may be curved or sloped. The opposing wall may form at least part of a wall of the outlet port.

In some embodiments, the device port and the outlet port are located at an acute angle relative to each other.

In another aspect, a connector for coupling with an invasive respiratory device is disclosed herein, the connector including a main body having: a gases port for receiving a flow of gas from a flow source at a selected flow rate, wherein the gases port includes an inlet and an outlet; an outlet port for outflow of gases from the main body; and a device port couplable with the invasive respiratory device; wherein the connector is configured to receive the flow of gas from the flow source via the inlet of the gases port, and to deliver a jet flow of gas through the outlet of the gases port, and wherein the connector further includes at least one flow altering feature for altering at least one characteristic of the jet flow of gas exiting the outlet.

In some embodiments, the gases port further includes a flow constriction for providing the jet flow of gas through the outlet of the gases port. The flow constriction may be disposed between the inlet of the gases port and the device port.

In some embodiments, the at least one flow altering feature is configured to create or increase a degree of turbulent or chaotic flow of the jet flow of gas exiting the outlet.

In some embodiments, the at least one flow altering feature is associated with the flow constriction and/or the outlet of the gases port. The at least one flow altering feature may include the flow constriction and/or the outlet of the gases port having one or both of: an internal wall with a spiral or screw-shaped structure to produce a spiral flow of the jet flow of gas exiting the outlet; and an internal wall with helical grooves to produce a rifled flow of the jet flow of gas exiting the outlet.

In some embodiments, the connector further includes an inlet channel in fluid communication with the inlet of the gases port, and wherein the at least one flow altering feature is associated with the inlet channel and/or the inlet of the gases port. The at least one flow altering feature may include the inlet channel and/or inlet of the gases port having one or both of: an internal wall with a spiral or screw-shaped structure to produce a spiral flow of the jet flow of gas exiting the outlet; and an internal wall with helical grooves to produce a rifled flow of the jet flow of gas exiting the outlet.

In some embodiments, the at least one characteristic altered includes one or more of: velocity, divergence, spread, profile and/or turbulence of the jet flow of gas exiting the outlet.

In another aspect, a connector for coupling with an invasive respiratory device is disclosed herein, the connector including a main body having: a gases port for receiving a flow of gas from a flow source at a selected flow rate, wherein the gases port includes an inlet and an outlet; an outlet port for outflow of gases from the main body; and a device port couplable with the invasive respiratory device; wherein the connector is configured to receive the flow of gas from the flow source via the inlet of the gases port, and to deliver a jet flow of gas through the outlet of the gases port, wherein the connector further includes a filter couplable with the outlet port for filtering the gases from the main body.

In some embodiments, the gases port further includes a flow constriction for providing the jet flow of gas through the outlet of the gases port. The flow constriction may be disposed between the inlet of the gases port and the device port.

In some embodiments, the filter is non-removable and/or integral with the outlet port. Alternatively, the filter may be removably couplable with the outlet port.

In some embodiments, the connector further includes an inlet channel in fluid communication with the inlet of the gases port, wherein the inlet channel is at least partly surrounded by the filter. The inlet channel may be positioned through a central axis of the filter. The connector may further include an outlet channel in fluid communication with the outlet port, wherein the outlet port is at least partly surrounded by the filter.

In some embodiments, the connector further includes a valve forming the flow constriction, wherein the valve is configured to jet flow of gas towards the device port through an outlet formed upon opening of the valve.

In some embodiments, the filter is one of a radial filter or a receptacle filter.

In another aspect, a connector for coupling with an invasive respiratory device is disclosed herein, the connector including a main body having: a gases port for receiving a flow of gas from a flow source at a selected flow rate, wherein the gases port includes an inlet and an outlet; an outlet port for outflow of gases from the main body; and a device port couplable with the invasive respiratory device; wherein the connector is configured to receive the flow of gas from the flow source via the inlet of the gases port, and to deliver a jet flow of gas through the outlet of the gases port, wherein the outlet of the gases port is offset relative to a central axis of the device port for directing the jet flow of gas along or towards a wall of the main body of the connector and/or a wall of the invasive respiratory device when coupled to the device port.

In some embodiments, the gases port further includes a flow constriction for providing the jet flow of gas through the outlet of the gases port. The flow constriction may be disposed between the inlet of the gases port and the device port.

In some embodiments, the outlet of the gases port is aligned with a wall of the main body of the connector and/or a wall of the invasive respiratory device when coupled to the device port. The outlet of the gases port may be laterally offset relative to the central axis of the device port. The outlet of the gases port may be angularly offset relative to the central axis of the device port.

In some embodiments, the gases port further includes two or more outlets that are offset relative to the central axis of the device port.

In some embodiments, the outlet of the gases port includes an angled opening relative to a transverse axis of the flow constriction for directing the jet flow of gas along or towards a wall of the main body of the connector and/or a wall of the invasive respiratory device when coupled to the device port.

In some embodiments, the connector further includes an inlet channel and an outlet channel in fluid communication with the inlet of the gases port and the outlet port, respectively, wherein the flow constriction is associated with the inlet channel, and wherein a longitudinal axis of the inlet channel and a longitudinal axis of the outlet channel are offset relative to each other.

In some embodiments, the connector further includes including at least one locating feature configured to maintain a desired distance between the outlet of the gases port and a distal end portion of the invasive respiratory device when coupled to the device port. The at least one locating feature may be positioned on the device port and/or the main body of the connector. The at least one locating feature may include an engagement structure for releasably coupling with the invasive respiratory device or an adapter connected to the invasive respiratory device.

In some embodiments, a pressure loss between the outlet of the gases port and the outlet port of the connector is less than about 20 cmH₂0 at the selected flow rate. Preferably, the pressure loss is less than about 12 cmH₂0 at the selected flow rate.

In some embodiments of the above connectors disclosed herein or the above insert disclosed herein, the outlet is disposed between the inlet of the gases port and a distal end portion of the invasive respiratory device when coupled to the device port. The outlet may be disposed between the inlet of the gases port and the device port. Preferably, the outlet is disposed between the inlet of the gases port and a distal end portion of the device port.

In some embodiments of the above connectors disclosed herein or the above insert disclosed herein, the flow constriction includes a nozzle having the outlet through which the jet flow of gas is delivered.

In some embodiments of the above connectors disclosed herein or the above insert disclosed herein, the flow constriction includes the outlet having a plurality of apertures through which the jet flow of gas is delivered.

In some embodiments of the above connectors disclosed herein or the above insert disclosed herein, the flow constriction includes a tapered region for constricting the flow of gas prior to exiting the outlet.

In some embodiments of the above connectors disclosed herein or the above insert disclosed herein, the connector may further include one or more gas sampling ports for sampling one or more characteristics of the gases in the main body. The one or more characteristics of the gases may include pressure, flow rate, concentration, gas constituents, temperature, humidity, contaminants, aerosols and/or pathogens. The one or more gas sampling ports may be located on one or both of the outlet port and the main body of the connector.

In some embodiments of the above connectors disclosed herein or the above insert disclosed herein, the flow source is configured to provide a continuous flow of the gas at the selected flow rate. The selected flow rate may include a fixed flow rate or a variable flow rate. The selected flow rate may be in a range of about 10 L/min to about 120 L/min. The selected flow rate may be in a range of about 20 L/min to about 90 L/min. The selected flow rate may be in a range of about 20 L/min to about 70 L/min. The selected flow rate may be in a range of about 40 L/min to about 70 L/min. In other embodiments of the above connectors disclosed herein or the above insert disclosed herein, the selected flow rate is in a range of about 0.5 L/min to about 25 L/min.

In another aspect, a system for providing respiratory support to a subject is disclosed herein, the system including: a flow source for providing a gas at a selected flow rate; an invasive respiratory device couplable with an airway of the subject; and the connector according to any one of the above aspects or embodiments as disclosed herein.

In some embodiments, the system is configured to generate a pressure of at least about 2 cmH₂O about the device port when in use. The pressure about the device port may be between about 2 cmH₂O and about 20 cmH₂O. The pressure about the device port may be between about 2 cmH₂O and about 10 cmH₂O during inspiration of the subject. Preferably, the pressure about the device port is between about 2 cmH₂O and about 5 cmH₂O during inspiration of the subject. The pressure about the device port may be between about 5 cmH₂O and about 20 cmH₂O during expiration of the subject. Preferably, the pressure about the device port is between about 5 cmH₂O and about 10 cmH₂O during expiration of the subject.

In some embodiments, a pressure loss between the device port and the outlet port of the connector of less than about 20 cmH₂O when in use.

In some embodiments, a pressure loss between the outlet of the gases port and the outlet port of the connector is less than about 20 cmH₂O when in use. Preferably, the pressure loss between the outlet of the gases port and the outlet port of the connector is less than about 12 cmH₂O when in use. A ratio of the pressure about the device port to the pressure loss between the outlet of the gases port and the outlet port may be in a range of more than 0 to about 1:1.

The system may include a pressure loss between the flow source and the outlet port of the connector of less than about 20 cmH₂O when in use.

In some embodiments, the flow source is configured to provide a continuous flow of the gas at the selected flow rate. The selected flow rate may include a fixed flow rate or a variable flow rate. The selected flow rate may be in a range of about 10 L/min to about 120 L/min. The selected flow rate may be in a range of about 20 L/min to about 90 L/min. The selected flow rate may be in a range of about 20 L/min to about 70 L/min. The selected flow rate may be in a range of about L/min to about 70 L/min. Alternatively, the selected flow rate may be in a range of about 0.5 L/min to about 25 L/min.

In some embodiments, the system further includes a filter couplable with the outlet port of the connector for filtering the gases from the main body.

In some embodiments, the system further includes an interface conduit connectable between the inlet of the gases port of the connector and the flow source for providing fluid communication. The interface conduit may be configured to heat the gas provided by the flow source to a selected temperature before delivery to the gases port of the connector.

In some embodiments, the system further includes a humidifier configured to condition the gas provided by the flow source to a selected temperature and/or humidity.

In another aspect, a kit for a system for providing respiratory support to a subject is disclosed herein, the kit including: the connector according to any one of the above aspects or embodiments as disclosed herein; and at least one of: a filter couplable with the outlet port of the connector; an invasive respiratory device couplable with the connector; and an adapter connectable to the device port of the connector for coupling an invasive respiratory device with the connector.

In another aspect, a kit for a system for providing respiratory support to a subject is disclosed herein, the kit including: the connector according to any one of the above aspects or embodiments as disclosed herein; and the insert according to the above aspect or any one of the embodiments as disclosed herein.

In some embodiments, the kit further includes at least one of: a filter couplable with the outlet port of the connector; an invasive respiratory device couplable with the connector; and an adapter connectable to the device port of the connector for coupling an invasive respiratory device with the connector.

In some embodiments, the kits above as disclosed herein further include an interface conduit connectable between the inlet of the gases port of the connector and the flow source for providing fluid communication. The interface conduit may be configured to heat the gas provided by the flow source to a selected temperature before delivery to the gases port of the connector.

In some embodiments, the kits above as disclosed herein further include a filter couplable between the inlet of the gases port of the connector and the flow source for filtering the gas provided by the flow source.

In some embodiments, the kits above as disclosed herein further include a humidifier configured to condition the gas provided by the flow source to a selected temperature and/or humidity.

In some embodiments, the kits above as disclosed herein further include a conduit connectable between the flow source and the humidifier, and/or a conduit connectable between the humidifier and the gases port for providing fluid communication.

In some embodiments, the kits above as disclosed herein include the humidifier having a humidification chamber and/or a humidification base unit.

In another aspect, a connector for coupling with an invasive respiratory device is disclosed herein, the connector including a main body having: a gases port for receiving a flow of gas from a flow source at a selected flow rate, wherein the gases port includes an inlet and an outlet; an outlet port for outflow of gases from the main body; a device port couplable with the invasive respiratory device; and a variable aperture for adjusting flow of gases exiting the connector through the outlet port; wherein the connector is configured to receive the flow of gas from the flow source via the inlet of the gases port, and to deliver a jet flow of gas through the outlet of the gases port, and wherein the jet flow of gas delivered through the outlet of the gases port has a velocity in a range of about 5 m/s to about 60 m/s.

In some embodiments, the connector includes a cap applied to or formed over an opening in the outlet port, the cap having a first member with a first opening and a second member with a second opening, wherein relative movement between the first member and the second member varies an amount of overlap between the first and second openings to define the variable aperture. In some embodiments, one of the first member and the second member is stationary in use, and the other of the first member and the second member is movable relative to the stationary member. Preferably, relative movement between the first member and the second member is rotational although that need not be the case and translational or other relative movements may be provided.

In some embodiments, the connector body has a first opening in a wall portion defining the outlet port, and the connector further comprises a movable collar arranged around at least part of the wall portion defining the outlet port, the collar having a second opening, wherein movement of the collar varies an amount of overlap between the first and second openings to define the variable aperture.

In some embodiments, the collar may be rotatable around the wall portion defining the outlet port. In other embodiments, the collar may be translationally moveable along the wall portion defining the outlet port.

In some embodiments, the connector comprises a connector body extension providing the variable aperture.

### BRIEF DESCRIPTION OF DRAWINGS

The invention will now be described in greater detail with reference to the accompanying drawings in which like features are represented by like numerals. It is to be understood that the embodiments shown are examples only and are not to be taken as limiting the scope of the invention as defined in the claims appended hereto.

FIG. 1 is a schematic diagram of a system for providing respiratory support to a subject, according to some embodiments of the invention.

FIG. 2 is a schematic diagram of a connector for coupling with an invasive respiratory device, according to some embodiments of the invention.

FIG. 3A is a schematic diagram of another connector for coupling with an invasive respiratory device, showing an insert for positioning in an inlet channel of the connector for providing a jet flow outlet, according to some embodiments of the invention.

FIG. 3B is a schematic diagram illustrating engagement of the insert and connector of FIG. 3A via locating features, showing the insert having a notch which engages with a protrusion on the connector, according to some embodiments of the invention.

FIG. 4 is a schematic diagram of another connector for coupling with an invasive respiratory device, showing a flow constriction in the form of a nozzle, according to some embodiments of the invention.

FIGS. 5A-C illustrate schematic diagrams of another connector for coupling with an invasive respiratory device, showing a flow constriction formed by a plurality of openings or apertures, according to some embodiments of the invention, illustrating a front view (FIG. 5A), a top view (FIG. 5B) and a side cross-sectional view (FIG. 5C).

FIG. 6 is a schematic diagram of another connector for coupling with an invasive respiratory device, showing a flow constriction in the form of a nozzle and an outlet port formed by a plurality of openings or apertures in the expiratory flow path, according to some embodiments of the invention.

FIGS. 7A-C illustrate schematic diagrams of another connector for coupling with an invasive respiratory device which is similar to FIG. 6, showing a flow constriction in the form of a nozzle and outlet port formed by a plurality of openings or apertures in the expiratory flow path, according to some embodiments of the invention, illustrating a front view (FIG. 7A), a side cross-sectional view (FIG. 7B) and a top view (FIG. 7C).

FIG. 8 is a schematic diagram of another connector for coupling with an invasive respiratory device, showing an offset outlet channel and gas sampling ports, and indicating some parameters of the connector, according to some embodiments of the invention.

FIG. 9 is a schematic diagram of another connector for coupling with an invasive respiratory device, showing an offset outlet channel, a nozzle aligned with a wall of the connector and gas sampling ports, and indicating some parameters of the connector, according to some embodiments of the invention.

FIGS. 10 and 11 show the inspiratory flow paths exiting the nozzle outlet of the connectors of FIGS. 8 and 9, respectively.

FIG. 12 is a perspective cross-sectional view of another connector for coupling with an invasive respiratory device, showing an offset outlet channel and a gases port having a constant diameter portion, according to some embodiments of the invention.

FIGS. 13 and 14 are cross-sectional views of the connector of FIG. 12 in the Z-Y plane, indicating some parameters of the connector.

FIG. 15 is a cross-sectional view of the connector of FIG. 12 in the X-Y plane, indicating some parameters of the connector.

FIG. 16 is an enlarged view of the connector of FIG. 12 showing the nozzle outlet viewed from the device port.

FIG. 17 is a simplified cross-sectional view of the connector of FIG. 12 in the Z-Y plane, showing an offset angle of the outlet channel.

FIGS. 18A and 18B are simplified cross-sectional views of the connector of FIG. 12 in the Z-Y plane, showing the nozzle angled relative to a central axis of the device port, directed along or towards a wall of the invasive respiratory device when in use (FIG. 18A) or the main body of the connector (FIG. 18B).

FIGS. 19A and 19B are cross-sectional views of the connector of FIG. 12 in the X-Y plane, showing the nozzle angled relative to a central axis of the device port, directed along or towards a wall of the invasive respiratory device when in use (left in FIG. 19A and right in FIG. 19B).

FIG. 20 is a schematic diagram of another connector for coupling with an invasive respiratory device, showing an offset outlet channel and integrally formed flow constriction, according to some embodiments of the invention.

FIG. 21 is a perspective cross-sectional view of the connector of Figure illustrating the flow constriction.

FIG. 22 is a cross-sectional view of the connector of FIG. 20 shown coupled to an adapter connected to an invasive respiratory device, according to some embodiments of the invention.

FIG. 23 is an enlarged sectional view of FIG. 22 showing the inspiratory and expiratory flow paths in the connector and adapter.

FIG. 24 is a cross-sectional view of the connector of FIG. 20 shown coupled to an adapter connected to an invasive respiratory device, and showing a filter coupled to the outlet port, according to some embodiments of the invention.

FIG. 25 is a cross-sectional view of another connector for coupling with an invasive respiratory device, showing an insert in the inlet channel to provide a jet outlet when in use, the connector being coupled to an adapter connected to an invasive respiratory device, and a filter coupled to the outlet port, according to some embodiments of the invention.

FIG. 26 is a cross-sectional view of another connector for coupling with an invasive respiratory device in the form of a wye-piece connector, showing an insert in the inlet channel to provide a jet outlet when in use, according to some embodiments of the invention.

FIG. 27 is a cross-sectional view of another connector for coupling with an invasive respiratory device, showing an insert in the inlet channel to provide a jet outlet when in use, and the connector being coupled to an adapter connected to an invasive respiratory device, according to some embodiments of the invention.

FIG. 28 is a perspective view of an insert for a connector for coupling with an invasive respiratory device, the insert including a region of reduced wall thickness for providing a flow constriction when in use, according to some embodiments of the invention.

FIG. 29 is a schematic view showing guiding of the insert of FIGS. 27 and 28 into the connector by engaging with a locating rib on a wall of the inlet channel.

FIG. 30 is an enlarged view of the locating rib and guiding surfaces of the insert shown in FIG. 29.

FIG. 31A is a schematic view of another connector for coupling with an invasive respiratory device, showing a nozzle aligned with a central axis of the distal port, and FIG. 31B is an end view showing positioning of the nozzle outlet in relation to the gases port, according to some embodiments of the invention.

FIG. 32A is a schematic view of another connector for coupling with an invasive respiratory device, showing a nozzle aligned towards a wall of an invasive respiratory device coupled to the device port in use, and FIG. 32B is an end view showing positioning of the nozzle outlet in relation to the gases port on a side away from the outlet port, according to some embodiments of the invention.

FIG. 33A is a schematic view of another connector for coupling with an invasive respiratory device, showing a nozzle aligned towards a wall of an invasive respiratory device coupled to the device port in use, and FIG. 33B is an end view showing positioning of the nozzle outlet in relation to the gases port on a side towards the outlet port, according to some embodiments of the invention.

FIG. 34A is a schematic view of another connector for coupling with an invasive respiratory device, showing a nozzle aligned with a wall of the main body of the connector, and FIG. 34B is an end view showing positioning of the nozzle outlet in relation to the gases port on a side near the outlet port, according to some embodiments of the invention.

FIG. 35A is a schematic view of another connector for coupling with an invasive respiratory device, showing a nozzle aligned with a wall of the main body of the connector, and FIG. 35B is an end view showing positioning of the nozzle outlet in relation to the gases port on a side away from outlet port, according to some embodiments of the invention.

FIG. 36A is a schematic view of another connector for coupling with an invasive respiratory device, showing a nozzle with two outlets aligned towards or along walls of an invasive respiratory device when in use, and FIGS. 36B-D are end views showing positioning of the nozzle outlets in relation to the gases port in the same orientation as FIG. 36A (see FIG. 36D) and in different orientations (see FIGS. 36B and 36C), according to some embodiments of the invention.

FIG. 37A is a schematic view of another connector for coupling with an invasive respiratory device, showing a nozzle with four outlets (two outlets omitted) aligned with towards a wall of a main body of the connector or of an invasive respiratory device when in use, and FIG. 37B is an end view showing positioning of the nozzle outlets in relation to the gases port, according to some embodiments of the invention.

FIG. 38A is a schematic view of another connector for coupling with an invasive respiratory device, showing a nozzle including a constant diameter portion, and FIG. 38B is an end view of the nozzle showing central alignment in relation to the gases port, according to some embodiments of the invention.

FIG. 39A is a schematic view of another connector for coupling with an invasive respiratory device, showing a nozzle with an outlet having an oval cross-section, and FIG. 39B is an end view of the nozzle showing central alignment in relation to the gases port, according to some embodiments of the invention.

FIGS. 40 and 41 are schematic views of connectors for coupling with an invasive respiratory device, showing a nozzle angled towards a wall of the main body of the connector, in the direction towards the outlet port (FIG. 40) and away from the outlet port (FIG. 41), according to some embodiments of the invention.

FIG. 42 is an enlarged view of a nozzle with an angled outlet relative to a transverse axis of the flow constriction, according to some embodiments of the invention.

FIGS. 43 to 47 are schematic views of connectors for coupling with an invasive respiratory device having a fluidic flip or switching mechanism, showing a nozzle directed towards an opposing wall of the main body of the connector, according to some embodiments of the invention.

FIG. 48 is a schematic view of another connector for coupling with an invasive respiratory device, showing a nozzle having a spiral structure for producing spiral flow, according to some embodiments of the invention.

FIGS. 49A-E are schematic views showing another connector for coupling with an invasive respiratory device, similar to FIG. 48, showing a nozzle having a spiral structure for producing spiral flow, according to some embodiments of the invention, illustrating a perspective view (FIG. 49A), a sectional view (FIG. 49B) through the gases port of FIG. 49A, a side view (FIG. 49C), a top cross-sectional view of the spiral structure through the gases port (FIG. 49D) and a side cross-sectional view (FIG. 49E).

FIG. 50A is an end view of another connector for coupling with an invasive respiratory device, showing a nozzle having a helical structure for producing rifling flow, and FIG. 50B is a cross-sectional view through the Section line A-A in FIG. 50A, according to some embodiments of the invention.

FIG. 51 is a schematic view of another connector for coupling with an invasive respiratory device, showing coaxial inspiratory and expiratory flow paths, according to some embodiments of the invention.

FIG. 52A is a schematic view of another connector for coupling with an invasive respiratory device, showing a nozzle positioned in the inspiratory flow path, enabling gas flow through and around the nozzle, and FIG. 52B is an end view showing the nozzle outlet centrally aligned with the gases port, according to some embodiments of the invention.

FIG. 53 is a schematic view of another connector for coupling with an invasive respiratory device, showing coaxial inspiratory and expiratory flow paths, and a radial filter on the expiratory flow path, according to some embodiments of the invention.

FIG. 54 is a cross-sectional view of the connector of FIG. 53.

FIG. 55 is a perspective sectional view of the connector of FIG. 53.

FIGS. 56 to 59 illustrate another connector for coupling with an invasive respiratory device, showing coaxial inspiratory and expiratory flow paths and a radial filter on the expiratory flow path in a perspective view (FIG. 56), perspective sectional view (FIG. 57), side view (FIG. 58) and cross-sectional view (FIG. 59), according to some embodiments of the invention.

FIGS. 60 to 63 are schematic views of connectors for coupling with an invasive respiratory device, showing a radial filter with a duckbill valve on the inspiratory path, with the expiratory path at least partly surrounded by the filter (Figure entirely surrounded by the filter (FIGS. 61 to 63), and showing a nozzle with a constant diameter portion (FIG. 63), according to some embodiments of the invention.

FIG. 64 is a schematic view of another connector for coupling with an invasive respiratory device, showing a bag or receptable filter on the expiratory path, according to some embodiments of the invention.

FIG. 65 is an enlarged view of FIG. 64 showing the nozzle.

FIGS. 66A-C are schematic illustrations showing a variable aperture which may be incorporated into a connector to allow for adjusting flow of gases exiting the connector through the outlet port, according to some embodiments of the invention.

FIGS. 67A-D are schematic illustrations showing a variable aperture which may be incorporated into a connector to allow for adjusting flow of gases exiting the connector through the outlet port, according to some embodiments of the invention.

FIG. 68A is a schematic diagram of a kit for a system for providing respiratory support to a subject, showing the inventive connector, according to some embodiments of the invention.

FIG. 68B is a schematic diagram of another kit for a system for providing respiratory support to a subject, showing the inventive connector and inventive insert, according to some embodiments of the invention.

FIGS. 69A-B to 71A-B illustrate charts showing pressure changes with increasing velocity of the jet flow (‘A’ charts) and increasing cross-sectional area of the jet outlet (‘B’ charts) for a flow rate of 20 L/min, with patient flow rates of 0 L/min (FIG. 69A-B), 15 L/min (FIG. 70A-B) and 30 L/min (FIG. 71A-B), according to some embodiments of the invention.

FIGS. 72A-B to 74A-B illustrate charts showing pressure changes with increasing velocity of the jet flow (‘A’ charts) and increasing cross-sectional area of the jet outlet (‘B’ charts) for a flow rate of 40 L/min, with patient flow rates of 0 L/min (FIG. 72A-B), 15 L/min (FIG. 73A-B) and 30 L/min (FIG. 74A-B), according to some embodiments of the invention.

FIGS. 75A-B to 77A-B illustrate charts showing pressure changes with increasing velocity of the jet flow (‘A’ charts) and increasing cross-sectional area of the jet outlet (‘B’ charts) for a flow rate of 70 L/min, with patient flow rates of 0 L/min (FIG. 75A-B), 15 L/min (FIG. 76A-B) and 30 L/min (FIG. 77A-B), according to some embodiments of the invention.

FIGS. 78A-B illustrate charts showing pressure changes with increasing velocity of the jet flow (FIG. 78A) and increasing cross-sectional area of the jet outlet (FIG. 78B) for a flow rate of 40 L/min, with a patient flow rate of 15 L/min, and including a filter on the connector outlet, according to some embodiments of the invention.

FIG. 79 illustrates a chart showing pressure changes with increasing minimum expiration area of the connector with a filter for a flow rate of 70 L/min and patient flow rate of 0 L/min, according to some embodiments of the invention.

FIG. 80 illustrates a chart showing pressure changes with increasing minimum expiration area of the connector without a filter for a flow rate of 70 L/min and patient flow rate of 0 L/min, according to some embodiments of the invention.

FIGS. 81A-C illustrate charts showing pressure changes with varying jet outlet depth for a flow rate of 40 L/min, with patient flow rates of 0 L/min (FIG. 81A), L/min (FIG. 81B) and 30 L/min (FIG. 81C), according to some embodiments of the invention.

FIGS. 82A-C illustrate charts showing pressure changes with varying jet outlet depth for a flow rate of 70 L/min, with patient flow rates of 0 L/min (FIG. 82A), L/min (FIG. 82B) and 30 L/min (FIG. 82C), according to some embodiments of the invention.

FIG. 83 illustrates a chart showing pressure changes during inspiration and expiration for a subject using the system of FIG. 1 with connectors having high expiratory resistance and low expiratory resistance, according to some embodiments of the invention.

### DETAILED DESCRIPTION

Embodiments of the invention are discussed herein by reference to the drawings which are not to scale and are intended merely to assist with explanation of the invention.

### Overview

Embodiments of the invention are generally directed to systems for providing high flow respiratory support to a patient via an invasive respiratory device, such as an endotracheal tube (ETT), a laryngeal mask (LMA) and a tracheostomy tube. The patient may be spontaneously breathing or apnoeic. Embodiments of the invention may be used in medical procedures (e.g., operating theatres), ICUs, wards, emergency departments and the like. Medical procedures should be considered broadly and can include any aspect of providing a medical procedure, including operative procedures, pre, peri and post-operative procedures, and which may or may not include the use of sedation or anaesthesia (more generally called “anaesthetic procedures”).

This respiratory support may provide a continuous flow of gases (unidirectional or positive net flow) towards the patient at high flow rates from a flow source to a spontaneously or non-spontaneously breathing patient via an ETT or other invasive respiratory device. The flow of gases is typically heated and humidified before delivery to the patient. For example, embodiments of the invention can be used during a weaning and extubation process where a patient initially starts breathing, or is attempting to breathe, through an endotracheal tube (ETT). At this point in the procedure, the system can provide a continuous flow of gases to oxygenate, clear CO₂ from the patient and/or provide pressure support to the patient. The patient can then more easily be kept in a stable condition while the clinician assesses whether they are ready to be moved to the next part of the transition for spontaneous breathing. Therefore, generation of a pressure at the top of the ETT (proximal to the patient) is potentially beneficial in patient oxygenation and/or CO₂ clearance.

The systems according to embodiments described herein may be capable of achieving desirable patient pressures for a given range of flow rates. The systems may generate a range of desirable patient pressures for providing respiratory support that are capable of achieving and/or maintaining desired airway patency for a given range of flow rates, assisting with lung recruitment, preventing or mitigating atelectasis and/or reducing the work of breathing. For spontaneously breathing patients, the flow rates delivered should meet or exceed inspiratory demand and preferably peak inspiratory demand. In certain situations where the patient is spontaneously breathing, the continuous flow of gases provided is independent of the patient's breathing, i.e., the flow of gases does not vary in synchrony with the patient's breathing. High flow respiratory support also involves delivery of respiratory support to oxygenate the patient and provide clearance of carbon dioxide.

According to embodiments of the invention, the system may include a flow source and/or a flow modulator that provides a constant or a varying flow of gases to the patient depending on the therapy, i.e. the selected flow rate that the gases port of the connector receives may be a constant or a varying flow of gases. The constant flow of gases may include a set flow rate. The varying flow of gases may include a base flow rate component and one or more oscillating flow rate components. The base flow rate component may be varying. The oscillating flow rate component may include one or more frequencies. The varying flow of gases may be independent of the patient's breathing. The flow of gases may include a constant flow of gases and a varying flow of gases, for example the flow of gases may be constant for a period of time and may be varying for another period of time. Methods and systems providing a varying flow rate are described in WO 2015/033288, WO 2016/157106 and WO 2017/187390 which are incorporated herein by reference.

Embodiments of the invention aim to effectively deliver high flow respiratory support invasively by employing a system that includes a connector configured to produce a jet flow of gas into an invasive respiratory device (such as an ETT) coupled to the connector. The system delivers inspiratory flow from a flow source, optionally via a humidifier, to the connector. The connector is configured to receive the inspiratory flow, and to deliver a jet flow of gas through an outlet of the connector and towards the invasive respiratory device and patient. An inspiratory flow path enables delivery of the inspiratory flow which is jetted through the outlet of the connector towards the invasive respiratory device and patient. Thus, inspiratory flow in this context refers to the gases which are delivered towards and/or to the patient regardless of whether the patient is breathing or apnoeic. In some embodiments, the connector includes a flow constriction for providing the jet flow of gas through the outlet of the connector. The flow constriction is preferably disposed in the inspiratory flow path to provide the jet flow of gas towards the ETT and patient. The flow constriction may include, for example, a nozzle, a tapered region for constricting the flow of gas, and/or a plurality of apertures or openings through which the jet flow of gas is delivered.

An expiratory flow path enables outflow of gases to exit through an outlet port of the connector. In some embodiments, the outlet port enables the outflow of gases to vent to atmosphere, and may include a filter. Alternatively, the outlet port may be couplable with an expiratory conduit for directing the outflow of gases to a respiratory support apparatus, such as a ventilator or anaesthesia machine. The expiratory flow in the expiratory flow path may include expiratory gases from the patient or a small amount of gases from the patient following gas exchange, both of which are returned through the invasive respiratory device and exit the outlet port. The expiratory flow may also include excess inspiratory flow, namely jetted gas flow being delivered to the patient which also exits through the outlet port. If the patient is breathing, the expiratory flow in the expiratory flow path may include expiratory gases from the patient and excess inspiratory flow. Otherwise, if the patient is apnoeic, the expiratory flow in the expiratory flow path may include excess inspiratory flow and/or a small amount of other gases from the patient following gas exchange. Thus, expiratory flow in this context refers to the gases which are returned from the patient and/or invasive respiratory device, including excess inspiratory flow, which exits through the outlet port regardless of whether the patient is breathing or apnoeic.

In this specification, invasive respiratory devices could include any device or instrument that is couplable with an airway of the subject, usually bypassing the subject's upper respiratory tract or lower respiratory airway. Invasive respiratory devices may include any device or instrument that is couplable with the lower respiratory tract or lower respiratory airway of the subject. Invasive respiratory devices include but are not limited to devices and instruments that penetrate via a patient's mouth, nose or skin to serve as an artificial airway, such as an endotracheal tube, tracheostomy tube, laryngeal mask, suspension laryngoscope, or endoscope, to name a few. It will be appreciated that these are examples only, and that the embodiments of the invention are not limited to use with endotracheal tubes or particular invasive respiratory devices described herein, and may employ other respiratory devices as would be known to a person skilled in the art.

In this specification, the terms subject and patient are used interchangeably. A subject or patient may refer to a human or an animal subject or patient.

In this specification, the terms “distal” and “proximal” are to be interpreted relative to the subject or patient. Distal refers to a feature being directed away from or further from the subject or patient. Proximal refers to a feature being directed towards or close to the subject or patient.

In this specification, the gas delivered by a flow source could include, without limitation, oxygen, carbon dioxide, nitrogen, helium, and anaesthetic agents, to name a few, or mixtures of these or other breathable gases for respiration and/or ventilation. Where reference is made to a particular gas herein, it will be appreciated that it is by way of example only and the description can apply to any gas—not just that referenced.

In this specification, it is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification, “high flow” means, without limitation, any gas flow with a flow rate that is higher than usual/normal, such as higher than the normal inspiration flow rate of a healthy patient, or higher than some other threshold flow rate that is relevant to the context. It can be provided by a non-sealing respiratory system with substantial leak happening at the entrance of the patient's airways, which is the entrance of the invasive respiratory device when the patient is intubated, the invasive respiratory device providing an artificial airway to the patient. It can also be provided with humidification to improve patient comfort, compliance and safety. “High flow” can mean any gas flow with a flow rate higher than some other threshold flow rate that is relevant to the context—for example, where providing a gas flow to a patient at a flow rate to meet inspiratory demand, that flow rate might be deemed “high flow” as it is higher than a nominal flow rate that might have otherwise been provided. “High flow” is therefore context dependent, and what constitutes “high flow” depends on many factors such as the health state of the patient, type of procedure/therapy/support being provided, the nature of the patient (big, small, adult, child) and the like. A person skilled in the art would appreciate, in a particular context what constitutes “high flow”.

But, without limitation, some indicative values of high flow can be as follows.

In some configurations, delivery of gases to a patient at a flow rate of greater than or equal to about 5 or 10 litres per minute (5 or 10 LPM or L/min).

In some configurations, delivery of gases to a patient is at a flow rate of about 5 or 10 LPM to about 150 LPM, or about 10 LPM to about 120 LPM, or about 15 LPM to about 95 LPM, or about 20 LPM to about 90 LPM, or about 20 LPM to about 70 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, according to those various embodiments and configurations described herein, a flow rate of gases supplied or provided to a connector of embodiments of the invention via a system or from a flow source, may comprise, but is not limited to, flows of at least about 5, 10, 15, 20, 30, 40, 50, 60, 70, 80, 90, 100, 110, 120, 130, 140, 150 LPM, or more, and useful ranges may be selected to be any of these values (for example, about 20 LPM to about 90 LPM, about 15 LPM to about 70 LPM, about 20 LPM to about 70 LPM, about 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM).

In configurations where the system provides a varying flow of gases comprising a base flow rate component and one or more oscillating flow rate components, the base flow rate component includes a flow rate of about 5 or 10 LPM to about 150 LPM, or about 10 LPM to about 120 LPM, or about 15 LPM to about 95 LPM, or about 20 LPM to about 90 LPM, or about 20 LPM to about 70 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, according to those various embodiments and configurations described herein, a flow rate of gases supplied or provided to a connector of embodiments of the invention via a system or from a flow source, may include, but is not limited to, flows of at least about 5, 10, 15, 20, 30, 40, 60, 70, 80, 90, 100, 110, 120, 130, 140, 150 LPM, or more, and useful ranges may be selected to be any of these values (for example, about 20 LPM to about 90 LPM, about 15 LPM to about 70 LPM, about 20 LPM to about 70 LPM, about 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM). The oscillating flow rate component includes a flow rate of about 0.05 litres/min per patient kilogram to about 0.5 litres/min per patient kilogram; and preferably about 0.12 litres/min per patient kilogram to about 0.4 litres/min per patient kilogram; and more preferably about 0.12 litres/min per patient kilogram to about 0.35 litres/min per patient kilogram.

In “high flow” the gas delivered will be chosen depending on for example the intended use of a therapy. Gases delivered may comprise a percentage of oxygen. In some configurations, the percentage of oxygen in the gases delivered may be about 15% to about 100%, 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

Flow rates for “High flow” for premature/infants/paediatrics (with body mass in the range of about 1 to about 30 kg) can be different. The flow rate can be set to about 0.4 L/min/kg to about 8 L/min/kg with a minimum of about 0.5 L/min and a maximum of about 25 L/min. For patients under 2 kg maximum flow is set to 8 L/min. In configurations where the system provides a varying flow of gases including a base flow rate component and one or more oscillating flow rate components, the flow rate of the base flow rate component can be set to about 0.4 L/min/kg to about 8 L/min/kg with a minimum of about 0.5 L/min and a maximum of about 25 L/min, and the maximum flow is set to 8 L/min for patients under 2 kg. The flow rate of the oscillating flow rate component can be set to about 0.05 L/min/kg to about 2 L/min/kg with a preferred range of about 0.1 L/min/kg to about 1 L/min/kg and another preferred range of about 0.2 L/min/kg to about 0.8 L/min/kg.

Additionally in the context of high flow support being delivered invasively, this may generate a flushing effect in the lower trachea and bronchioles such that the anatomic dead space of the upper and/or lower airways is flushed by the high incoming gas flows. This creates a reservoir of fresh gas available for each and every breath, while minimising re-breathing of carbon dioxide, nitrogen, etc.

High flow may be used as a means to promote gas exchange and/or respiratory support through the delivery of oxygen and/or other gases, and through the removal of CO₂ from the patient's airways. High flow may be particularly useful prior to, during or after a medical procedure. Further advantages of high gas flow can include increased pressure in the airways of the patient, thereby providing patency support that opens airways, the trachea, lungs/alveolar and bronchioles. The opening of these structures enhances oxygenation, and to some extent assists in removal of CO2. When humidified, the high gas flow can also prevent airways from drying out, mitigating mucociliary damage, and risks associated with airway drying, and airway obstruction, swelling and bleeding.

### System Objectives

Embodiments of the invention are directed to systems for providing high flow respiratory support being invasively delivered to a patient. Certain embodiments may achieve certain advantageous outcomes by use of an inventive connector configured to deliver a jet flow of gas into the invasive respiratory device (such as an ETT).

For simplicity, the same reference numerals have been used throughout this specification for the systems 100 according to the inventive aspects as disclosed herein, and the connectors 200 according to the inventive aspects of the invention as disclosed herein. Thus the systems 100 and connectors 200 may encompass one or more of the inventive aspects, as described in relation to the embodiments of the invention. Furthermore, it is intended that features of the systems 100 and the connectors 200 sharing the same reference numerals correspond to the same features as described in connection with embodiments of the invention.

Embodiments of the invention may provide systems that include at least one of three inventive aspects described below, although combinations of two or more of the inventive aspects is desirable. Embodiments of the invention may provide systems that include one or more of the three inventive aspects in combination with any combinations of embodiments of the inventive systems as disclosed herein. Embodiments of the invention may provide systems including an inventive connector according to any one of the aspects of the invention as disclosed herein, or combinations thereof, and/or any combinations of embodiments of the inventive connectors as disclosed herein. Embodiments of the invention may also provide connectors that include any one of the aspects of the invention as disclosed herein, or combinations thereof, and/or any combinations of embodiments of the inventive connectors as disclosed herein.

### Target Patient Pressure

Embodiments of the invention are directed to a system configured to provide respiratory support to a subject by generating a pressure within a range of desirable patient pressures for a given range of flow rates. Desirable patient pressures for providing respiratory support may include a pressure or a range of pressures that are capable of achieving and/or maintaining a patent patient airway, assisting with lung recruitment, preventing or mitigating atelectasis and/or reducing the work of breathing. The inventors have found that the lowest value of patient pressure acceptable for providing respiratory support is about 2 cmH₂O.

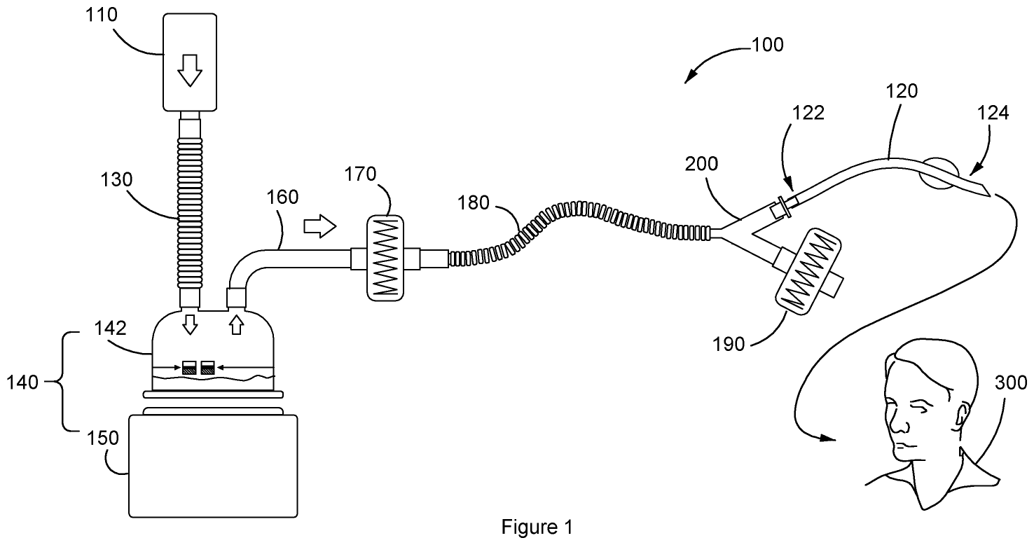

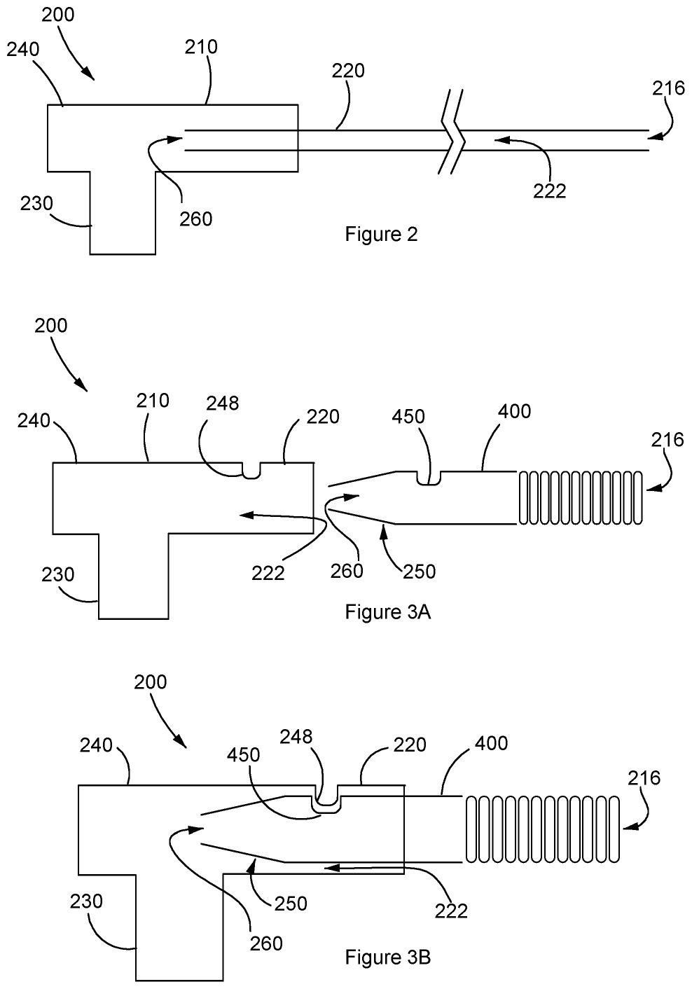

In a first inventive aspect, there is provided a system 100 for providing respiratory support to a subject 300, as illustrated in FIG. 1. The system 100 includes a flow source 110 for providing a gas at a selected flow rate, an invasive respiratory device 120 couplable with an airway of the subject 300, and a connector 200 for coupling with the invasive respiratory device 120 (see FIG. 2 for exemplary connector features). The connector 200 includes a main body 210 having a gases port 220 for receiving a flow of gas from the flow source 110, an outlet port 230 for outflow of gases from the main body 210, and a device port 240 couplable with the invasive respiratory device 120. The gases port 220 includes an inlet 216 and an outlet 260 (see also FIG. 2). The connector 200 is configured receive the flow of gas from the flow source 110 via the inlet 216 of the gases port 220, and to deliver a jet flow of gas through the outlet 260 of the gases port 220. The system 100 is configured to generate a pressure of at least about 2 cmH₂O about the device port 240 when in use.

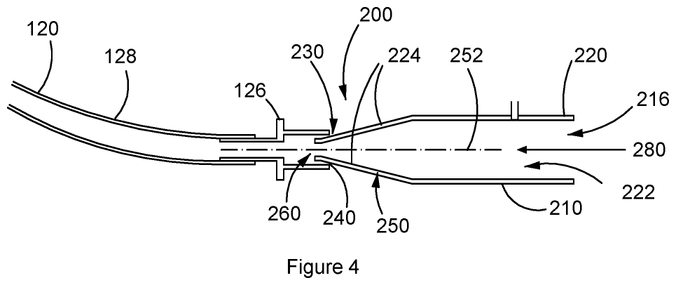

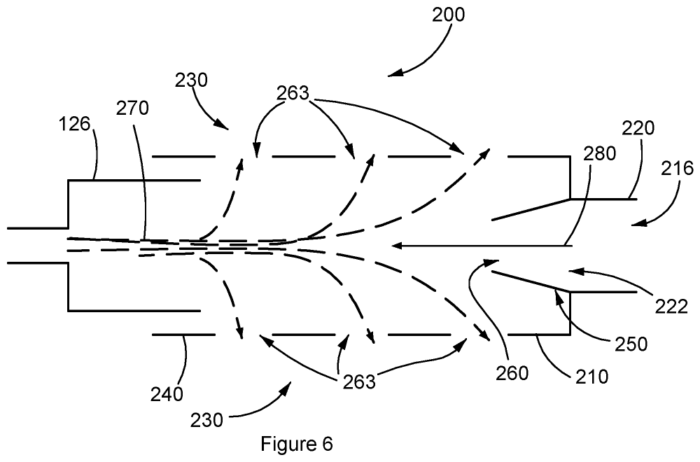

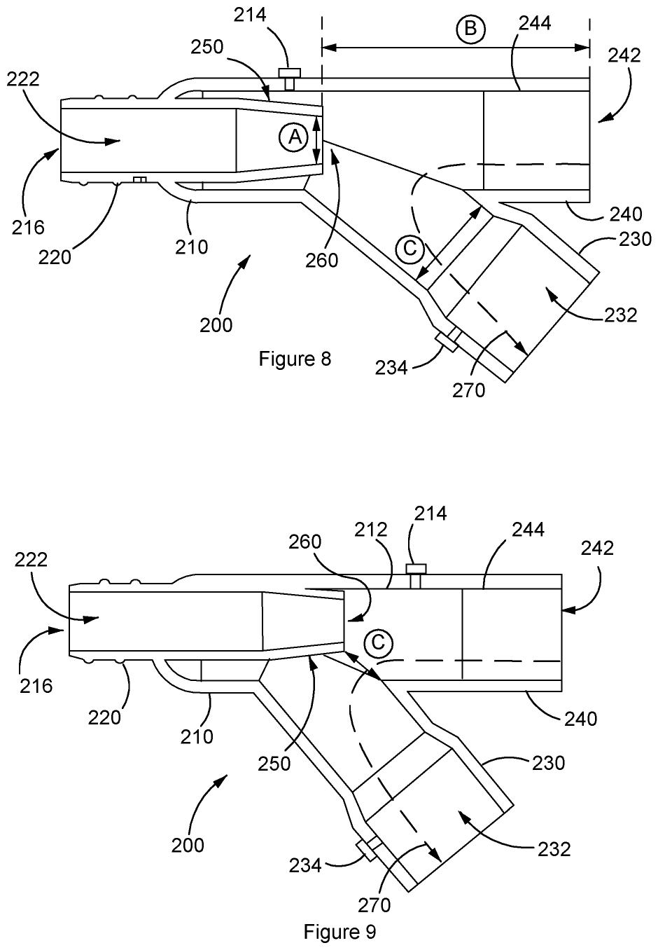

The inventors have found that there is minimal pressure loss between the device port 240 of the connector 200 and a proximal end portion 124 of the invasive respiratory device 200 to be located in the airway of the subject 300 (see FIG. 1). Thus, the pressure about the device port 240 of the connector 200 can be considered to correspond substantially to the patient pressure generated by the system 100. Notably, the pressure about the device port 240 can be measured across any part of the port, such as across the proximal opening 242 adjacent the invasive respiratory device 120 or across an internal wall 244 of the device port 240 anywhere along its length (see also FIGS. 8 and 9). The pressure about the device port 240 can also be measured across a distal end portion 122 of the invasive respiratory device 120 when coupled directly to the device port 240 (see FIG. 1), or across an adapter 126 connected to the invasive respiratory device 120 when coupled to the device port 240 (see FIGS. 4 and 6).

The inventors have also found that the highest desirable pressure about the device port 240 is about 20 cmH₂O. Above 20 cmH₂O, there could be a risk of barotrauma to the subject 300. As such, embodiments of the system 100 may be configured to generate a pressure of between about 2 cmH₂O and about 20 cmH₂O.

Furthermore, the system 100 aims to provide a target patient pressure during inspiration of between about 2 cmH₂O and about 10 cmH₂O. Preferably, the target patient pressure during inspiration is between about 2 cmH₂O and about 5 cmH₂O. The system 100 also aims to provide a target patient pressure during expiration of between about 5 cmH₂O and about 20 cmH₂O. Preferably, the target pressure during expiration is between about 5 cmH₂O and about 10 cmH₂O.

Thus, embodiments of the system 100 may be configured to generate a pressure of between about 2 cmH₂O and about 10 cmH₂O during inspiration of the subject 300, preferably between about 2 cmH₂O and about 5 cmH₂O during inspiration, and a pressure of about 5 cmH₂O and about 20 cmH₂O during expiration of the subject 300, preferably between about 5 cmH₂O and about 10 cmH₂O during expiration.

### Low Resistance to Flow (RTF)

In some embodiments, the system 100 is configured to provide respiratory support to a subject 300 by having a low resistance to expiratory flow. This involves the system 100 having an expiratory resistance to flow (RTF) of less than about 20 cmH₂O to a gases flow from the device port 240 to the outlet port 230. In some embodiments, the system 100 provides an expiratory RTF of less than about 20 cmH₂O to an expiratory flow from the device port 240 to the outlet port 230 during an expiratory phase of a spontaneously breathing patient.

In another inventive aspect, there is provided a system 100 for providing respiratory support to a subject 300 as illustrated in FIG. 1. The system 100 includes a flow source 110 for providing a gas at a selected flow rate, an invasive respiratory device 120 couplable with an airway of the subject 300, and a connector 200 for coupling with the invasive respiratory device 120. The connector 200 includes a main body 210 having a gases port 220 for receiving a flow of gas from the flow source 110, an outlet port 230 for outflow of gases from the main body 210, and a device port 240 couplable with the invasive respiratory device 120. The gases port 220 includes an inlet 216 and an outlet 260. The connector 200 is configured to receive the flow of gas from the flow source 110 via the inlet 216 of the gases port 220, and to deliver a jet flow of gas through the outlet 260 of the gases port 220. A pressure loss between the device port 240 and the outlet port 230 of the connector 200 is less than about 20 cmH₂O when in use.

The pressure loss corresponds to the expiratory resistance to flow (RTF) of the connector 200 in the system 100. This is the pressure loss across the expiratory flow path 270, which is defined between the device port 240 and the outlet port 230 of the connector 200 (see also FIGS. 8 and 9). The expiratory flow path 270 enables outflow of gases to exit through the outlet port 230 of the connector 200. If the subject 300 is breathing, the expiratory flow in the expiratory flow path 270 includes expiratory gases from the subject 300 which are returned through the invasive respiratory device 120, into the connector 200 and exit through the outlet port 230 during expiration. The expiratory flow may also include excess inspiratory flow, namely jetted gas flow being delivered to the subject 300 which also exits the outlet port 230. Otherwise, if the subject 300 is apnoeic, the expiratory flow in the expiratory flow path 270 may include excess inspiratory flow and/or a small amount of other gases from the subject 300 following gas exchange.

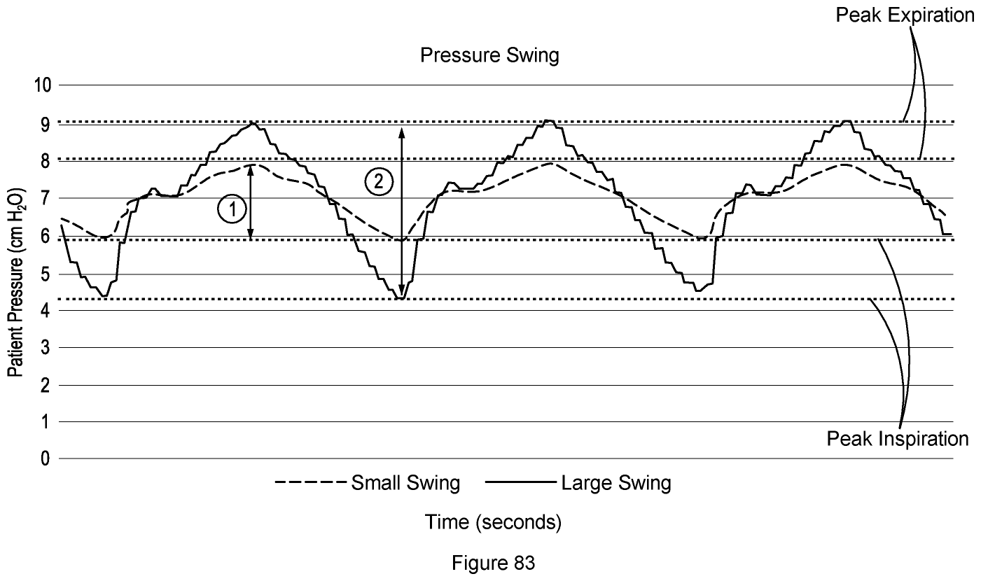

A connector 200 having a lower RTF results in less pressure loss in the system 100, increasing efficiency, and also requires less driving pressure generated by the system 100, which is discussed below. Furthermore, a lower expiratory RTF also decreases the work of breathing for the subject 300 by decreasing the lung pressure excursion required to maintain a given minute volume (i.e., flow). Some of the beneficial effects of lower RTF can be observed in relation to Example 4 and the chart of FIG. 83 as will be described below.

### Low Driving Pressure

In some embodiments, the system 100 is configured to provide respiratory support to a subject 300 by having a low driving pressure. The driving pressure is the pressure required to drive a desired flow of gas through the system 100 and preferably, to achieve a desired patient pressure and/or flow rate.

In another inventive aspect, there is provided a system 100 for providing respiratory support to a subject 300 as illustrated in FIG. 1. The system 100 includes a flow source 110 for providing a gas at a selected flow rate, an invasive respiratory device 120 couplable with an airway of the subject 300, and a connector 200 for coupling with the invasive respiratory device 120. The connector 200 includes a main body 210 having a gases port 220 for receiving a flow of gas from the flow source 110, an outlet port 230 for outflow of gases from the main body 210, and a device port 240 couplable with the invasive respiratory device 120. The gases port 220 includes an inlet 216 and an outlet 260. The connector 200 is configured to receive the flow of gas from the flow source 110 via the inlet 216 of the gases port 220, and to deliver a jet flow of gas through the outlet 260 of the gases port 220. A pressure loss between the outlet 260 of the gases port 220 and the outlet port 230 of the connector 200 is less than about 20 cmH₂O when in use.

The driving pressure corresponds to the pressure loss in the system 100. The driving pressure may include the pressure loss across the connector 200, that is the pressure loss between the outlet 260 of the gases port 220 and the outlet port 230. The pressure loss between the outlet 260 of the gases port 220 and the outlet port 230 may be less than about 20 cmH₂O when in use. Preferably, the pressure loss is less than about 12 cmH₂O when in use. The driving pressure may also include a pressure loss between the flow source 110 and the outlet port 230 of the connector 200. In that case, the pressure loss between the flow source 110 and the outlet port 230 of the connector 200 is preferably less than about 20 cmH₂O.

A lower driving pressure is desirable as the system 100 can achieve the desired patient pressures as described above with lower performance. This has an impact on the complexity of the system 100 as well as potentially the system portability. A system 100 with a lower driving pressure also has a lower risk of barotrauma for the patient 300.

In some embodiments of the above systems according to aspects of the invention, a ratio of the pressure about the device port 240 to the pressure loss between the outlet 260 of the gases port 220 and the outlet port 230 is in a range of more than 0 to about 1:1. Preferably, the ratio is in a range of about 0.3:1 to about 1:1. More preferably, the ratio is in a range of about 0.6:1 to about 1:1. The ratio may be, for example, in a range of about 0.1:1 to about 1:1, about 0.2:1 to about 1:1, about to about 1:1, about 0.4:1 to about 1:1, about 0.5:1 to about 1:1, about 0.6:1 to about 1:1, about 0.7:1 to about 1:1, about 0.8:1 to about 1:1, about 0.9:1 to about 1:1, or may be about 0.1:1, 0.2:1, 0.3:1, 0.4:1, 0.5:1, 0.6:1, 0.7:1, 0.8:1, 0.9:1 or 1:1. Ideally, the ratio is about 1:1 which represents minimal pressure loss in the system 100 such that the system 100 can be driven at a lower pressure to achieve the desired patient pressures, which preferably reduces the risk of barotrauma for the patient 300.

### Exemplary Systems for Providing Respiratory Support

FIG. 1 shows a schematic diagram of an exemplary system 100 for providing respiratory support to a subject 300. The system 100 includes a flow source 110 for providing a gas at a selected flow rate, an invasive respiratory device 120 couplable with an airway of the subject 300 and a connector 200 for coupling with the invasive respiratory device 120.

The system 100 of FIG. 1 illustrates the flow of gas from the flow source 110 to the connector 200 and onto the patient 300, in the direction of arrows as indicated. The flow source 110 may include a compressed gas source, a device that modifies the flow from a compressed gas source and/or a flow generator which generates a gas flow. The flow source 110 preferably delivers flow rates for providing high flow respiratory support being invasively delivered to a patient 300. Ideally, the flow source 110 delivers flow at high flow rates including between about 10 L/min and about 120 L/min. Preferably, the flow rates are between about 20 L/min and about 90 L/min. The flow rates may be between about 20 L/min and about 70 L/min. The flow rates may be between about 40 L/min and about 70 L/min. The range of high flows delivered to achieve sufficient patient oxygen and CO₂ clearance as well as maintain a suitable patient pressure and a desirable expiratory resistance is typically between about 20 L/min and about 70 L/min. However, this range is dependent on the patient 300 supported by the system 100, for example infants and children may not tolerate as high flow rates, and will require lower rates which are still considered as high flow for them, as defined previously in the Overview. Preferably for infants and children, the selected flow rate is in a range of about 0.5 L/min to about 25 L/min.

In some embodiments, the flow source 110 is configured to provide a continuous flow of gas at the selected flow rate. The continuous flow may be a unidirectional or positive net flow towards the patient at high flow rates. Furthermore, the selected flow rate for the flow source 110 may be a fixed flow rate or a variable (or varying) flow rate. The fixed or variable (varying) flow rate may be independent of the respiratory cycle of the patient 300. The variable or varying flow rates may be in the range of those defined previously in the Overview.

The gas flow is delivered from the flow source 110 to a conduit 130 connectable between the flow source 110 and a humidifier 140. In the exemplary system 100 as shown, the humidifier 140 includes a humidification chamber 142 and a humidification base unit 150. The conduit 130 may include a dry line for delivering dry flow of gases to the humidifier 140. The conduit 130 may be coupled to the humidification chamber 142 of the humidifier 140 as shown. In alternative embodiments, the humidifier 140 may be a single component and exclude the separate humidification chamber 142 and base unit 150 (not shown). The humidifier 140 may be configured to condition the gas provided by the flow source 110 to a selected temperature and/or humidity, for example, within the humidification chamber 142 as shown. The temperature and/or humidity selected may be dependent on the therapy being delivered and is selected to be suitable for the respiratory support to be provided, which may be tailored for human or animal subjects. A user or operator may select the desired temperature and/or humidity. Additionally/alternatively, the humidifier 140 may be configured to select the desired temperature and/or humidity by identifying a specific conduit 130 in use, e.g., by use of a sensor such as a resistor on the conduit 130.

The conditioned gas flow proceeds from the humidifier 140 (or more specifically the humidification chamber 142 as shown) through an inspiratory conduit 160 connectable between the humidifier 140 and an inlet 216 of a gases port 220 of the connector 200 for providing fluid communication (see FIG. 2). In other embodiments, the system 100 may exclude any humidifier component and the flow source 110 may be directly couplable, or couplable through an interface conduit 180, to the inlet 216 of the gases port 220 of the connector 200.

The system 100 includes an optional filter 170. The filter 170 may be positioned between the inspiratory conduit 160 and the patient interface conduit 180. Gases flowing through the inspiratory conduit 160 are passed to the patient by way of the optional filter 170, the connector 200 and the invasive respiratory device 120. The interface conduit 180 is connectable between the inlet 216 of the gases port 220 of the connector 200 and the flow source 110 for providing fluid communication. The interface conduit 180 may be located in the system 100 between the conduit 160 and/or filter 170 and the connector 200.

A filter 190 may also be optionally provided in the expiratory flow path 270 (see also FIGS. 8 and 9, and embodiments of FIGS. 53 to 65) of the connector 200, defined by the gas flow path in the connector 200 between the device port 240 and the outlet port 230. More particularly, the filter 190 may be couplable to the outlet port 230 of the connector 200 for filtering the gases from the main body 210, as shown in FIG. 1. During expiration, expiratory flow from the subject 300 passes along the expiratory flow path 270 and outflows from the main body 210 of the connector 200 via the outlet port 230 to atmosphere (see also FIGS. 8 and 9). A filter 190 may be used if the subject 300 is infectious or is provided with gases containing nebulized drugs that can be harmful to surrounding personnel or to the environment. The filter 190 preferably captures all contaminants, aerosols, pathogens, etc. in the expiratory flow. The filter 190 may be altered and tuned to impact the expiratory resistance characteristics of the system 100 (i.e., lowering expiratory RTF of the connector 200), as will be described in more detail.

In some embodiments, the filters 170 and 190 may be non-removable and/or integral with conduit 160 and connector 200, respectively. Alternatively, the filters 170 and 190 may be releasably couplable with the conduit 160 and connector 200, respectively. The filter 190 may include a radial filter, or a receptable or bag filter, as will be described in connection with FIGS. 53 to 65.

The inspiratory conduit 160 and patient interface conduit 180 may be one or more of corrugated, flexible, bendable, resistant to kink and/or heated (e.g., the conduits 160, 180 may include a heating element). In some embodiments, the inspiratory conduit 160 and/or the patient interface conduit 180 is configured to heat the gas provided by the flow source 110 to a selected temperature before delivery to the gases port 220 of the connector 200. In this embodiment, the inspiratory conduit 160 and/or the patient interface conduit 180 may include a heating element such as a heating wire. The temperature may be dependent on the therapy being delivered and is selected to be suitable for the respiratory support to be provided, which may be tailored for human or animal subjects. A user or operator may select the desired temperature. Additionally/alternatively, the conduit 180 may be a breathable tube, such as described in U.S. Pat. No. 7,493,902 which is incorporated herein by reference.

FIG. 2 is a schematic diagram of a connector 200 for coupling with an invasive respiratory device 120, according to some embodiments of the invention. The connector 200 includes a main body 210 having a gases port 220 which receives gas flow e.g., from a flow source 110 at an inlet 216 of the gases port 200, such as illustrated in the system 100 of FIG. 1. The gas flow enters the gases port 220 at the inlet 216 and travels through an inlet channel 222 in fluid communication with the inlet 216 of the gases port 220, and towards an outlet 260 of the gases port 220. The connector 200 is configured to deliver a jet flow of gas through the outlet 260. The jet flow of gas is directed in the connector main body 210 towards a device port 240 and into an invasive respiratory device 120 (e.g., an ETT as illustrated in FIG. 4) for providing respiratory support to a subject 300. The main body 210 of the connector 200 also includes an outlet port 230 for outflow of gases from the main body 210.

In the connector 200 as shown in FIG. 2, the gases port 220 includes the inlet channel 222 between the inlet 216 and the outlet 260. The gases port 220 has a substantially constant diameter as shown. As such, the diameter along the length of the gases port 220 may be substantially the same as the outlet 260 in some embodiments of the connector 200. In this embodiment, the jet flow of gas is delivered by the connector 200 is a result of parameters of the outlet 260, including the hydraulic diameter of the outlet 260 which will be discussed, and/or additional parameters, such as the flow rate of gas received at the inlet 216. The jet flow of gas is delivered at the outlet 260 at a desired target velocity, preferably to achieve one or more of the system objectives described above.

A jet flow of gas is a region of high velocity of gas. The jet flow of gas preferably includes a velocity that is capable of achieving one or more of the system objectives described above. The jet flow of gas preferably includes a velocity that is capable of achieving at least the target patient pressure of at least 2 cmH₂O about the device port 240 when in use. The velocity of the jet flow of gas may be greater or less than the velocity of a gases flow provided or generated by a flow source 110. Preferably, the velocity of the jet flow of gas is greater than the velocity of a gases flow provided or generated by a flow source 110. Additionally or alternatively, the velocity of the jet flow of gas may be greater or less than the velocity of a gases flow elsewhere in the system 100, preferably the velocity of the jet flow of gas is greater than the velocity of a gases flow elsewhere in the system 100. Preferably, the jet flow of gas includes a velocity in a range of about 5 m/s to about 60 m/s, as will be discussed below. Preferably, the jet flow of gas includes a velocity in a range of about m/s to about 60 m/s at a selected flow rate of about 20 L/min to about 70 L/min of the flow of gas provided by the flow source 110. The jet flow of gas may include a velocity in a range of about 5 m/s to about 60 m/s at a selected flow rate of about 20 L/min to about 90 L/min of the flow of gas provided by the flow source 110.

In this embodiment, it is noted that the outlet 260 of the gases port 220 is disposed between the inlet 216 of the gases port 220 and the device port 240. As shown in other embodiments, for example in FIGS. 4 and 6, the outlet 260 may extend into the device port 240, where an adapter 126 is connected to an invasive respiratory device 120. The outlet 260 preferably does not extend into the invasive respiratory device 120 in order to minimise the risk of barotrauma to the subject 300. Thus, it is preferable that the outlet 260 is disposed between the inlet 216 of the gases port 220 and a distal end port 122 of the invasive respiratory device 120 when coupled to the device port 240 (see also FIG. 1).

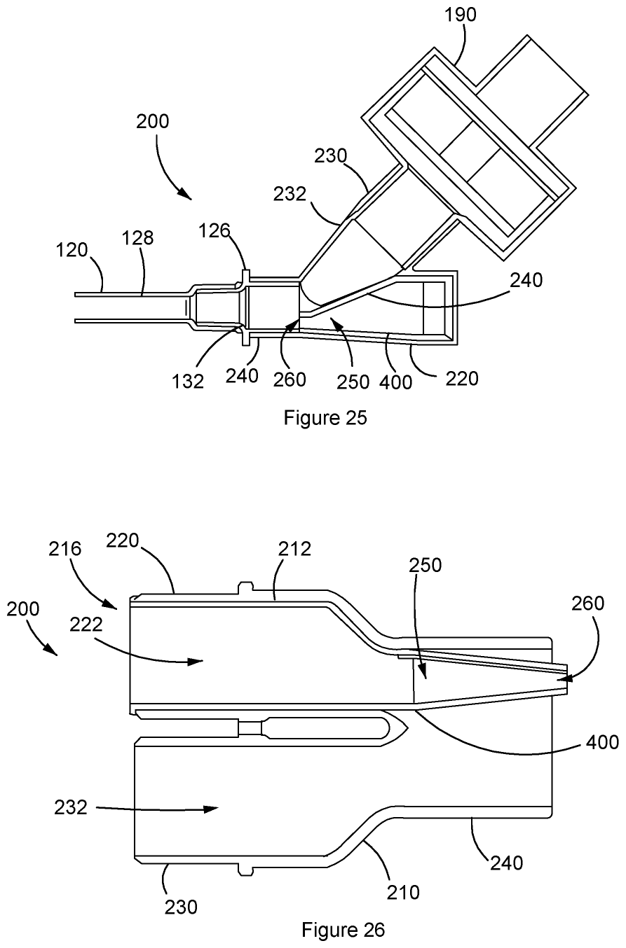

FIGS. 3A and 3B are schematic diagrams of another connector 200 for coupling with an invasive respiratory device 120, according to some embodiments of the invention. In these embodiments, the connector 200 includes an insert 400 which is positionable within an inlet channel 222 of the connector 200 for providing the jet outlet 260 in use (see also FIG. 3B). These embodiments will be described in more detail in connection with connectors 200 having various inserts 400, and also locating features for engagement and locking of the components together, in relation to FIGS. 25 to 30.

FIGS. 4 and 6 are schematic diagrams of further connectors 200 for coupling with an invasive respiratory device 120, according to some embodiments of the invention. Embodiments of the invention as described herein often include an adapter 126 coupling the invasive respiratory device 120 to the device port 240 of the connector 200, as per FIGS. 4 and 6. However, the adapter 126 may be excluded as shown in FIGS. 2 and 3A-B, and the device port 240 may instead be directly coupled to the invasive respiratory device 120, as would be appreciated by a person skilled in the art. As such, embodiments of the invention are not limited to requiring an adapter 126 for connection with the invasive respiratory device 120.

In the connectors 200 of FIGS. 4 and 6, the gases port 220 further includes a flow constriction 250 for providing the jet flow of gas through the outlet 260 of the gases port 220. The flow constriction 250 includes a region of decreasing cross-sectional area, which accelerates the velocity of the gas flow, before exiting through the outlet 260 of the gases port 220 as a jet. The jet flow of gases in these embodiments is therefore the result of a decreasing cross-sectional area which accelerates the gas flow.

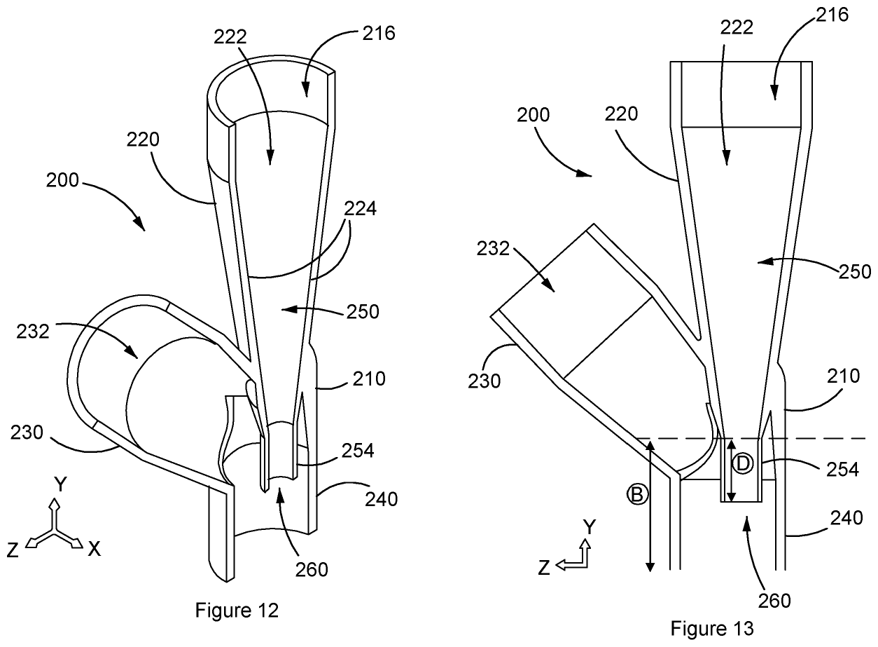

In some embodiments as shown, the flow constriction 250 includes a tapered region or portion for constricting flow of gas prior to exiting the outlet 260. The flow constriction 250 of FIGS. 4 and 6 is in the form of a tapered nozzle having the outlet or opening 260. The tapered region is formed by angled walls 224 of the main body 210 of the connector 200 as illustrated in FIG. 4. A tapered constriction 250 to form a jet flow of the gas is more efficient than a sudden change in cross-sectional area, and the angle of the taper is an important parameter. The angle should be chosen to avoid detachment of the boundary layer when the gas flow leaves the taper and enters either a constant diameter portion 254 (see FIG. 12) or the main body 210 of the connector 200.

An angle of the wall 224 of the tapered region relative to the longitudinal axis 252 of the flow constriction 250 may be in a range of more than 0 degrees to about 45 degrees. Preferably, the angle is between about 2 degrees and about 20 degrees. The angle of the taper may be, for example, between about 2 degrees and about 15 degrees, about 5 degrees and about 20 degrees, about 5 degrees and about 15 degrees, about 10 degrees and about 15 degrees, about 10 degrees and about 20 degrees, and may be about 2, 3, 4, 5, 6, 7, 8, 9, 10, 11, 12, 13, 14, 15, 16, 17, 18, 19 or 20 degrees. The angle of the wall 224 represents the half angles of the tapered region relative to the longitudinal axis 252 which provide an efficient means of constricting the flow.

A jet flow of the gas exits the outlet 260 of the flow constriction 250, which in this embodiment is adjacent to the device port 240 for coupling to the invasive respiratory device 120. Since the gases port 220, flow constriction 250 and device port 240 are coaxial in this embodiment, along axis 252, the jet flow of the gas is delivered centrally into the invasive respiratory device 120 when coupled to the device port 240, such as via the adapter 126 shown in FIG. 4.

During patient expiration, the majority of gases from the jet vent out of the connector 200 through an outlet port 230. That is, the direction of travel of gases is along an expiratory flow path 270 defined between the device port 240 and the outlet port 230 (see also FIGS. 8 and 9). In the embodiment of FIG. 4, the gases from the invasive respiratory device 120 pass through the adapter 126 and around the exterior of the device port 240 coupled to the adapter 126. An outlet port 230 is located between the device port 240 and adapter 126 having a short channel through which gases in the expiratory flow path 270 exit the connector 200 to atmosphere. During inspiration, when the direction of travel of the gas from the jet outlet 260 is towards the invasive respiratory device 120, the jet's momentum generates a desired pressure within the connector 200 and patient 300 which maintains a patent airway.

It is important to note that a jet of gases is provided by the flow constriction 250 of the connector 200 of various embodiments of the invention, and the flow constriction 250 is not located within the invasive respiratory device 120 (or within a device or instrument in the patient's airway). The location of formation of the jet of gases within the connector 200 rather than the invasive respiratory device 120 reduces the probability of barotrauma occurring in the lung through a blockage of the trachea, and is an important advantage of various embodiments of the invention.

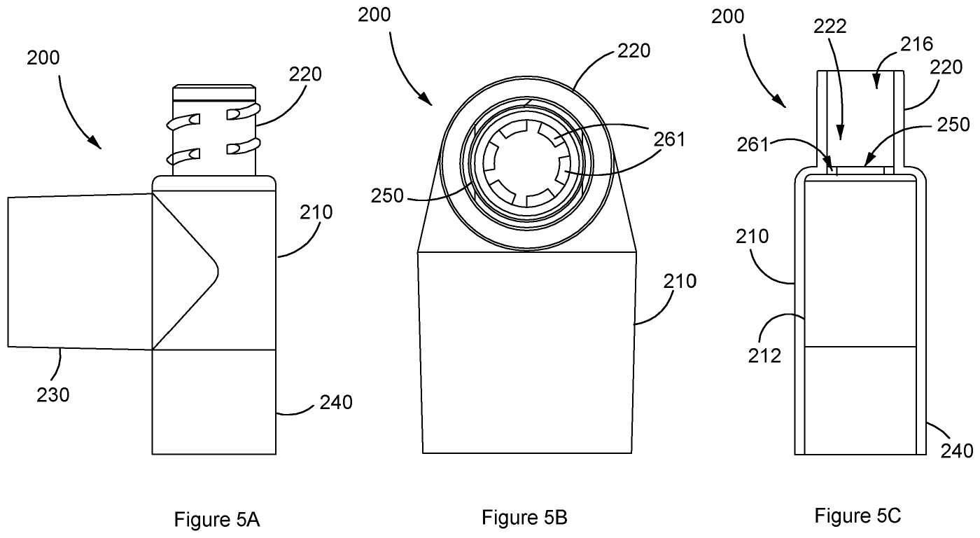

FIGS. 5A-C are schematic diagrams of further connectors 200 for coupling with an invasive respiratory device 120, according to some embodiments of the invention. The flow constriction 250 may be in the form of a closed nozzle having a plurality of openings or apertures 261 as the outlet 260. FIG. 5A illustrates a front view of the connector 200 having similar features to the previous embodiments in terms of a connector main body 210 with a gases port 220, an outlet port 230 and a device port 240. FIG. 5B illustrates a top view of the connector 200 showing a flow constriction 250 formed by a plurality of openings or apertures 261 in the inspiratory flow path 280 (see also FIGS. 6, 10 and 11) through which the flow of gas is jetted into the main body 210 of the connector 200. FIG. 5C illustrates a side cross-sectional view of the connector 200 also showing the flow constriction 250 with plurality of openings or apertures 261. Six openings or apertures 261 are provided in these embodiments in an annular pattern. However, it will be appreciated that embodiments of the invention are not limited to six openings or the particularly spacing as shown. The size, number, shape and/or location of the openings or apertures 261 can be optimised to provide desirable characteristics of the jet flow of the gas for providing respiratory support, especially for providing high flow respiratory support invasively to the patient 300.

In the embodiments of FIGS. 5A-C, the flow constriction 250 does not include a tapered portion. Instead, the increase in velocity to form the jet is due to the decreased size of the jet outlet 260 formed by the plurality of openings or apertures 261, relative to the larger diameters of the gases port 220 and inlet channel 222. Thus, a person skilled in the art would readily appreciate that embodiments of the invention are not limited to requiring nozzles or tapered regions in order to provide the flow constriction 250, nor do they require a flow constriction 250 at all, and that others means are possible to achieve an increased velocity of gas in the connector 200 to form the jet flow.

FIG. 6 shows another connector 200 for coupling with an invasive respiratory device 120 according to some embodiments of the invention. This figure shows an adapter 126 coupled to the device port 240 on the main body 210 of the connector 200, omitting the invasive respiratory device 120 for clarity. The connector 200 includes a gases port 220 and a flow constriction 250 in the form of a tapered nozzle having an outlet 260. The jet flow of gas is delivered into the main body 210 of the connector 200 and travels towards the device port 240 in the inspiratory flow path 280 as indicated by the arrow in solid lines. During expiration, the gas flow direction reverses and flows in the expiratory flow path 270 defined between the device port 240 and the outlet port 230 as indicated by the arrows in broken lines.

In this embodiment, the outlet port 230 is shown as a plurality of openings or apertures 263 for passage of gas flow in the expiratory flow path 270 to the atmosphere. Although the embodiment illustrates six apertures or openings 263, the connector 200 may include any number of apertures or openings for passage of gas flow in the expiratory flow path 270 out of the connector 200, as would be appreciated by a person skilled in the art. Furthermore, the number, size and shape of the apertures may be configured in order to alter the resistance to flow (RTF) of the connector 200.

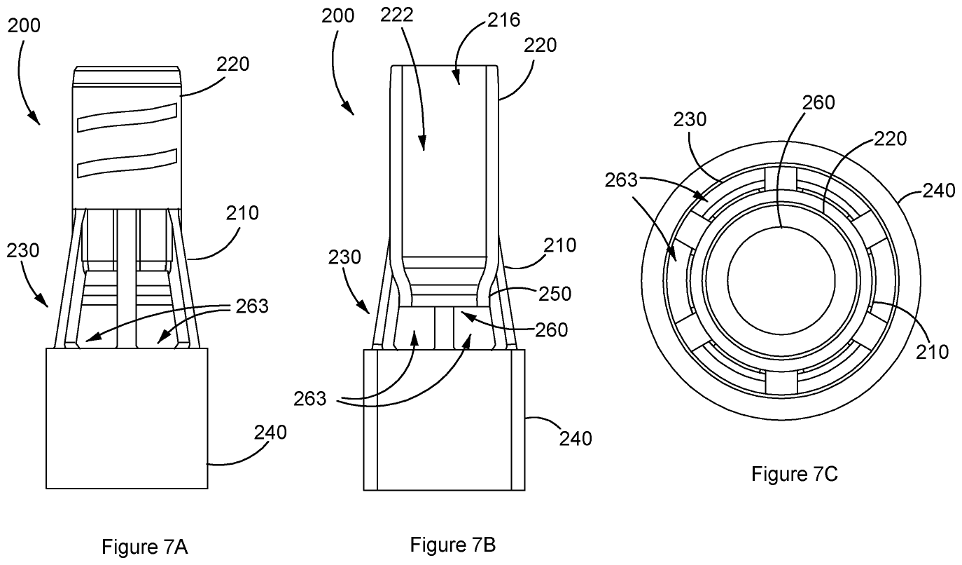

FIGS. 7A-7C illustrate another connector 200, similar to FIG. 6, for coupling with an invasive respiratory device 120 according to some embodiments of the invention. These figures omit the invasive respiratory device 120 for clarity. FIG. 7A illustrates a front view of the connector 200 having similar features to FIG. 6 with the outlet port 230 including a plurality of openings or apertures 263 for passage of gases from the connector 200 to the atmosphere. FIG. 7B illustrates a side cross-sectional view of the connector 200 showing a gases port 220 and a flow constriction 250 in the form of a tapered nozzle having an outlet 260 for jetting the flow of gas into the main body 210 and towards the device port 240. FIG. 7C is a top view of the connector 200 of FIGS. 7A and 7B illustrating more clearly the plurality of openings or apertures 263 of the outlet port 230. As discussed above, the connector 200 may include any number of apertures or openings 263 for passage of gases in the expiratory flow path out of the connector 200 and is not limited to the number, size, shape and/or location of the openings 263 as shown in the embodiments of FIGS. 7A-7C.

### Connector Parameters

FIG. 8 is a schematic diagram of another connector 200 for coupling with an invasive respiratory device 120, having an offset outlet channel 232 and gas sampling ports 214, 234, and indicating parameters of the connector 200, according to some embodiments of the invention.

The connector 200 includes a gases port 220 having an inlet 216 in flow communication with an inlet channel 222, a flow constriction 250 and an outlet 260. In this embodiment, the flow constriction 250 is in the form of a tapered nozzle extending into the main body 210 of the connector 200. However, embodiments of the invention are not limited to a tapered nozzle and may instead include a plurality of openings or apertures 261 as shown in the embodiments of FIGS. 5A-C. The connector 200 also includes an outlet port 230 with an outlet channel 232 that forms part of the expiratory flow path 270 through which gases exit from the connector 200. The expiratory flow path 270 in the connector 200 is defined between the device port 240 and the outlet port 230. A longitudinal axis 236 of the outlet channel 232 is offset relative to a longitudinal axis 228 of the inlet channel 222 (see also FIGS. 17 to 19A-B). The offset is at an obtuse angle relative to the longitudinal axis 228 of the inlet channel 222 such that the outlet port 230 is adjacent the device port 240. In contrast, the connector 200 shown in FIGS. 12 to 19A-B include an offset at an acute angle such that the outlet port 230 is adjacent the gases port 220.

FIG. 9 is a schematic diagram of another connector 200 for coupling with an invasive respiratory device 120, having an offset outlet channel 232 and a flow constriction 250 formed by a tapered nozzle which is substantially aligned with a wall 212 of the main body 210 of the connector. The nozzle is laterally offset relate to a central axis 246 of the device port 240 (see also FIG. 11). It is advantageous for the nozzle to deliver the jet flow of gas, which exits the outlet 260, at or along a wall 212 of the main body 210 of the connector 200. This will be discussed in more detail below.

In relation to FIGS. 8 and 9, there are illustrated three important parameters of the connector 200. Firstly, the outlet 260 of the connector 200 has a diameter (and cross-sectional area) denoted as parameter A. Secondly, there is a desired distance, denoted as parameter B, between the outlet 260 and a proximal end opening 242 of the device port 240. Thirdly, there is a minimum diameter (and cross-sectional area) of the expiratory path 270, denoted as parameter C (notably at different sections of the expiratory path 270 in FIGS. 8 and 9 due to the varying offset of the outlet channel 232 and alignment). The parameters A to C will also be discussed in relation to the embodiments of FIGS. 12 to 19A-B.

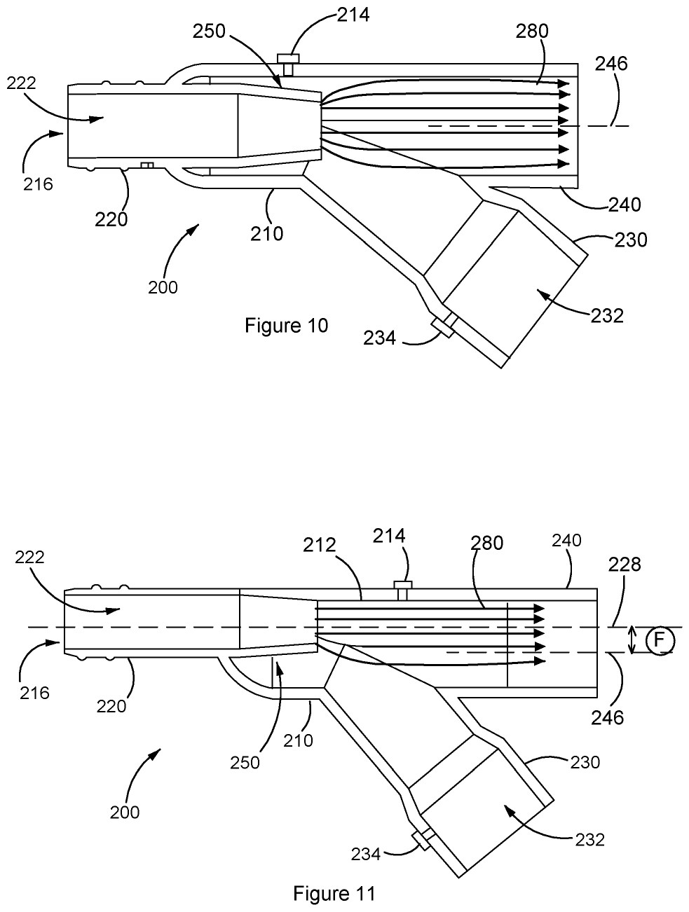

FIGS. 10 and 11 show the inspiratory flow paths 280 of the jet flow of gas exiting the outlets 260 of the connectors 200 of FIGS. 8 and 9, respectively. As can be observed, the jet flow of gas in FIG. 10 exits the outlet 260 and dissipates across the diameter of the main body 210 of the connector 200 between the gases port 220 and the device port 240. In contrast, the jet flow of gas in FIG. 11 remains at least partly attached to a wall 212 of the main body 210 of the connector.

FIG. 11 also shows another parameter of the connector 200, denoted as parameter F. This represents the lateral offset distance between a longitudinal axis 228 of the outlet 260 and a central axis 246 of the device port 240. In this example, the lateral offset is in the direction towards the wall 212 of the main body 210. This enables the jet flow to travel at least partly along the wall 212 of the main body 210, and subsequently, at least partly along a wall 128 of the invasive respiratory device 120 when in use. Additional embodiments showing the lateral offset of the nozzle are described with reference to FIGS. 32A-B to 35A-B. The lateral offset may be defined as a percentage of the distance between the central axis 246 and an inner surface of a wall 212 of the connector 200, where 0% is where the nozzle is aligned with the central axis 246 of the device port 240 and 100% is where the longitudinal axis 228 of the nozzle outlet 260 is closer to the wall 212 of the connector 200 as the percentage tends towards 100%. The lateral offset is preferably less than about 100%, less than about 90%, less than about 80%, less than about 70%, less than about 60%, less than about 50%, less than about 40%, less than about 30%, less than about 20%, less than about 10%, or less than about 5%.

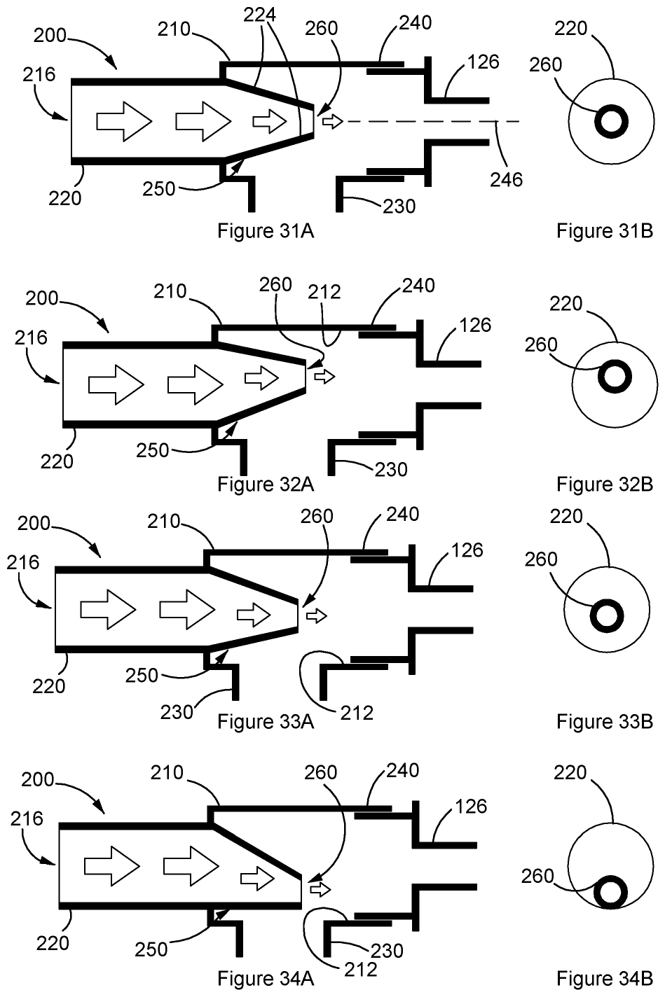

It may be desirable that the tapered nozzle directs inspiratory flow to a wall 212 of the connector 200 before the jet flow proceeds to the invasive respiratory device 120 (e.g., an ETT) coupled in use with the system 100. A jet of gases ‘attaching’ to a wall 212, 244 of the connector 200 and/or a wall 128 of the invasive respiratory device 120 (see also FIGS. 32A-B to 35A-B) may minimise turbulent losses. This is because inspiratory flow that attaches to a wall 128 of the invasive respiratory device 120 provides an expiratory path 270 via the region of the invasive respiratory device 120 that the inspiratory flow is not attached to. This expiratory path 270 may be central if inspiratory flow moves circumferentially around the walls 128 of the invasive respiratory device 120 or it may be on one side of the invasive respiratory device 120 if flow is attaching to the other side on inspiration. For example, the flow can ‘attach’ to the side of the invasive respiratory device 120 closer to the inlet channel 222 and expiratory flow can exit via the side of the invasive respiratory device 120 closer to the outlet channel 232. In alternative embodiments, the inspiratory path 280 may be central and the expiratory flow may move circumferentially around the walls of the invasive respiratory device 120. Various embodiments relating to features of flow attachment and direction in the connector 200 will be described in connection with FIGS. 31A-B to 42 below.

If flow is directed and jetted centrally down the invasive respiratory device 120, inspiratory/expiratory flow can coincide causing turbulent/pressure losses and increasing RTF (see also FIG. 31A-B). If there is substantial coinciding and/or colliding and/or interfering and/or overlap of the inspiratory and expiratory flow paths 280, 270, the opposing flow directions interact and cause turbulent losses, increasing the RTF of the connector 200. The increased RTF increases the required driving pressure and the expiratory resistance (i.e., expiratory RTF) of the connector 200. An increased expiratory RTF increases the work of breathing of a spontaneously breathing patient 300.

Flow attachment occurs when flow of gas attaches to a wall(s) of the connector 200 and/or the invasive respiratory device 120. Flow attachment can be beneficial across all patients 300 (spontaneously and non-spontaneously breathing) as generating a desired patient pressure is important for oxygenation and airway CO₂ clearance. Furthermore, flow attachment may be especially beneficial to spontaneously breathing patients where a reduced expiratory resistance (i.e., reduced expiratory RTF of the connector 200) reduces the work of breathing of a patient 300.

FIGS. 8 to 11 also show that the connector 200 may further include one or more gas sampling ports 214, 234 for sampling one or more characteristics of the gases in the main body 210 of the connector 200. The one or more characteristics of the gases sampled at the ports 214, 234 may include pressure, flow rate, concentration, gas constituents (e.g., oxygen and/or carbon dioxide), temperature, humidity, contaminants, aerosols and/or pathogens. The connector 200 may include one or more gas sampling ports 234 located on the outlet port 230, such as for sampling levels of contaminants, aerosols and/or pathogens located in the gases exiting the main body 210. FIGS. 8 to 11 show a single gas sampling port 234 on the outlet port 230, although embodiments may include a plurality of ports, and the one or more ports may be located at any location along a length of the outlet port 230 having the outlet channel 232.

In some embodiments, the connector 200 may include one or more gas sampling ports 214 located on the main body 210, such as for sampling pressure levels of the gases in the main body 210. As shown in FIGS. 8 to 11, the gas sampling port 214 may be located near the jet outlet 260. Although not shown, the gas sampling port 214 may be located along a length of the device port 240. FIGS. 8 to 11 show a single gas sampling port 214, although embodiments may include a plurality of ports as would be appreciated by a person skilled in the art, for sampling pressure levels of gases at various points in the connector 200.

In some embodiments, the connector 200 may also include a suctioning port (not shown). The suctioning port may be located in the same or similar positions on the connector main body 210 or outlet port 230 of the connector 200 as the gas sampling ports 214, 234. The suctioning port may provide for removal of bodily fluids from the connector 200, such as mucus.

Various parameters of inventive connectors 200 according to embodiments of the invention will now be described in connection with FIGS. 12 to 19A-B. Certain parameters will be identified that have been found to contribute to the inventive aspects relating to systems 100 for providing respiratory support to a subject 300.

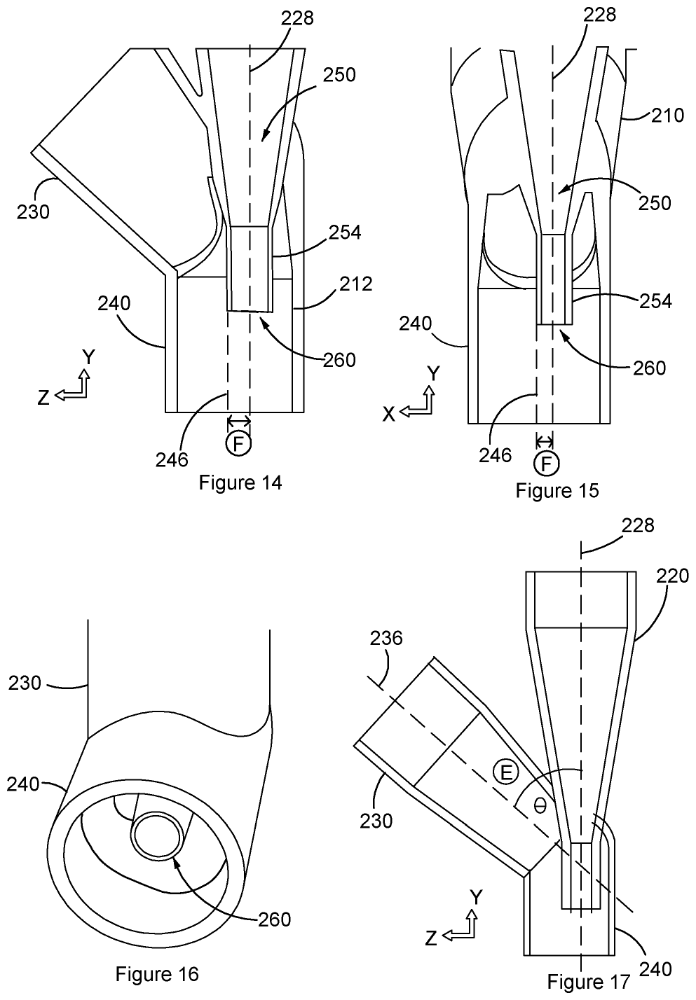

FIG. 12 is a perspective cross-sectional view of another connector 200 for coupling with an invasive respiratory device 200, showing an offset outlet channel 232 and a nozzle having a constant diameter portion 254, according to some embodiments of the invention. FIGS. 13 and 14 are cross-sectional views of the connector of FIG. 12 in the Z-Y plane, indicating parameters of the connector 200.

In this embodiment, the offset of the outlet channel 232 relative to the inlet channel 222 is at an acute angle such that the outlet port 230 and the gases port 220 are adjacent. This is more clearly shown in FIG. 17, which is a simplified cross-sectional view of the connector 200 of FIG. 12 in the Z-Y plane. The acute angle E is provided between a longitudinal axis 228 of the inlet channel 222 and a longitudinal axis 236 of the outlet channel 232. The acute angle E of the offset outlet channel 232 may be, for example, in the range of about 5 degrees to about 90 degrees, about 10 degrees to about 80 degrees, about 20 degrees to about 70 degrees, about 30 degrees to about 60 degrees, about 40 degrees to about 50 degrees, and may be about 5, 10, 15, 20, 25, 30, 35, 40, 45, 50, 55, 60, 65, 70, 75, 80, 85 or 90 degrees.

The flow constriction 250 is provided in the form of a tapered nozzle with walls 224 of the tapered region as illustrated in FIG. 12. Prior to the jet exiting the outlet 260 of the nozzle, the gases port 220 includes a conditioning portion 254. The conditioning portion 254 may be adjacent the outlet 260 and may have a substantially constant cross-sectional area for conditioning the flow of gas prior to exiting the outlet 260. The conditioning portion 254 may be located between the tapered region having the tapered walls 224 of the flow constriction 250 and the outlet 260, as shown in FIG. 12. As shown in FIG. 13, the conditioning portion 254 has a length, denoted as parameter D. Preferably this length D is in a range of more than 0 mm to about 60 mm.

FIG. 13 also shows the corresponding parameter B from FIG. 8 for embodiments where the conditioning portion 254 is not present. However, in embodiments that include the conditioning portion 254, the desired distance from the outlet 260 of the jet nozzle to a distal end portion 122 of the invasive respiratory device 120 would be the parameter length B minus the additional length D of the conditioning portion 254.

Advantageously, the conditioning portion 254, which may include a substantially constant diameter and/or cross-sectional area, may condition the flow such that it is directed in a desired direction towards or along a wall 212 of the main body 210 of the connector or towards or along a wall 128 of the invasive respiratory device 120 when in use. Furthermore, a constant diameter portion 254 may reduce pressure dissipation out of the outlet 260 when compared with a jet outlet 260 that immediately follows the tapered region of the flow constriction 250.

The advantages of the conditioning portion 254 may be appreciated with respect to the embodiments illustrated in FIGS. 14, 15, 18A-B and 19A-B, which are cross-sectional views of the connector 200 of FIG. 12 through various planes.

FIG. 14 illustrates a cross-sectional view of the connector 200 of FIG. 12 in the Z-Y plane. As can be observed, the conditioning portion 254 enables direction of the jet flow of gases along a longitudinal axis 228 of the inlet channel 222. Thus, the jet flow of gases exiting the outlet 260 will not dissipate significantly and will remain as a more targeted jet flow along the longitudinal axis 228, and into the invasive respiratory device 120 (not shown). The nozzle of FIG. 14 is also illustrated as being laterally offset by a distance F and aligned towards a wall 212 of the main body 210 of the connector 200. Accordingly, there may be at least some attachment of the boundary layer of the jet flow, as previously discussed with respect to the connector embodiments of FIGS. 9 and 11, thereby minimising turbulence losses in the connector 200.

Similarly, FIG. 15 shows another view of the connector 200 of FIG. 12 in the X-Y plane with a laterally offset nozzle. As shown, the diameter of the outlet 260 and conditioning portion 254 is smaller than that shown in the view of FIG. 14, which will result in an elliptical cross-section for the outlet 260. The slightly elliptical shape of the outlet 260 is indicated in the enlarged view of the connector 200 as shown in FIG. 16 viewed from the device port 240. An elliptically shaped outlet 260 or generally a non-circular outlet 260, can be oriented to increase the cross-sectional area (CSA) of the expiratory flow path 270, when compared to a circular-shaped outlet 260. Furthermore, an elliptically shaped outlet 260 enables a larger cross-sectional area of the outlet 260 which is advantageous when other parameters of the connector 200 are fixed. However, other embodiments of the invention may include a circular cross-section for the outlet 260, as will be discussed with reference to FIG. 39A-B.

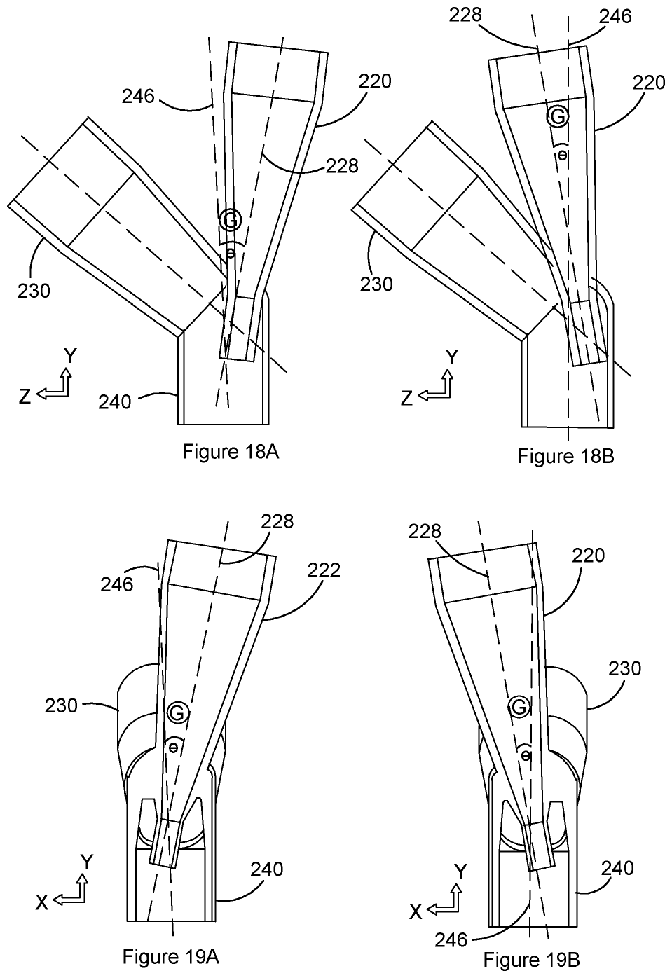

FIGS. 18A and 18B show simplified views of the corresponding connector view as illustrated in FIG. 14. However, in these embodiments the nozzle is angled relative to a central axis 246 of the device port 210. The offset angle, denoted as parameter G, is defined between the central axis 246 of the device port 210 and the longitudinal axis 228 of the inlet channel 222. In FIG. 18A, the inlet channel 228 is offset at an angle G in a direction towards the outlet port 230. FIG. 18B illustrates the offset angle G being in a direction away from the outlet port 230.

FIGS. 19A and 19B show simplified views of further embodiments of the connector 200 in the X-Y plane (corresponding to the view as illustrated in FIG. 15). In these embodiments, the nozzle is angled relative to a central axis 246 of the device port 210, directed along or towards a wall of the invasive respiratory device 120 when in use (towards left side in FIG. 19A and towards right side in FIG. 19B).

The inventors have determined parameters of the connector 200 that influence performance with respect in particular to the system objectives described herein. There are some trade-offs when optimising the connector parameters to meet certain objectives as would be understood by a person skilled in the art. In particular, there is a desired minimum patient pressure and a range of flows that can be provided which meets this minimum pressure (where the flow meets the patient inspired flow in a spontaneous breathing situation). Losses in generated pressure depend on the rate of energy dissipation in the fluid leaving the nozzle which depends on some of these parameters.

Connector with Jet Flow Velocity

In another inventive aspect, there is provided a connector 200 for coupling with an invasive respiratory device 120. The connector 200 includes a main body 210 having a gases port 220 for receiving a flow of gas from a flow source 110 at a selected flow rate, an outlet port 230 for outflow of gases from the main body 210, and a device port 240 couplable with the invasive respiratory device 120. The gases port 220 includes an inlet 216 and an outlet 260. The connector 200 is configured to receive the flow of gas from the flow source 110 via the inlet 216 of the gases port 220, and to deliver a jet flow of gas through the outlet 260 of the gases port 220. The jet flow of gas delivered through the outlet 260 of the gases port 220 has a velocity in a range of about 5 m/s to about 60 m/s.

Preferably, the jet flow of gas includes a velocity in a range of about 5 m/s to about 60 m/s at a selected flow rate of about 20 L/min to about 70 L/min of the flow of gas provided by the flow source 110. The jet flow of gas may include a velocity in a range of about 5 m/s to about 60 m/s at a selected flow rate of about 20 L/min to about 90 L/min of the flow of gas provided by the flow source 110.

The diameter or cross-sectional area A of the jet outlet 260 is one of the main factors that controls RTF and flow penetration down the invasive respiratory device 120 (e.g., an ETT). The smaller the jet diameter, the higher the jet velocity for a given flow and therefore, the higher the static pressure generated at the ETT entrance for a given flow. However, frictional and turbulent losses will increase as the jet diameter decreases. Thus, higher pressures are required to drive the same flow through the system 100 for smaller jet diameters and a higher driving pressure is necessary to generate the same pressure in the ETT.

The inventors have found that by optimising the parameter representing the outlet diameter and/or cross-sectional area A of the flow constriction 250, they are able to achieve a velocity of about 5 m/s to about 60 m/s of the jet flow exiting the outlet 260. The velocity may be, for example, in a range of about 5 m/s to about 50 m/s, about 10 m/s to about 50 m/s, about 10 m/s to about 40 m/s, about 20 m/s to about 40 m/s, about 30 m/s to about 50 m/s, about 30 m/s to about 40 m/s, and may be about 5, 10, 15, 20, 25, 30, 35, 40, 45, 50, 55, or 60 m/s.

This velocity may be achieved by the outlet 260 having a hydraulic diameter in a range of about 2 mm to about 10 mm. Preferably, the hydraulic diameter is in a range of about 5 mm to about 8 mm, preferably for embodiments where the outlet 260 has a circular cross-section. The hydraulic diameter of the outlet 260 may be, for example, in the range of about 3 mm to about 9 mm, about 4 mm to about 8 mm, about 5 mm to about 7 mm, and may be about 2, 3, 4, 5, 6, 7, 8, 9 or 10 mm, or about 2.5, 3.5, 4.5, 5.5, 6.5, 7.5, 8.5 or 9.5 mm.

Furthermore, the outlet 260 may have a cross-sectional area (CSA) in a range of about 10 mm² to about 60 mm². Preferably, the cross-sectional area is in a range of about 19 mm² to about 50 mm² (based on the circular cross-section with diameter 5-8 mm²). The CSA of the outlet 260 may be, for example, in a range of about 10 mm² to about 50 mm², about 20 mm² to about 50 mm², about 25 mm² to about 45 mm², about 30 mm² to about 40 mm², about 35 mm² to about 45 mm², and may be about 10, 15, 19, 20, 25, 30, 35, 40, 45 or 50 mm₂.

The hydraulic diameter, D_{H}, of the outlet 260 may be defined as follows:

$${D}_{H} = \frac{4 A}{P}$$

where
A is the cross-sectional area of the outlet 260 of the connector 200,
P is the wetted perimeter of the cross-section of the outlet 260 of the connector 200, and
the bulk vector direction of the flow is normal to the cross-sectional area A.

The distance B of the jet nozzle outlet 260 from the invasive respiratory device 120 is another important parameter. This distance B is a determinant of the generated patient pressure. In some embodiments, a distance B from the outlet 260 to a distal end portion 122 of the invasive respiratory device 120 when coupled to the device port 240 is in a range of about 0 mm to about 60 mm. Preferably, the distance B is in a range of about 10 mm to about 30 mm.

An outlet 260 which is too close to the invasive respiratory device 120 can increase the expiratory resistance to flow (RTF) of the connector 200. If the patient 300 is spontaneously breathing, this will increase the work of breathing and the lung pressure excursion required to maintain a given per minute volume (flow). Thus, there is a balance required and optimal jet diameter range and distance from invasive respiratory device 120. This balance can be shown in terms of a ratio between jet area to distance from the ETT. In some embodiments, a ratio of the cross-sectional area of the outlet 260 of the gases port 220 to the distance from the outlet 260 of the gases port 220 to the distal end portion 122 of the invasive respiratory device 120 is between about 1:1 and about 1:10. More preferably, the ratio is between about 1:1 and about 1:5. The ratio may be, for example, between about 1:1 and about 1:9, between about 1:2 and about 1:8, between about 1:3 and about 1:7, between about 1:4 and about 1:6, or may be about 1:1, 1:2, 1:3, 1:4, 1:5, 1:6, 1:7, 1:8, 1:9 or 1:10.

In some embodiments, the adapter 126 of the invasive respiratory device 120 and/or the connector 200 may include a stop or locating feature to maintain a desired distance B between the outlet 260 and a distal end portion 122 of the invasive respiratory device 120 when coupled to the device port 240 (see FIGS. 3A and 3B). At least one locating feature may be positioned on the device port 240 and/or the main body 210 of the connector 200, such as on an internal or external surface of the device port 240 and/or main body 210. The at least one locating feature may prevent over-insertion or insufficient insertion of the connector 200 into an invasive respiratory device 120. This will help to maintain and ensure an ideal distance B or range of distances of the jet outlet 260 from the invasive respiratory device 120.

The at least one locating feature may include an engagement structure for releasably coupling the device port 240 or main body 210 of the connector 200 with the invasive respiratory device 120 or an adapter 126 connected to the invasive respiratory device 120. The engagement structure may include one or more of a protrusion, a rib, a groove and a flange on an internal or external surface or wall of the device port 240 or main body 210.

Returning to the embodiments of FIGS. 3A and 3B, there is illustrated a connector 200 which includes a nozzle insert 400, which is able to be coupled to the connector 200 by locating features of the connector 200 and/or insert 400. The insert 400 with the nozzle and outlet 260 will be described in more detail in connection with the embodiments of FIGS. 25 to 30. The connector 200 may include one or more protrusions 248 in the main body 210 of the connector 200 as illustrated in FIGS. 3A and 3B. The protrusion 248 may be on a wall of the main body 210 as shown. The protrusion 248 may act as a locating feature to maintain a desired distance B from the outlet 260 of the insert 400 when positioned in the connector 200. When the insert 400 is positioned in the connector 200 as shown in FIG. 3B, a notch 450 on the insert 400 may engage with the protrusion 248 on the main body 210 of the connector 200. The engagement may provide releasable or non-releasable coupling of the connector 200 and insert 400. Although a protrusion 248 and a notch 450 is illustrated, a person skilled in the art would appreciate that the locating features and engagement structure could have any form that provides for releasably or non-releasable coupling of the components together.

Connector with Minimum Expiratory Path Area

In another inventive aspect, there is provided a connector 200 for coupling with an invasive respiratory device 120. The connector 200 includes a main body 210 having a gases port 220 for receiving a flow of gas from a flow source 110 at a selected flow rate, an outlet port 230 for outflow of gases from the main body 210, and a device port 240 couplable with the invasive respiratory device 120. The gases port 220 includes an inlet 216 and an outlet 260. The connector 200 is configured to receive the flow of gas from the flow source 110 via the inlet 216 of the gases port 220, and to deliver a jet flow of gas through the outlet 260 of the gases port 220. The connector 200 further includes an expiratory flow path 270 defined between the device port 240 and the outlet port 230. The expiratory flow path 270 has a minimum cross-sectional area of at least about 25 mm².

The inventors have found that a minimum opening or cross-sectional area C of the expiratory flow path 270, as illustrated in FIGS. 8 and 9, in the connector 200 is an important parameter for embodiments of the invention. The minimum cross-sectional area (CSA) of the expiratory flow path 270 is at least about 25 mm². The minimum CSA of the expiratory flow path 270 may be at least about 30 mm². The minimum CSA of the expiratory flow path 270 may be at least about 35 mm².

In the embodiments described herein, such as with reference to FIGS. 8 to 19A-B, the inspiratory and expiratory flows are separate, and flow enters and exits through the corresponding separate ports. The inspiratory flow enters at the inlet 216 of the gases port 220 and typically travels through an inlet channel 222 in fluid communication with the inlet 216 of the gases port 220. The gases port 220 which may optionally include the flow constriction 250 such as in the form of a tapered nozzle. The expiratory flow enters at the device port 240 and travels from the device port 240 through an outlet channel 232 in fluid communication with the outlet port 230, which the expiratory flow exits to atmosphere (optionally via a filter 190).

The jet of gases exiting the outlet 260 in the inspiratory flow path 280 aims to generate a desired patient pressure to maintain airway patency. The CSA of the expiratory flow path 270, defined between the device port 240 and the outlet port 230, can be tuned to achieve desired results in terms of patient pressures, RTF and driving pressure. An exemplary method of tuning the connector 200 for desired results in terms patient pressures, RTF and/or driving pressure includes providing a gases flow at a set flow rate through the connector 200 and altering the CSA of the expiratory flow path 270 until the desired results are achieved. This method can be used to tune the other features (e.g. shape and/or size of the outlet 260, distance of the outlet 260 to the distal end portion 122 of the invasive respiratory device 120, etc.) of the connector 200 as disclosed the specification herein. A minimum CSA for the expiratory flow path 270 aims to limit the pressure that the patient 300 experiences during expiration and work required for exhalation. Accordingly, it is desirable that a minimum CSA of the expiratory flow path 270 is greater than a CSA of the jet outlet 260. The CSA of the expiratory flow path 270 being greater than the CSA of the jet outlet 260 may desirably provide a lower expiratory RTF and resistance to expiration for the subject 300.

In some embodiments, a ratio of the minimum cross-sectional area C of the expiratory flow path 270 to the CSA of the outlet 260 of the gases port 220 may be between about 2:1 and about 3:1. In some embodiments, the ratio may be between about 2.5:1 and about 3:1, between about 2:1 and about 2.5:1, and the ratio may be about 2:1, 2.5:1 or 3:1. The minimum CSA of the expiratory flow path 270 may be located at any point along the path 270, such as along the outlet channel 232, at the outlet port 230, or preferably, at the entrance of the expiratory flow to the outlet channel 232, which is shown adjacent the outlet 260 as illustrated in FIG. 9 and indicated by parameter C. Preferably, a cross-sectional area of the outlet channel 232 of the outlet port 230 is greater than a cross-sectional area of the outlet 260, as shown in many embodiments of the connector 200.

As illustrated with reference to FIGS. 8 and 9, it is desirable that the flow constriction 250 of the connector 200 is located between the inlet 216 of the gases port 220 and the device port 240 such that it does not obstruct the expiratory flow path 270. Thus, the jet flow of the gas exiting the outlet 260 travels in the inspiratory flow path 280 and does not occlude the expiratory flow path 270. In various embodiments of the connector 200, the flow constriction 250 may be disposed in the inspiratory flow path 280, defined between the inlet 216 of the gases port 220 and the device port 240, and more preferably, is associated with the inlet channel 222, such as including a tapered nozzle in the inlet channel 222 as illustrated in the embodiments of FIGS. 8 to 19A-B. Other embodiments will now be described which show exemplary connectors 200 and show the flow constriction 250 formed in different arrangements.

Connector with Integrally Formed Flow Constriction

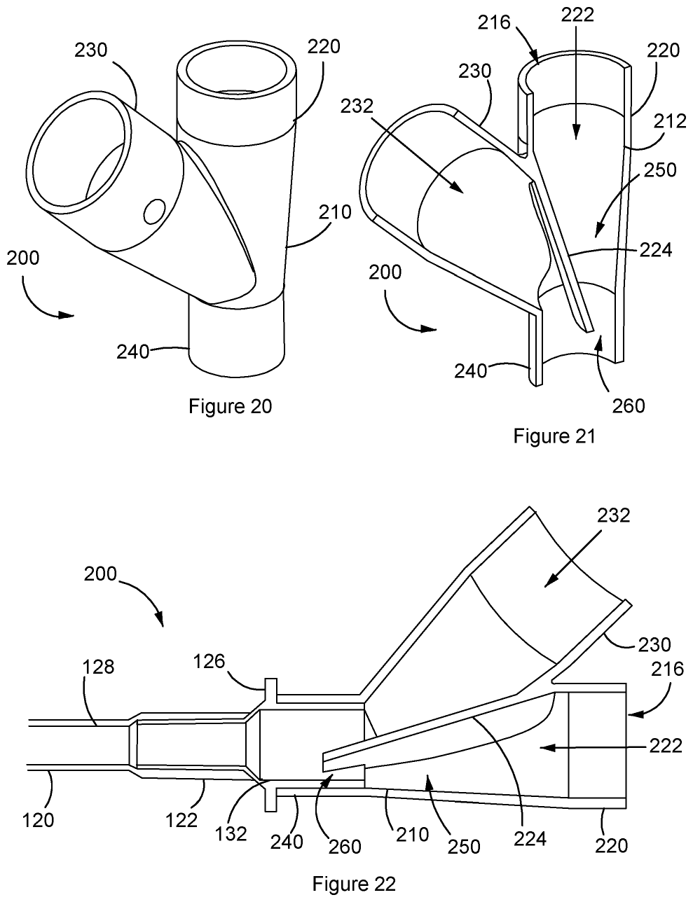

FIGS. 20 to 24 show embodiments of a connector 200 for coupling with an invasive respiratory device 120 that includes an integrally formed flow constriction 250. The flow constriction 250 elements can be integrally moulded during formation of the connector 200. However, embodiments of the invention are not limited to having integrally formed flow constrictions 250 or nozzles with jet outlets 260, as would be appreciated by a person skilled in the art. The connector 200 also includes an offset outlet channel 232 adjacent the inlet channel 222, similar to the offset outlet channel 232 described with reference to the connectors 200 of FIGS. 12 to 19A-B.

As shown in FIG. 21, which is a perspective cross-sectional view of the connector 200 of FIG. 20, the integrally formed flow constriction 250 is located in the inlet channel 222 of the connector 200. The flow constriction 250 is formed between a tapered wall 224 of the inlet channel 222 and a wall 212 of the main body 220 of the connector. The decreasing cross-sectional area of the tapered portion formed between the walls 224 and 212 includes an opening 260 through which a jet flow of gas exits into the main body 210 of the connector 200. The tapered wall 224 is angled such that the outlet or opening 260 directs the jet flow towards or along the wall 212 of the main body 210 of the connector 200. The taper as shown in FIGS. 21 and 22 is asymmetric. In particular, the half angle of the wall 224 of the taper relative to a longitudinal axis 252 of the flow constriction 250 may be in a range of more than 0 degrees to about 45 degrees, and is preferably between about 2 degrees and about 20 degrees, as described with respect to the embodiment of FIG. 4. As previously described, this enables flow attachment of the jet to the wall 212 of the main body 210, and also aids in keeping and/or maintaining the expiratory and inspiratory flow paths 270, 280 separate from one another, thereby enabling delivery of desired patient pressures from the jet outlet 260 while minimising the expiratory flow pressure to the patient 300.

FIG. 22 is a cross-sectional view of the connector 200 of FIG. 20 shown coupled to an adapter 126 connected to an invasive respiratory device 120, according to some embodiments of the invention. The tapered portion of the flow constriction 250 is such that the jet outlet or opening 260 is defined by the internal tapered wall 224 and part of the internal wall 132 of the adapter 126 coupled to the distal port 240. Thus, the jet flow is directed along or towards a wall 132 of the adapter 126, and consequently, along or towards a wall 128 of the invasive respiratory device 120 connected thereto. This also minimises the distance B from the jet flow outlet 260 to a distal end portion 122 of the invasive respiratory device 120 connected to the adapter 126. Advantageously, this reduces dissipation of the jet flow, enabling desired patient pressures to be readily delivered to the subject 300, with lower RTF and driving pressures of the system 100 and connector 200.

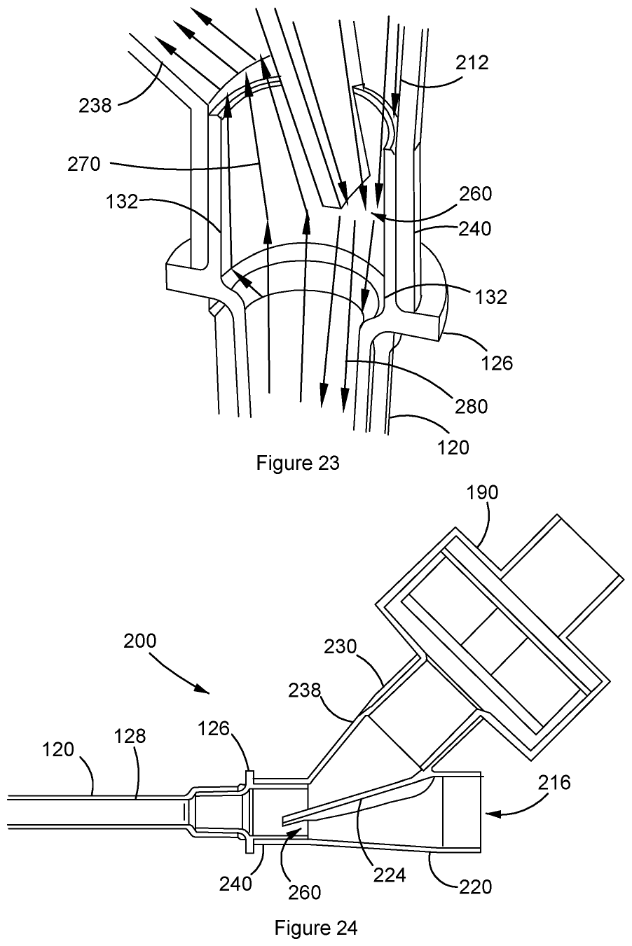

FIG. 23 illustrates an enlarged view of the coupling part of FIG. 22 showing the inspiratory flow path 280 and expiratory flow path 270 through the connector 200 and adapter 126. As can be observed, the inspiratory flow path 280 travels towards and along a wall 212 of the main body 210, and exits the opening 260 as a jet flow of gas directed towards and along a wall 132 of the adapter 126. Consequently, the jet flow of gas then travels towards and along a wall 128 of the invasive respiratory device 120 (see also FIGS. 4, 22, 24, 25 and 27). The expiratory flow path 270 travels towards and along a wall 132 of the adapter 126 on the opposing side of the connector 200, travels along wall 238 of the outlet channel 232 and exits the outlet port 230 to atmosphere (not shown for simplicity).

FIG. 24 is a cross-sectional view of the connector of FIG. 20 coupled to an adapter 126 connected to an invasive respiratory device 120, and showing a filter 190 coupled to the outlet port 230. The filter 190 may be releasably coupled to the outlet port 230 and may be inserted into the outlet port 230 by engagement with a wall 238 of the outlet channel 230, such as by frictional engagement. In other embodiments, the filter 190 may be non-removable and/or integrally formed with the outlet port 230 (see embodiments of FIGS. 53 to 65 with filter 190). The filter 190 may be a radial filter.

Connector with Nozzle Insert

In another inventive aspect, there is provided a connector 200 for coupling with an invasive respiratory device 120. The connector 200 includes a main body 210 having a gases port 220 having an inlet 216 for receiving a flow of gas from a flow source 110 at a selected flow rate, an outlet port 230 for outflow of gases from the main body 210, and a device port 240 couplable with the invasive respiratory device 120. The connector 200 further includes an inlet channel 222 in flow communication with the inlet 216 of the gases port 220. The connector 200 is configured to receive an insert 400 positionable within the inlet channel 222 for providing an outlet 260. The connector 200 is configured to receive the flow of gas from the flow source 110 via the inlet 216 of the gases port 220, and to deliver a jet flow of gas through the outlet 260 provided by the insert 400.

FIGS. 3A, 3B and 25 to 30 show alternative embodiments of the connector 200, in which the flow constriction 250 with outlet 260 provided by an insert 400 which is positioned within the inspiratory flow path 280, and preferably within the inlet channel 222 as shown. The insert 400 of FIGS. 3A, 3B and 25 to 30 may be removable or non-removable upon insertion within the inlet channel 222. A benefit of insert 400 is that the characteristics of the flow constriction 250 may be more readily and accurately customised than if the flow constriction nozzle and/or insert 400 was formed integrally with the connector 200. However, an integrally formed flow constriction 250, such as by having a tapered wall 224 forming a nozzle and/or an integral insert 400, is beneficial for ease of manufacturing and for the user/operator as there are fewer components to assemble for the system 100.

As referred to previously, FIGS. 3A and 3B provide an insert 400 couplable with a connector 200, according to some embodiments of the invention. The insert 400 includes a flow constriction 250 in the form of a nozzle having the outlet 260. When the insert 400 is coupled to the connector 200, as per the protrusion 248 and notch 450 as previously described and shown in FIG. 3B, the connector 200 is configured to deliver a jet flow of gas through the outlet 260 provided by the insert 400.

FIG. 25 is a cross-sectional view of another connector 200 in a similar configuration as FIG. 24 with an adapter 126 and filter 190 coupled thereto. The connector 200 however includes an insert 400 located in the inlet channel 222, according to another arrangement. The insert 400 includes a tapered wall 420 which forms the flow constriction 250 and an opening 260 forming the jet outlet. The half-angle of the tapered wall 420 is preferably in a range of more than 0 degrees to about degrees, and preferably between about 2 degrees and about 20 degrees, similar to the tapered walls 224 of the nozzle described in relation to previous embodiments. Furthermore, the opening 260 of the insert 400 is located such that the jet flow is directed along or towards a wall 132 of the adapter 126 and into the invasive respiratory device 120.

FIG. 26 is another embodiment of the connector 200 in the form of a wye-piece connector, showing another embodiment of the insert 400 in the inlet channel 222 to form a flow constriction 250 when in use. The insert 400 can be removably coupled with the gases port 220 of the connector 200. Alternatively, the insert 400 may be non-removable and/or formed integrally with the connector 200. The insert 400 includes a tapered nozzle and an outlet 260 as shown, which extends beyond the device port 240. However, the nozzle is ideally sized and located such that it does not extend into the invasive respiratory device 200 when coupled thereto, such as by use of an adapter 126 (not shown). In this embodiment, the inlet channel 222 and outlet channel 232 are located adjacent to one another.

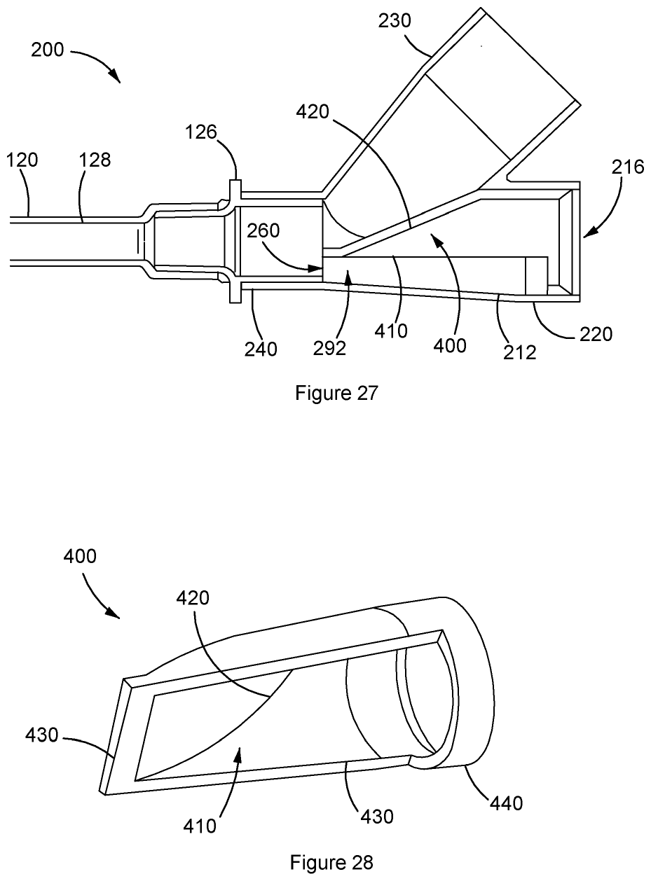

FIG. 27 illustrates another connector 200 according to some embodiments, having a similar configuration to FIG. 20 with an adapter 126 coupled to the device port 240 with an invasive respiratory device 120, such as an ETT, connected thereto. However, the insert 400 of the embodiment of FIG. 27 and related FIGS. 28 to 30 is modified from the insert 400 shown in FIG. 25.

In particular, the insert 400 of this embodiment is best shown in FIG. 28. The insert 400 has an opening 410 formed by a region of reduced wall thickness of the insert 400. The reduced wall thickness and opening 410 extends substantially along a length of the insert 400, although this could be adjusted depending on desired flow characteristics in the connector 200. The insert 400 also includes a region of reduced cross-sectional area formed by a tapered wall 420. When the insert 400 is positioned in the inlet channel 222 as shown in FIG. 27, the opening 410 of the insert 400 results in formation of a fluid gap or passage 292 between the insert 400 and a wall 212 of the main body 210 of the connector 200. Due to the insert 400 having a tapered wall 420, similar to that described with respect to the embodiment of FIG. 25, a flow constriction is formed by the fluid gap or passage 2929 and a jet flow of gas exits through an opening or outlet 260 formed between the insert 400 and a wall 212 of the main body 210 of the connector 200.

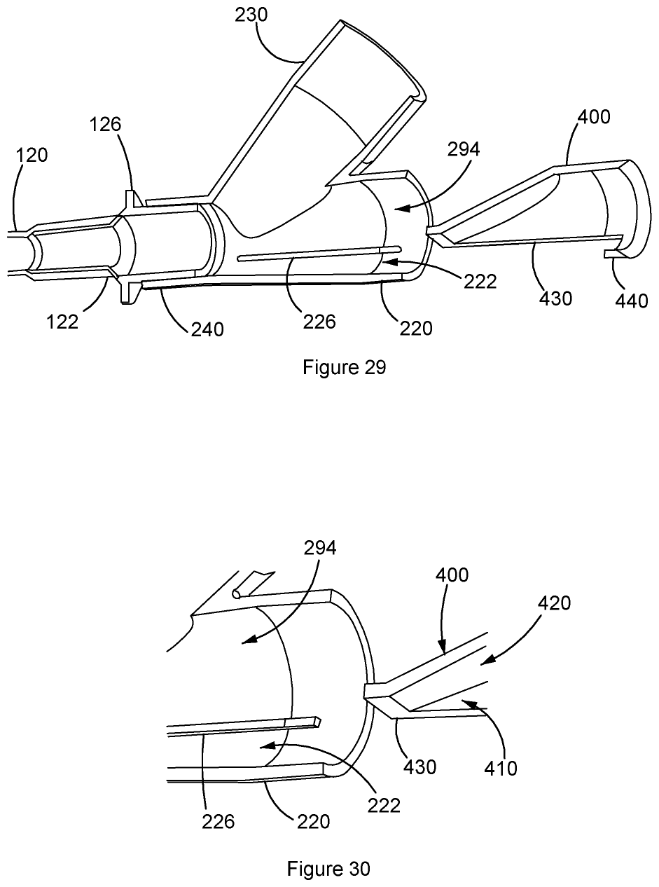

FIGS. 29 and 30 are schematic views showing guiding of the insert 400 of FIGS. 27 and 28 into the connector 200. The insert 400 is positioned in the inlet channel 222 by engaging with at least one locating feature of the connector 200, which is illustrated as a rib 226 on a wall 294 of the inlet channel 222. The inlet channel 222 may comprise other locating features on the internal wall 294 to locate and hold the insert 400 in position in the connector 200. The locating features may include one or more of a protrusion, a groove or a flange on a wall 294 of the inlet channel, to name a few. In other embodiments, the at least one locating feature of the connector 200 may include locating features on an external wall of the connector 200, such as on an external wall of the inlet port 220 or body 210 of the connector 200, or on a wall of the insert 400. Various possible locating features would be appreciated by a person skilled in the art, which would enable the location and fixation of the insert 400 in the connector 200.

The locating feature of the connector 200 can also assist with the insert 400 and the jet nozzle 260 being maintained at a known desired distance B from a distal end portion 122 of the invasive respiratory device 120. As previously described, the distance B of the jet outlet 260 from the distal end portion 122 of the invasive respiratory device 120 is an important parameter and should be maintained in a desired range. A user or operator may select or be able to manually adjust the distance B by selecting or adjusting a desired length of the insert 400 located within the inlet channel 222. Alternatively, separate inserts 400 with different lengths may be provided to achieve different distances from the distal end portion 122 of the invasive respiratory device 120.

The insert 400 also includes at least one locating feature to guide positioning within the inlet channel 222. The locating feature may include the region of reduced cross-sectional area having the opening 410 of the insert 400 as shown in FIGS. 29 and 30 for engaging with a wall 294 of the inlet channel 222. The insert 400 provides guiding surfaces 430 adjacent the opening 410 for slidably engaging with the rib 226 in the inlet channel 222. Additionally, the insert 400 includes a stop portion 440 for engaging with an edge of the rib 226 as best shown in FIG. 27, preventing further insertion of the insert 400 into the connector 200. However, other locating features may be included in the insert 400, such as the notch 450 of the insert 400 of FIGS. 3A and 3B, as would be readily appreciated by a person skilled in the art, and that the insert 400 is not limited to having the guiding surfaces 430, a stop 440 or a notch 450, as illustrated.

### Nozzle Insert

In another inventive aspect, there is provided an insert 400 for a connector 200 couplable with an invasive respiratory device 120. The connector 200 includes a main body 210 having a gases port 220 including an inlet 216 for receiving a flow of gas from a flow source 110 at a selected flow rate, an outlet port 230 for outflow of gases from the main body 210, and a device port 240 couplable with the invasive respiratory device 120. The connector 200 further includes an inlet channel 222 in fluid communication with the inlet 216 of the gases port 220. The insert 400 is configured to be positioned in the inlet channel 222 of the connector 200 to provide an outlet 260. The connector is configured to receive the flow of gas from the flow source 110 via the inlet 216 of the gases port 220, and to deliver a jet flow of gas through the outlet 260 provided by the insert 400.

The insert 400 may include one or more of the features as described in connection embodiments of the invention of the connector 200 having insert 400 shown in FIGS. 3A, 3B and 25 to 30.

Connector with Nozzle Outlet Offset

In previous embodiments described, the outlet 260 of the connector 200 has often been coaxial with the device port 240 and invasive respiratory device 120 when coupled thereto. This is illustrated in FIGS. 31A-B, which show an embodiment of another connector 200 having a jet nozzle which is substantially aligned with a central axis 246 of the device port 240, and the outlet 260 is coaxial with the invasive respiratory device 120. However, in other embodiments described, the outlet 260 has been substantially aligned or directed towards a wall 212 of the main body 210 of the connector 200 and/or a wall 128 of the invasive respiratory device 120 when coupled to the device port 240. This alternative aspect of embodiments of the invention will now be described.

In the embodiments shown in FIGS. 31A-B to 41 and 43 to 48 and 50A-B, the invasive respiratory device 120 has been omitted for clarity and instead an adapter 126 is shown coupled to the device port 240. The adapter 126 may be connected to an invasive respiratory device 120 when in use. A person skilled in the art would appreciate that the invasive respiratory device 120 may be connected to the adapter 126 such that it is coaxial with the device port 240 and adapter 126 as shown in these embodiments. It should also be understood that the adapter 126 may be excluded and the invasive respiratory device 120 may be coupled directly to the device port 240. Furthermore, reference to a wall 128 of the invasive respiratory device 120 in these embodiments can be understood with reference to FIGS. 4, 22, 24, 25 and 27.

In another inventive aspect, there is provided a connector 200 for coupling with an invasive respiratory device 120. The connector 200 includes a main body 210 having a gases port 220 for receiving a flow of gas from a flow source 110 at a selected flow rate, an outlet port 230 for outflow of gases from the main body 210, and a device port 240 couplable with the invasive respiratory device 200. The gases port 220 includes an inlet 216 and an outlet 260. The connector 200 is configured to receive the flow of gas from the flow source 110 via the inlet 216 of the gases port 220, and to deliver a jet flow of gas through the outlet 260 of the gases port 220. The outlet 260 of the gases port 220 is offset relative to a central axis 246 of the device port 240 for directing the jet flow of gas along or towards a wall 212 of the main body 210 of the connector 200 and/or a wall 128 of the invasive respiratory device 120 when coupled to the device port 240 (see FIGS. 32A-B to 35A-B).

In some embodiments, the outlet 260 may be substantially aligned with or directed towards a wall 212 of the main body of the connector 200 and/or a wall 128 of the invasive respiratory device 120 coupled to the device port 240. These embodiments will be described in relation to FIGS. 32A-B to 35A-B. FIGS. 32A-B to 35A-B show that the direction of flow towards the walls 212, 128 may be achieved by providing a laterally offset outlet 260 relative to the central axis 246 of the device port 240, which relates to connector parameter F pertaining to the lateral offset distance as previously described. This is provided by an angle and orientation of the tapered walls 224 of the nozzle forming the flow constriction 250 (see also FIG. 31A). Flow which is directed at or along a wall 212, 128 has the benefits as described earlier including minimising turbulent losses by providing a central expiratory flow area and path in the invasive respiratory device 120.

In FIGS. 32A-B to 37A-B, the lateral offset of the nozzle outlets 260 is due to the angle of the tapered wall(s) 224 as previously described. FIG. 31A-B shows that the nozzle has a conical shaped taper in contrast to FIGS. 32A-B to 37A-B which have a non-conical shaped taper. The angle of the taper is altered in FIGS. 32A-B to 37A-B in order to change the direction of the flow, such as particularly towards or along a wall 212 of the main body 210 of the connector and/or towards or along a wall 128 of the invasive respiratory device 120. As such, modifying the angle of the taper can affect the flow characteristics in the connector 200 and invasive respiratory device 120.

FIGS. 32A-B and 33A-B illustrate another embodiment of a connector 200 having a jet nozzle outlet 260 directing flow towards a wall 128 of the invasive respiratory device 120 (not shown, see e.g., FIGS. 4, 22, 23, 25 and 27) when in use, showing a schematic view (FIGS. 32A and 33A) and an end view (FIGS. 32B and 33B). FIG. 32B shows that the outlet 260 is oriented in relation to the gases port 220 on a side away from the outlet port 230. In contrast, the outlet 260 of FIG. 33B is oriented in relation to the gases port 220 on a side towards the outlet port 230. This orientations of the outlets 260 of FIGS. 32B and 33B would result in jet flow being directed towards a wall 128 of the invasive respiratory device 120 when in use. As can be observed, the jet nozzle outlet 260 is laterally offset by a distance (previously denoted as parameter F) relative to the central axis 246 of the device port 240 in order to achieve the jet flow direction in this embodiment.

FIGS. 34A-B and 35A-B illustrate another embodiment of a connector 200 having a jet nozzle outlet 260 directing flow substantially along a wall 212 of the main body 210 of the connector 200, showing a schematic view (FIGS. 34A and 35A) and an end view (FIGS. 34B and 35B). FIGS. 34A-B show orientation of the jet outlet 260 towards a wall 212 of the main body 210 on a side near the outlet port 230. In contrast, the orientation is towards a wall 212 of the main body 210 on a side away from the outlet port 230 in FIGS. 35A-B. FIGS. 34B and 35B illustrate the orientation of the jet outlet 260 in relation to the gases port 220, which would provide jet flow being directed substantially along a wall 212 of the main body 210 of the connector 200. As can be observed, the jet nozzle outlet 260 is laterally offset relative to the central axis 246 of the device port 240 in order to achieve the jet flow direction in this embodiment.

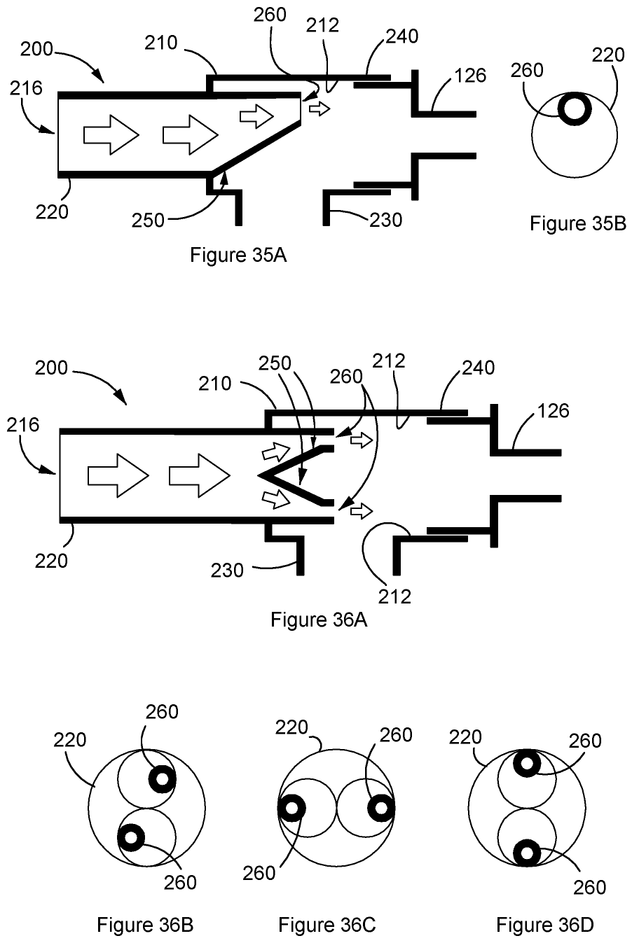

As shown in FIGS. 36A-D, the connector 200 may include two or more nozzles forming the flow constriction 250 and have two outlets 260 that are laterally offset relative to a central axis 246 of the device port 240, in some embodiments of the invention. FIGS. 36A-D illustrate arrangements of the two outlets 260 directing flow towards or substantially along a wall 128 of the invasive respiratory device 120 (not shown, see e.g., wall 128 in FIGS. 4, 22, 23, 25 and 27). The connector 200 may include a flow constriction 250 formed by two tapered nozzles having the two outlets 260 as shown in the schematic view of FIG. 36A. FIG. 36D shows an end view of the two outlets 260 of FIG. 36A. FIGS. 36B and 36C show end views of the two outlets 260 with alternative orientations in relation to the gases port 220 for directing flow towards or substantially along a wall 128 of the invasive respiratory device 120.

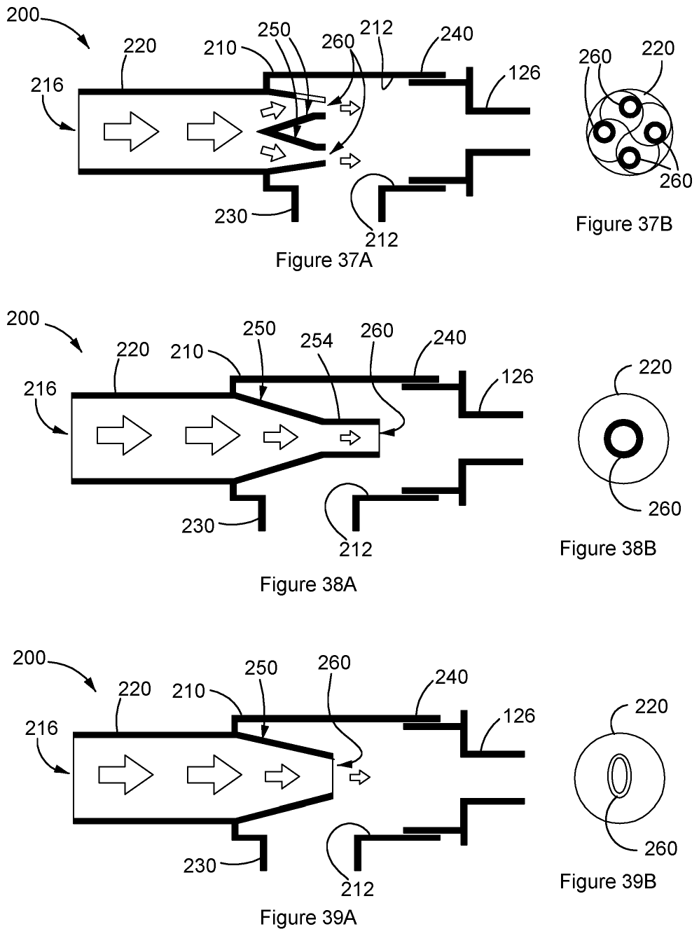

FIGS. 37A-B show another embodiment in which the connector 200 includes four nozzles forming the flow constriction 250 and having four outlets 260 that are laterally offset relative to a central axis 246 of the device port 240. FIGS. 37A-B illustrate an arrangement of four outlets 260 directing flow towards a wall 212 of the main body 210 of the connector 200. The connector 200 may include a flow constriction 250 formed by four tapered nozzles having the four outlets 260 as shown in the schematic view of FIG. 37A (two outlets omitted for simplicity). FIG. 37B is an end view showing orientation of the four outlets 260 in relation to the gases port 220.

FIGS. 38A-B illustrate another connector 200 including a nozzle with a conditioning portion 254 adjacent the jet outlet 260. The conditioning portion 254 includes a substantially constant cross-sectional area or diameter as shown in the schematic view of FIG. 38A. The conditioning portion 254 may be aligned with a central axis of the invasive respiratory device 120 when in use. The end view of FIG. 38B shows the central alignment of the outlet 260 in relation to the gases port 220. The conditioning portion 254 has similar features to the conditioning portion 254 illustrated in FIGS. 12 to 19A-B. Advantageously, the constant diameter portion 254 may condition the flow such that it is directed in a desired direction in the connector 200. Furthermore, the constant diameter portion 254 may reduce pressure dissipation out of the outlet 260 when compared with a jet nozzle outlet 260 that immediately follows the tapered region of the nozzle forming the flow constriction 250. The conditioning portion 254 and invasive respiratory device 120 are coaxial as shown in the end view of FIG. 38B, such that the flow is directed in a central axis of the invasive respiratory device 120.

FIGS. 39A-B illustrate another embodiment of a connector 200 in which the flow constriction outlet 260 has an elliptical cross-section as shown in the schematic of FIG. 39A, although the elliptical shape is particularly evident in the end view of FIG. 39B. It should be noted that the jet nozzle outlet(s) 260 of the connectors 200 of any of the embodiments described herein may have any cross-sectional shape such as square, rectangle, triangular, oval, etc., to name a few. The majority of the embodiments illustrated in the figures show an outlet 260 having a circular-shaped cross-section. A circular outlet 260 is shown in many embodiments as this shape provides a more uniform flow distribution. Thus, shapes other than circular will increase the RTF of connector 200 comparatively. Nonetheless, it will be appreciated that embodiments of the invention are not limited to have a circular cross-section for the outlet 260.

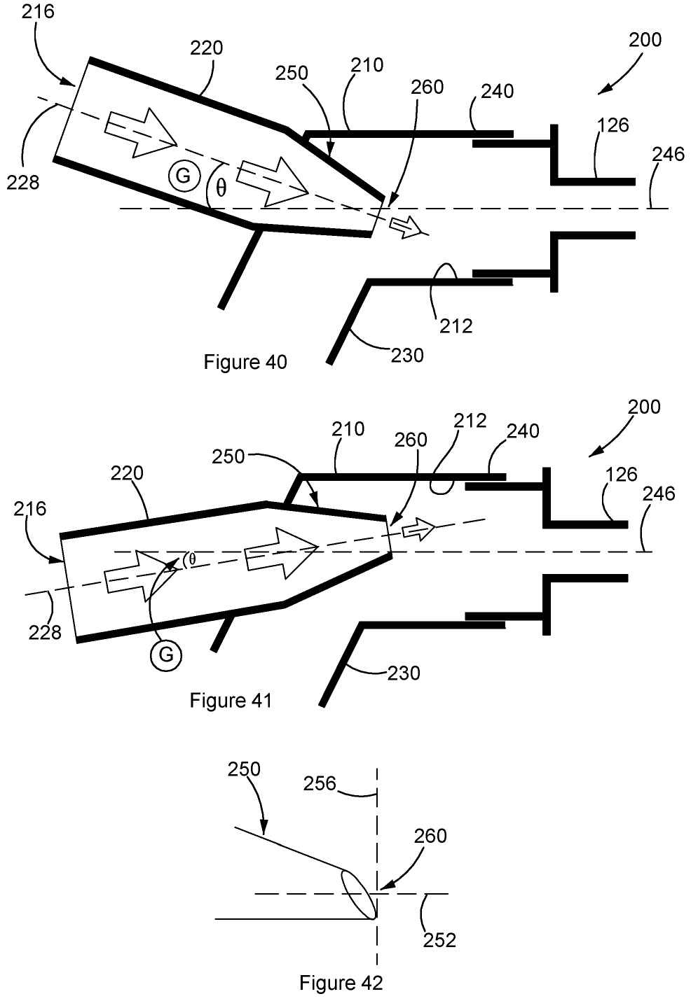

The previous connector embodiments show the longitudinal axis 228 through the gases port 220 as being aligned with the direction of flow through the invasive respiratory device 120. However, in some embodiments, the outlet 260 is angularly offset relative to the central axis 246 of the device port 240. This is shown in the embodiments of FIGS. 40 and 41 which include an offset angle, denoted as connector parameter G, of the longitudinal axis 228 relative to the central axis 246 of the device port 240. The offset angle G may be oriented towards the outlet portion 230 (FIG. 40) or away from the outlet port 230 (FIG. 41). The offset angle G may be in a range of more than 0 degrees to about 180 degrees. Preferably, the offset angle G is in a range of more than 0 degrees to about 135 degrees. More preferably, the offset angle G is in a range of more than 0 degrees to about 90 degrees.

FIGS. 40 and 41 illustrate exemplary connectors 200 having a tapered nozzle angled at an offset angle G towards a wall of the main body 210 of the connector 200, in the direction towards the outlet port 230 (FIG. 40), and away from the outlet port (FIG. 41). This has the effect of angling the direction of flow through the device port 240 and subsequently, the invasive respiratory device 120. These angled nozzles further help to direct the jet of flow towards a wall 212 of the main body 210 of the connector 200 or towards a wall 128 of the invasive respiratory device 120.

In some embodiments, the outlet 260 may include an angled opening relative to a transverse axis 256 of the flow constriction 250 for directing the jet flow of the gas along or towards a wall 212 of the main body 210 of the connector 200 and/or a wall 128 of the invasive respiratory device 120 when coupled to the device port 240. Effectively, the jet nozzle outlet 260 may be ‘cut off’ at an angle relative to the transverse axis 256 of the flow constriction 250 to direct flow out of the outlet 260 in a desired direction, e.g., along or towards walls of the main body 210 or invasive respiratory device 120. An exemplary embodiment of a nozzle with an angled or “cut off” outlet 260 is shown in FIG. 42. The angled opening of the nozzle in FIG. 42 may be about 45 degrees. The angled opening 260 may be in a range of more than 0 degrees to about 90 degrees relative to the transverse axis 256 of the flow constriction 250. This feature can be used separately or in combination with the angled nozzles shown in FIGS. 40 and 41, or in any of the other embodiments of the connectors 200 as described herein.

Connector with Fluidic Switch Mechanism

According to another inventive aspect, there is provided a connector 200 for coupling with an invasive respiratory device 120. The connector 200 includes a main body 210 having a gases port 220 for receiving a flow of gas from a flow source 110 at a selected flow rate, an outlet port 230 for outflow of gases from the main body 210, and a device port 240 couplable with the invasive respiratory device 200. The gases port 220 includes an inlet 216 and an outlet 260. The connector 200 is configured to receive the flow of gas from the flow source 110 via the inlet 216 of the gases port 220, and to deliver a jet flow of gas through the outlet 260 of the gases port 220. The connector 200 is configured to change the direction of gas flow within the main body 210 of the connector 200 when in use.

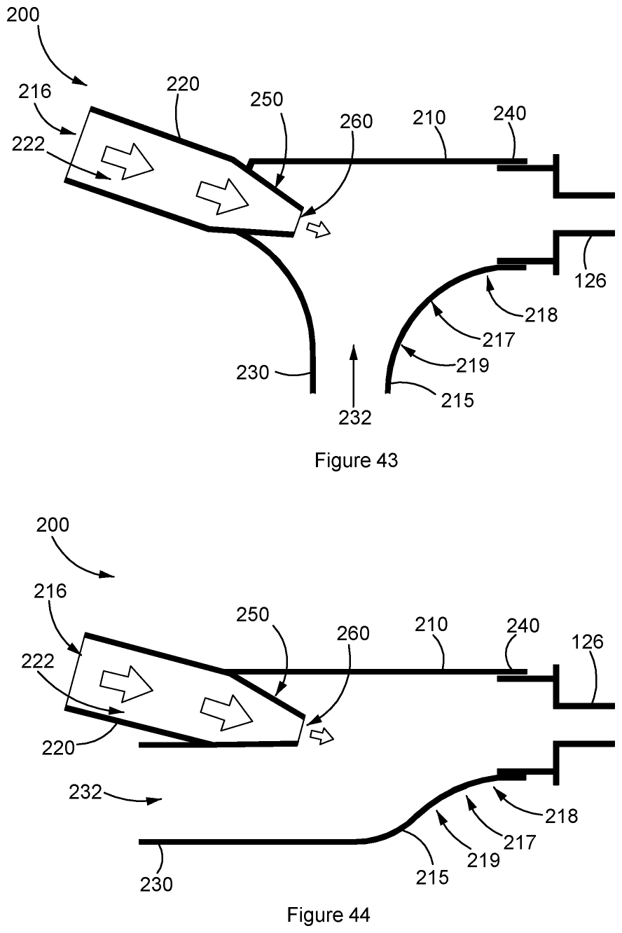

FIGS. 43 to 47 are schematic views of connectors 200 for coupling with an invasive respiratory device 120, having a fluidic flip or switching mechanism, according to some embodiments of the invention. The connectors 200 are able to change the direction of gas flow within the main body 210 of the connector 200 through the fluidic flip or switching mechanism. This mechanism may enable the connector 200 to passively change the direction of gas flow in response to inspiration and/or expiration of the subject 300, as will be described.

More specifically, the connectors 200 of FIGS. 43 to 47 include a tapered angled nozzle, similar to the embodiment of FIG. 40, directed towards a wall 215 of the main body 210 of the connector 200 opposing the outlet 260. Effectively, the gases port 220 and the inspiratory flow path 280 are angled and directed towards an elbow 217 of an opposing wall 215 of the main body 210 of the connector 200. In these embodiments, the inspiratory flow is directed centrally out of the nozzle outlet 260 at the opposing wall 215 and angled slightly towards the device port 240. Preferably, the opposing wall 215 is curved or sloped as shown in FIGS. 43 to 45. In other embodiments, only part of the opposing wall 215 may be curved as shown in FIG. 46. In other embodiments still, the opposing wall 215 may include two straight portions joined at an elbow 217 as shown in FIG. 47.

In the embodiments shown in FIGS. 43 to 47, the connector 200 is configured to direct the jet flow of the gas towards the device port 240 during inspiration of the subject 300 and towards the outlet port 230 during expiration of the subject 300. This is achieved due to the relative arrangement of the angled nozzle and opposing wall 215 of the main body 210 which enables this fluidic flip or switching mechanism. During inspiration, the jet flow of the gas attaches or flows along the side 218 of opposing wall 215 in the direction of the device port 240. During expiration, the direction of the jet flow of the gas changes and becomes angled slightly towards the expiratory flow path 270 and the outlet port 230. This helps to draw the expiratory flow through the outlet channel 232, potentially making it easier for the patient 300 to breathe.

In relation to FIGS. 43 to 45 which include a curved or sloped opposing wall 215, the Coanda effect may result in attachment of the jet to the wall 215. During inspiration, jetting flow may attach to a side 218 of the elbow 217 of the wall 215 closest to the device port 240 and then proceeds towards the invasive respiratory device 120. When there is expiratory flow, the jet detaches from the opposing wall 215, and the direction swaps and becomes angled slightly towards the expiratory flow path 270 and the outlet 230. The jet then attaches to the other side 219 of the elbow 217 of the curved wall 215 closer to the outlet channel 232 and helps to draw the expiratory flow through the outlet channel 232.

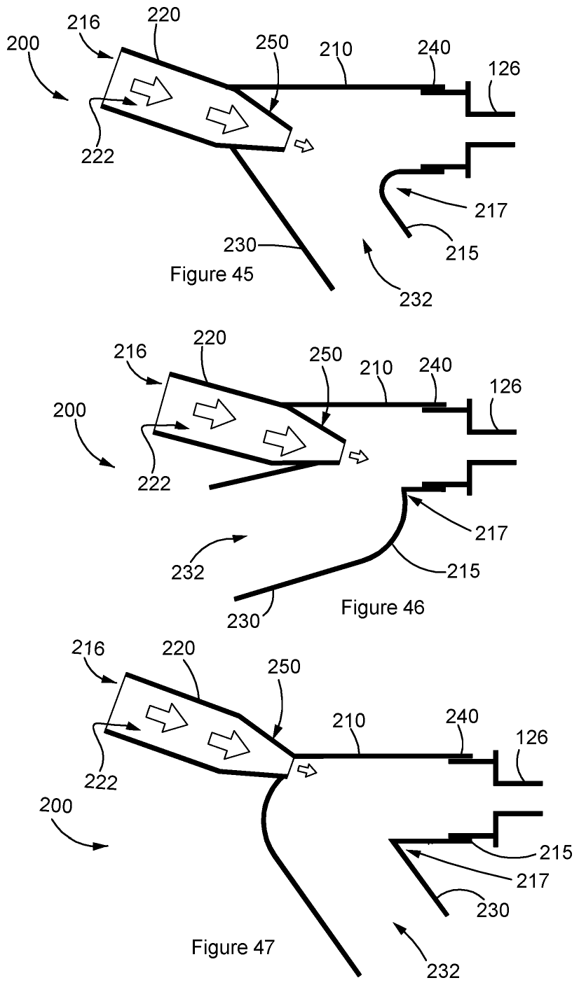

In these embodiments, the opposing wall 215 forms at least part of a wall 238 of the outlet port 230. However, embodiments of the invention are not limited to this arrangement and the opposing wall 215 may be located on a section of the main body 210 of the connector 200 at a distance from the outlet port 230. Additionally, in some embodiments, such as illustrated in FIGS. 45 and 47, the device port 240 and the outlet port 230 may be located at an acute angle relative to each other. However, again this is not limiting in view of the other embodiments illustrated in FIGS. 44, 45 and 46.

Connector with Flow Altering Feature

According to another inventive aspect, there is provided a connector 200 for coupling with an invasive respiratory device 120. The connector 200 includes a main body 210 having a gases port 220 for receiving a flow of gas from a flow source 110 at a selected flow rate, an outlet port 230 for outflow of gases from the main body 210, and a device port 240 couplable with the invasive respiratory device 200. The gases port 220 includes an inlet 216 and an outlet 260. The connector 200 is configured to receive the flow of gas from the flow source 110 via the inlet 216 of the gases port 220, and to deliver a jet flow of gas through the outlet 260 of the gases port 220. The connector 200 further includes at least one flow altering feature for altering at least one characteristic of the jet flow of gas exiting the outlet 260.

The connector 200 may have a flow altering feature associated with the flow constriction 250, the outlet 260, the inlet 216 of the gases port 220 and/or the inlet channel 222. The flow altering feature may be configured to create or increase a degree of turbulent or chaotic flow of the jet flow of gas exiting the outlet 260, as will be described. The characteristics altered may include, for example, one or more of direction, velocity, divergence, spread, profile and/or turbulence (e.g., Reynolds number) of the jet flow exiting the outlet 260. For a selected flow rate, the presence of a flow altering structure may alter or change these characteristics of the flow jetting out of the outlet 260 to achieve desirable characteristics based on the intended respiratory support being provided.

For example, the presence of a flow altering structure may alter flow characteristics such that desirable respiratory support can be achieved with a larger jet outlet diameter A, potentially reducing the necessary driving pressure in the system 100 whilst maintaining the RTF of the connector 200 and the patient pressure. Additionally or alternatively, these structures may create a spiralled or chaotic flow in the invasive respiratory device 120, which can have benefits as discussed below.

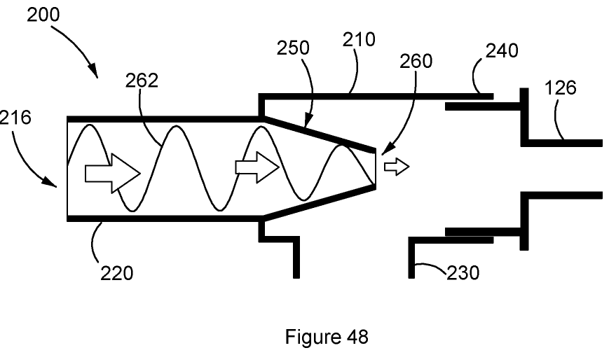

FIG. 48 is a schematic view showing another connector 200 with a jet nozzle having a spiral structure 262 for producing spiral flow, according to some embodiments of the invention. The spiral or screw-like structure 262 can be moulded or placed within the nozzle. The structure 262 may cause flow to spiral in the nozzle and continue to spiral after exiting the outlet 260, creating a cyclonic effect in the invasive respiratory device 120 when in use. This spiralling flow may attach to an inner wall 128 of the invasive respiratory device 120 when being delivered to the patient 300. This inspiratory flow spiralling around the inner wall 128 of the invasive respiratory device 120 provides an easy, low resistance expiratory flow path out the centre of the invasive respiratory device 120 to the outlet channel 232. The spiralling flow may also decrease pressure loss due to turbulence when the spiralling flow is attached to the inner wall 128, and reduces the driving pressure requirements of the system 100.

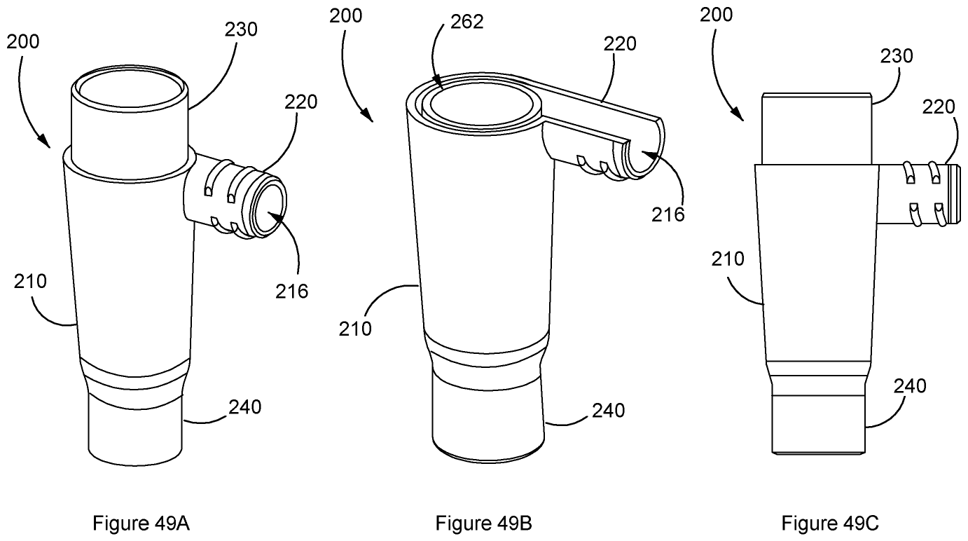

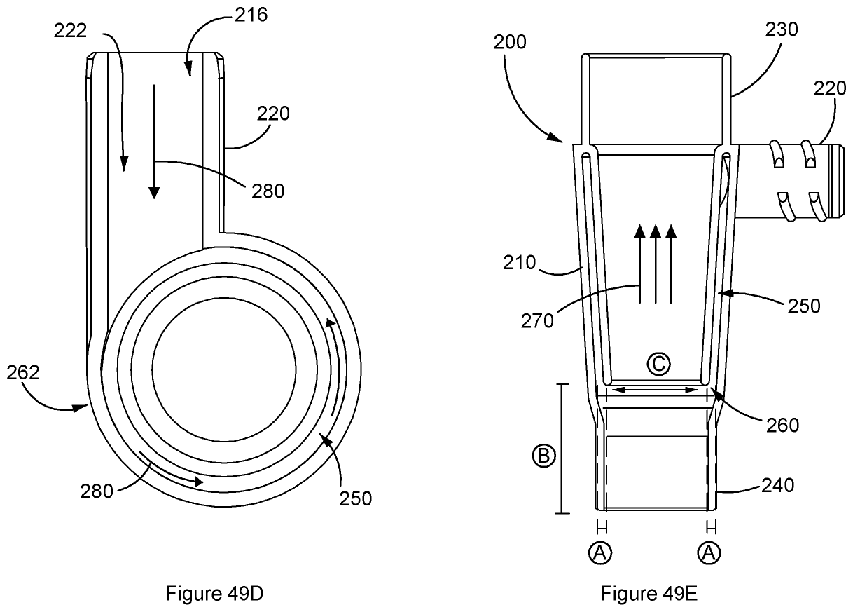

FIGS. 49A-E are schematic views showing another connector 200 with a jet nozzle having a spiral structure 262 for producing spiral flow, according to some embodiments of the invention. FIG. 49A illustrates a perspective view of the connector 200 including a main body 210 with a gases port 220, outlet port 230 and device port 240. FIG. 49B illustrates a sectional view through the gases port 220 of FIG. 49A showing the spiral structure 262 and FIG. 49C illustrates a side view of the connector 200. FIG. 49D shows a top cross-sectional view of the spiral structure 262 through the gases port 220 and FIG. 49E shows a side cross-sectional view of the connector 200 illustrating the jet outlet 260. Notably, FIG. 49E illustrates important parameters A, B and C of various embodiments of the connector 200 (which correspond to the same parameters as shown in FIGS. 8 and 9). Parameter A represents the minimum cross-sectional area of the jet outlet 260, parameter B represents the distance between the outlet 260 and a proximal end opening 242 of the device port 240 and parameter C represents the minimum cross-sectional area of the expiratory flow path 270.

Referring to FIG. 49D, flow enters the gases port 220 tangentially via the inlet 216 and into the inlet channel 222 and is directed to the spiral structure 262 which increases the speed/velocity of the gases as the inspiratory flow path 280 (see arrows) narrows towards the jet outlet 260. The minimum cross-sectional area of the jet outlet 260 is indicated by the parameter A in FIG. 49E and controls the velocity of the jet flow of gas being delivered in this embodiment. The gas flows centrally in the expiratory flow path 270 (see arrows) and exits the outlet port 230. Spiral flow may beneficially deliver flow further through the invasive respiratory device 210 (e.g., ETT) and wash out more deadspace, thereby increasing CO₂ clearance and increasing oxygenation.

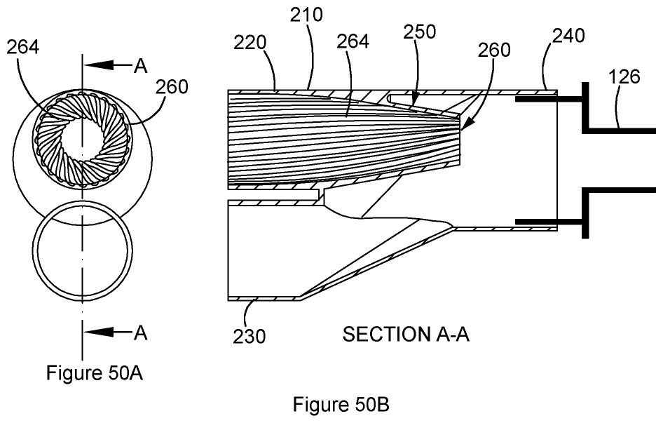

FIG. 50A is an end view which illustrates the jet nozzle having a helical structure 264 for producing rifling flow, and FIG. 50B is a cross-sectional view of FIG. 50A through the Section A-A, according to some embodiments of the invention. The helical structure 264 may be formed by internal wall structures or features such as ribs or grooves, to cause a rifling flow. For example, the spiralling structure 262 of FIG. 48, or a portion thereof, may be embedded into a wall of the nozzle to form a rifling or part rifling feature. In the shown connector 200 of FIGS. 50A-B, the wall structures are helical internal grooves 264 that result in a rifled flow. The grooves 264 impart a twist or spiral to the flow through the nozzle. Similar effects on RTF of the connector 200 and lower driving pressure of the system 100, as well as supporting larger diameter jet outlets 260 as described above in relation to FIG. 48 also apply to this embodiment. It should be appreciated that the rifling flow may be produced by other internal wall structures than having a helical structure 264, and that embodiments of the invention are not limited to having helical structures 264 to produce the rifling flow.

Connector with Coaxial Flow Paths

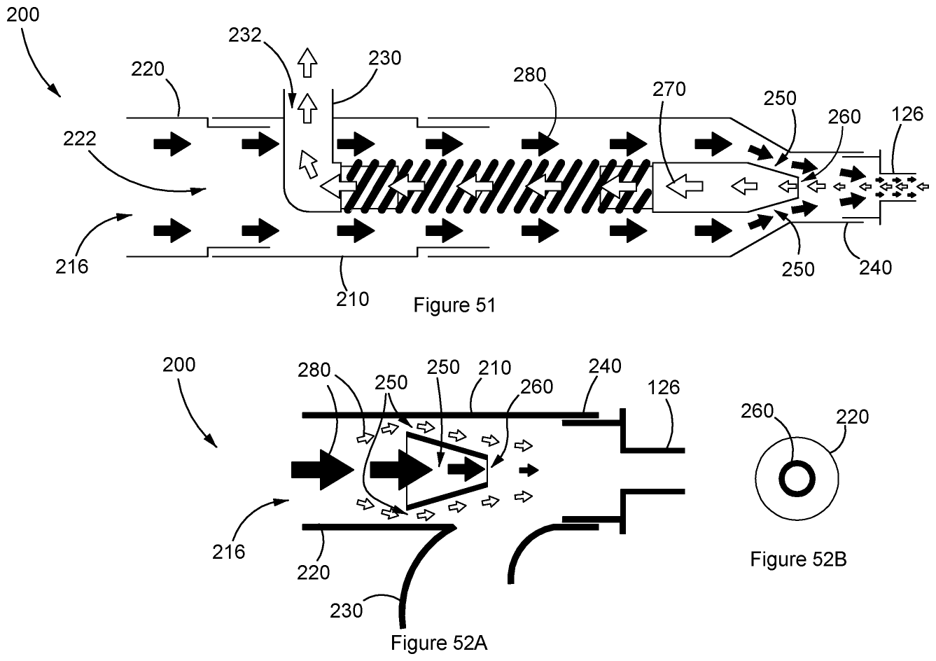

In some embodiments, the connector 200 is provided with coaxial inspiratory and expiratory flow paths 280, 270 as shown in the exemplary connector 200 of FIG. 51.

In FIG. 51, the expiratory flow path 270 is shown within the inspiratory flow path 280 and inspiratory flow is directed around the expiratory path 270 through the device port 240 into the invasive respiratory device 120, keeping close and/or attaching to the walls of the invasive respiratory device 120 in use. In this embodiment, the connector 200 includes an annular jet nozzle, however this nozzle is placed in the expiratory flow path 270. The flow constriction 250 on the inspiratory flow path 280 is defined between an outer wall provided by the tapering inlet channel 222 and the inner wall provided by the outer wall of the expiratory path 270, that is the annular jet nozzle. Expiratory flow exits through the centre of the invasive respiratory device 120 when coupled thereto via the centrally located expiratory path 270 within the inspiratory channel 222.

Connector with Flow Around Nozzle

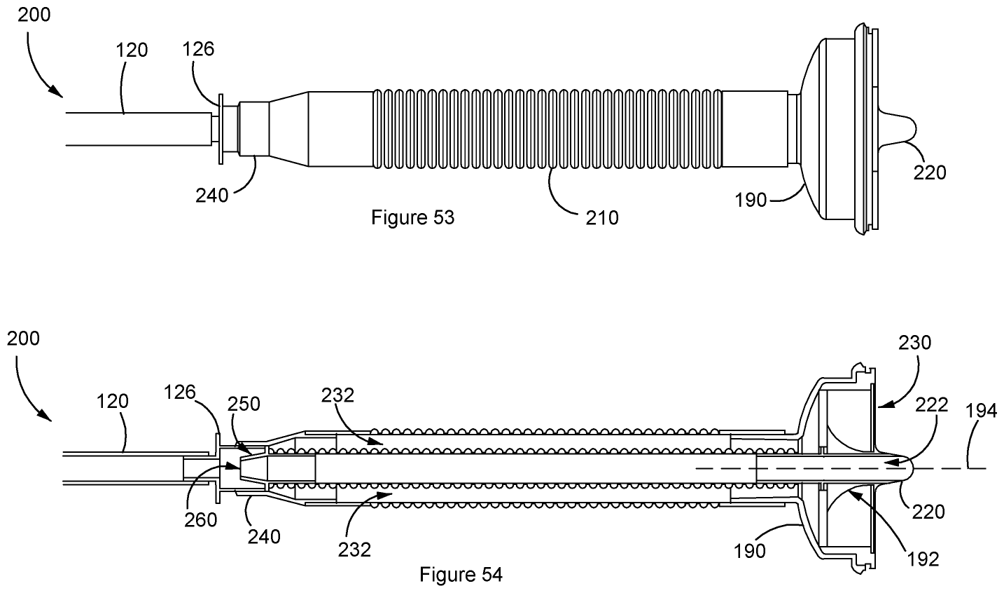

In some embodiments, the flow constriction 250 is formed by a tapered nozzle that allows inspiratory flow through and around the nozzle, as shown in the exemplary connector 200 of FIGS. 52A-B.

FIG. 52A is a schematic view showing another connector 200 with the jet nozzle positioned within the inspiratory flow path 280, enabling gas flow through and around the nozzle, according to some embodiments of the invention, as indicated by the arrows of the flow constriction 250. FIG. 52B shows an end view of the jet outlet 260 centrally aligned with the jet nozzle.

The connector 200 includes a flow constriction 250 being a nozzle positioned in the inspiratory flow path 280. This nozzle can be attached at various locations to the wall 210 of the connector 200, such as the inner wall 294 of the inlet channel 222 (see FIG. 29). The connector 200 can assist in reducing separation of flow that may occur with the single jet outlet 260. In this embodiment, inspiratory flow can go through the nozzle and outlet 260 but also around the nozzle. In an alternative embodiment, the area the flow passes through around the nozzle may have a constant cross-section. The effective RTF for these connectors may be reduced as the low-pressure region at the outlet 260 of the tapered nozzle can entrain gas from around the outside of the tapered nozzle.

Connector with Filter

In another inventive aspect, there is provided a connector 200 for coupling with an invasive respiratory device 120. The connector 200 includes a main body 210 having a gases port 220 for receiving a flow of gas from a flow source 110 at a selected flow rate, an outlet port 230 for outflow of gases from the main body 210, and a device port 240 couplable with the invasive respiratory device 120. The gases port 220 includes an inlet 216 and an outlet 260. The connector 200 is configured to receive the flow of gas from the flow source 110 via the inlet 216 of the gases port 220, and to deliver a jet flow of gas through the outlet 260 of the gases port 220. The connector 200 further includes a filter 190 couplable with the outlet port 230 for filtering the gases from the main body 210.

FIGS. 53 to 63 show embodiments of the connector 200 including a radial filter 190 couplable with the outlet port 230 for filtering the gases from the main body 210. The flow is directed and jetted to the invasive respiratory device 120 via an inspiratory flow path 280 through an inlet channel 222 in the centre 192 of the radial filter 190. The inlet channel 222 may preferably be positioned through a central axis 194 of the radial filter 190 as illustrated in these figures (see broken line representing central axis 194 as shown in FIGS. 54 and 59, for example). However, a person skilled in the art would appreciate that the inlet channel 222 may be off-centre relative to the central axis 194 and may be positioned near or about the centre 192 of the radial filter 190. Inspiratory flow is delivered through the centre 192 of the filter 190 through an inlet channel 222, jetted through the outlet 260 and into the invasive respiratory device 120. Expiratory flow from the invasive respiratory device 120 and around the jet outlet 260, travels through the expiratory path 270 via the filter 190 to atmosphere.

In the embodiments of FIGS. 53 to 63, the inspiratory flow path 280 and expiratory flow path 270 are coaxial. That is, the inlet channel 222 through which gases flow in the inspiratory flow path 280, and the outlet channel 232 through which gases flow in the expiratory flow path 270, are coaxial. The outlet channel 232 surrounds the inlet channel 222 in these embodiments. Notably, this is the different to the embodiment of FIG. 51, which illustrates that the inlet channel 222 at least partially surrounds the outlet channel 232/expiratory flow path 270.

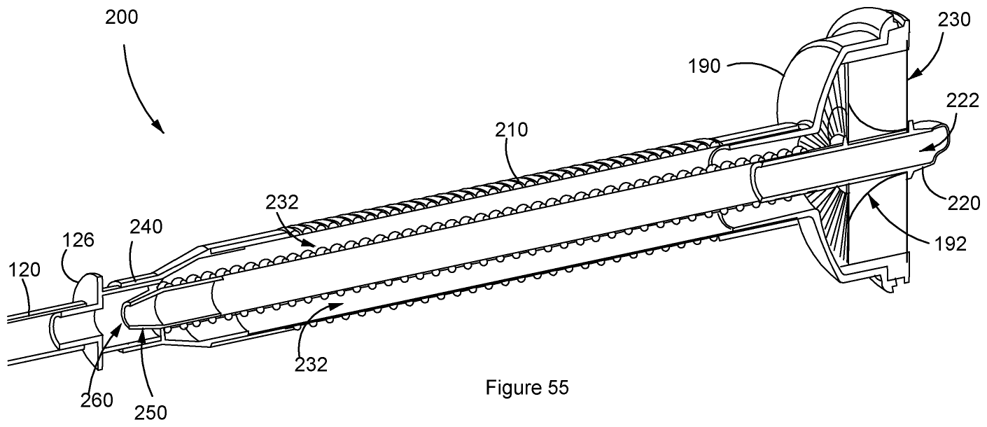

FIGS. 53 to 55 illustrate another embodiment of a connector 200 for coupling with an invasive respiratory device 120 via an adapter 126, showing coaxial inspiratory and expiratory flow paths. The connector 200 may optionally have a radial filter 190 on the expiratory flow path as illustrated. FIG. 54 illustrates a cross-sectional view and FIG. 55 illustrates a perspective sectional view, both of the connector 200 of FIG. 53, illustrating the jet nozzle and outlet 260, with the inlet channel 222 passing through a centre 192 of the filter 190. The inlet channel 222 is at least partly surrounded by the radial filter 190 near the end having the gases port 220. The expiratory flow path, which is defined between the device port 240 and the outlet port 230, extends from the invasive respiratory device 120, into the connector 200, and around the jet outlet 260 in the direction of the arrows 232 of the outlet channel. Thus, the outlet channel 232 surrounds the inlet channel 222 in this embodiment. In some embodiments, a filter may also be included on the inspiratory flow path (not shown). The filter could be the same filter component which extends across both the inspiratory and expiratory flow paths in the connector 200.

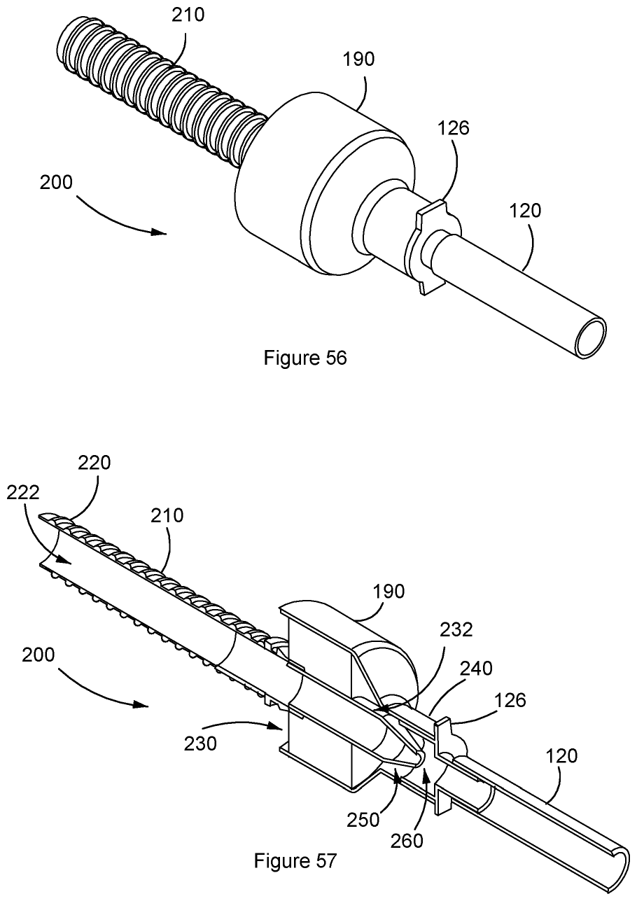

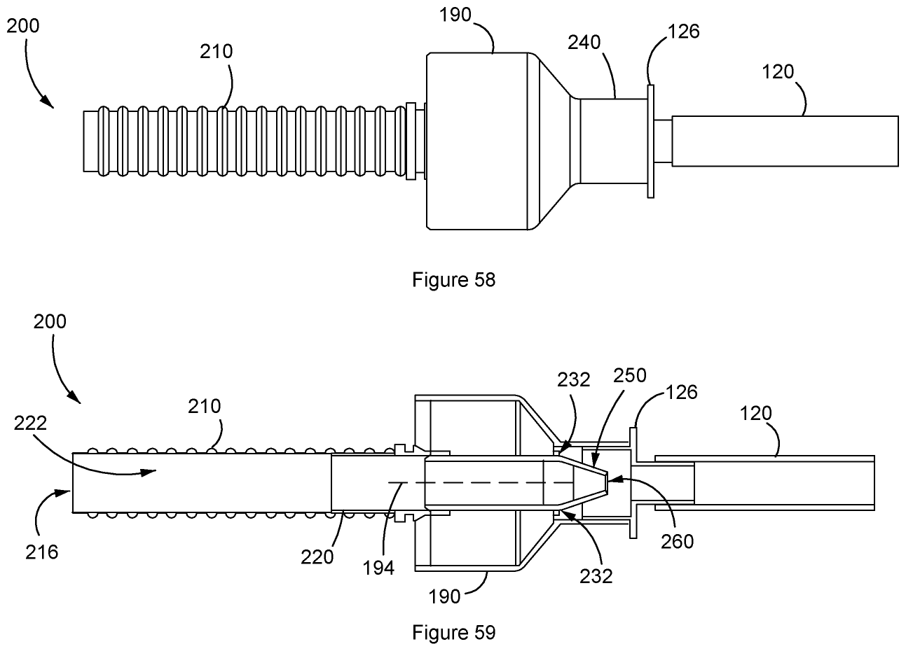

FIGS. 56 to 59 illustrate another connector 200 for coupling with an invasive respiratory device 120, showing coaxial inspiratory and expiratory flow paths and a radial filter 190 on the expiratory flow path in a perspective view (FIG. 56), perspective sectional view (FIG. 57), side view (FIG. 58) and cross-sectional view (FIG. 59). The connector 200 of FIGS. 56 to 59 has similar features to the connector 200 of FIGS. 53 to 55. The key difference is that the radial filter 190 is not located near the gases port 220 and instead is positioned surrounding part of the inlet channel 222 adjacent the flow constriction 250, being a tapered nozzle in this embodiment. Thus, the expiratory flow path, which passes around the jet outlet 260 indicated by the arrows 232 of the outlet channel (see FIG. 59), through the radial filter 190 and out to atmosphere, has a much shorter distance than the expiratory flow path of FIGS. 53 to 55.

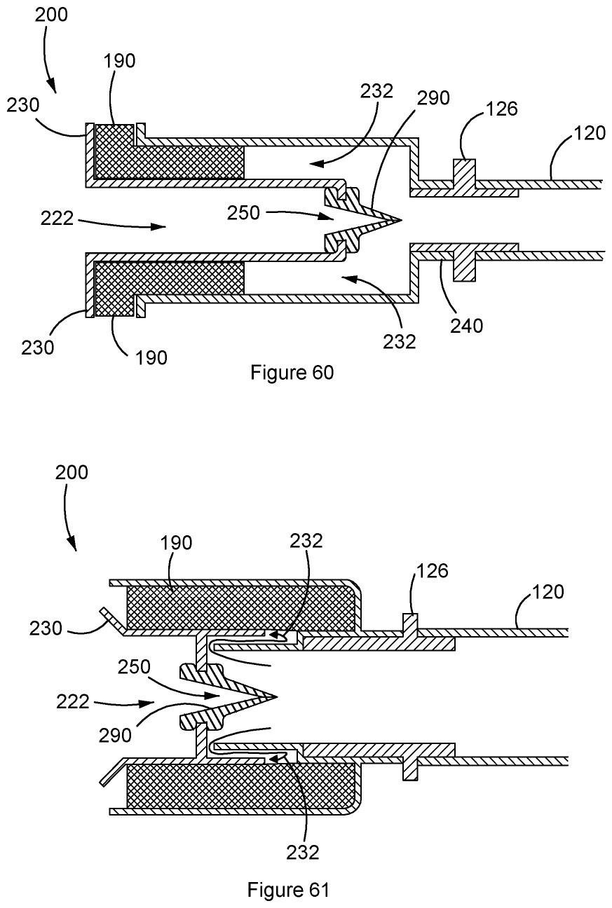

FIGS. 60 to 63 show embodiments of another connector 200 for coupling with an invasive respiratory device 120, showing a radial filter 190 couplable with the outlet port 230 for filtering the gases from the main body 210 and including a duckbill valve 290 on the inspiratory flow path 280. The duckbill valve 290 opens during inspiration allowing flow to jet into the invasive respiratory device 120 and can close or partially close on expiration, guiding flow through the filter 190. The amount that the valve 290 opens can be tailored by the flow rate of gases, for example, a higher flow rate will result in a larger opening of the valve 290. The amount that the valve 290 closes depends on an increase in pressure in the connector 200 due to expiratory flow, which will force the duckbill valve 290 to close or partially close, and the flow is vented through the radial filter 190 coupled with the outlet port 230. In some embodiments, when the duckbill valve 290 is closed, gas flow from the flow source 110 may be vented out of a valve, such as a pressure reducing valve (PRV) upstream of the duckbill valve 290 (not shown). The flow constriction 250 according to these embodiments of the invention may thus be provided by a valve mechanism instead of or in addition to a tapered nozzle and/or a plurality of apertures or openings. Although a duckbill valve 290 is shown in these embodiments, it will be appreciated that embodiments of the invention may include other forms of valves which provide similar functionality.

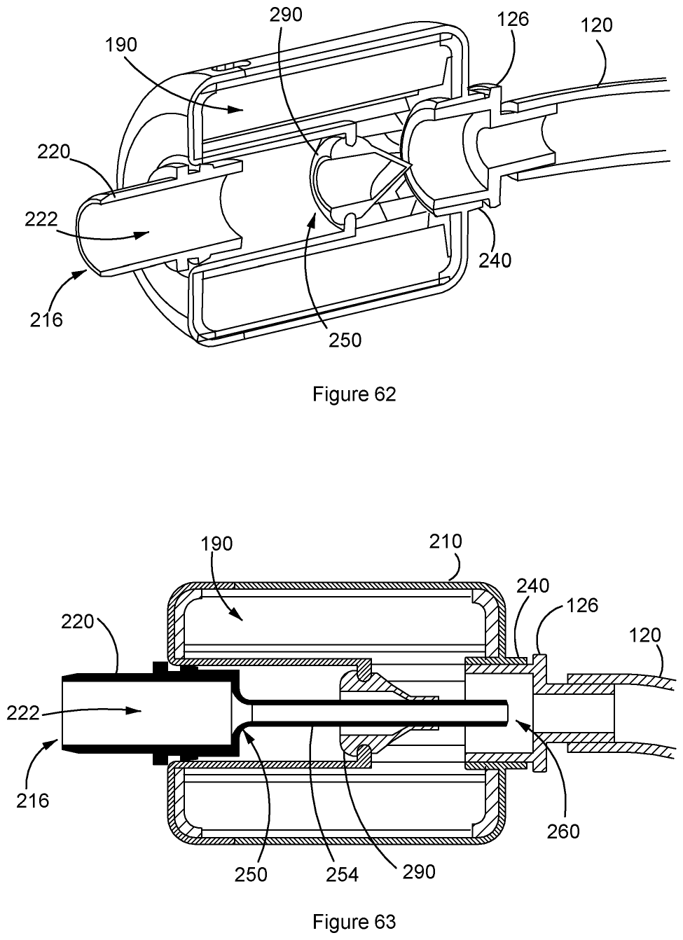

In the embodiment shown in FIG. 60, the outlet channel 232 and the inlet channel 222 are partly surrounded by the radial filter 190. The outlet channel 232 also surrounds the inlet channel 222. In contrast, in the embodiment shown in FIG. 61, the inlet channel 222 is entirely surrounded by the radial filter 190, whereas the outlet channel 232 is only partly surrounded. FIG. 62 illustrates a similar embodiment of the connector 200 of FIG. 61 in a perspective view.

FIG. 63 illustrates a cross-sectional view of another connector 200 including a radial filter 190 with a duckbill valve 290. In this embodiment, the flow constriction 250 is formed by a tapered nozzle having a constant diameter portion 254 adjacent the jet outlet 260. In this embodiment, the duckbill valve 290 allows the nozzle with constant diameter portion 254 to penetrate the valve 290 and provide jet flow through the valve opening in use. When the tapered nozzle is removed, the duckbill valve 290 closes and flow into the main body 210 of the connector 200 is prevented.

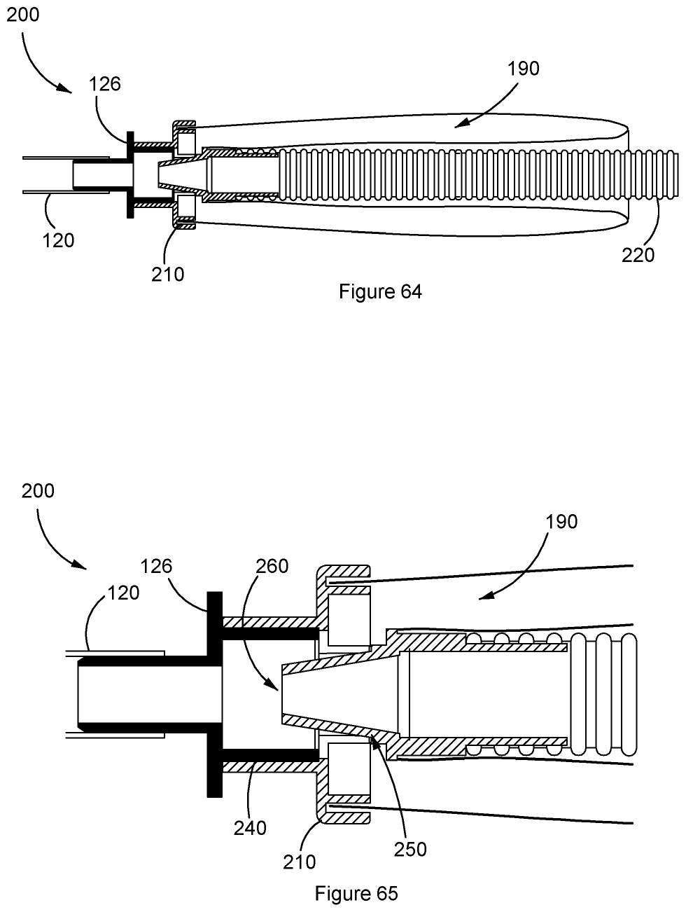

FIGS. 64 and 65 illustrate another connector 200 having a bag or receptable filter 190 on the expiratory flow path, according to some embodiments of the invention, with an enlarged view of the jet nozzle illustrated in FIG. 65. The filter 190 substantially surrounds the inlet channel 222 of the connector 200. In this embodiment, the inspiratory and expiratory flow paths are substantially coaxial. A bag or receptacle filter 190 can advantageously have a lower RTF than other filters.

Connector with Variable Resistance to Expiratory Flow

According to another inventive aspect, one or more connectors 200 of the present disclosure may be provided with a variable aperture for adjusting resistance to flow of gases exiting the connector through the outlet port 230. The features providing the adjustable aperture may be formed in the main connector body 210 or in a connector body extension (not shown) which couples with the outlet port 230 e.g. by friction fit or threaded coupling. In use, the variable aperture may be used to control resistance to flow of gases exiting through the outlet port 230 (or extended outlet port) which in turn gives rise to different patient pressures achieved within the patient 300 during provision of respiratory support.

Adjusting resistance to flow of gases exiting the outlet port 230 may be useful in some embodiments. For example, increasing resistance to flow through the outlet port 230 by reducing the size of the variable aperture may increase patient pressure which in turn, may increase CO₂ clearance. Alternatively/additionally, increasing resistance to flow by decreasing the size of the variable aperture may be desirable to increase Positive End Expiratory Pressure (PEEP).

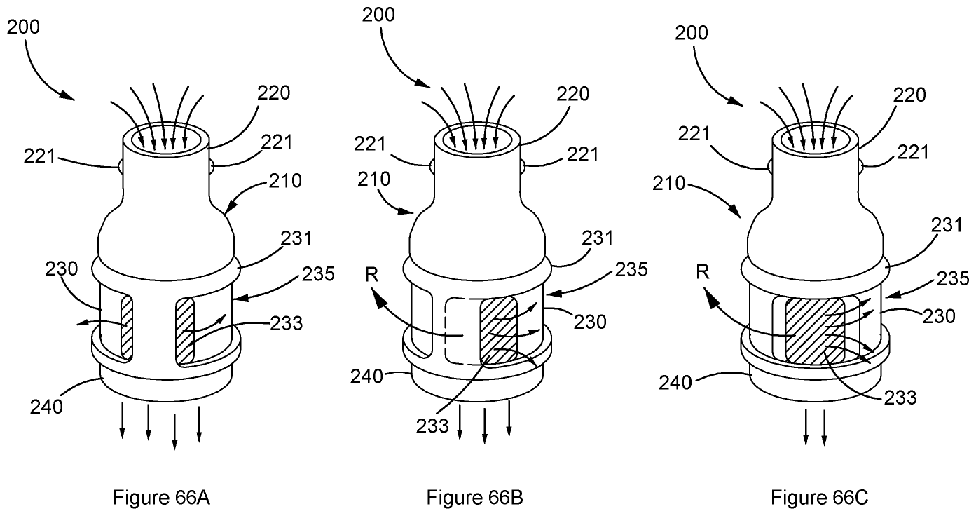

In one example according to FIGS. 66A to 66C, connector body 210 has similar features to connectors described elsewhere herein including gases port 220 which receives respiratory gases from a flow source via a conduit (not shown) which may be coupled with the connector 200 using e.g. friction fit projections 221, and device port 240 configured to couple with an invasive patient interface 120 (not shown). Gases exit connector body 210 through outlet port 230 which has a variable aperture defined by first opening 233 which is provided in a wall portion defining the outlet port 230 and a moveable collar 231. Collar 231 is arranged around at least part of the wall portion defining the outlet port 230. The hashed region represents first opening 233 and the stylistic arrows represent the direction of flow of gases when in use. The collar 231 may be a complete annulus or an incomplete annulus such as a broken ring or collar. The collar 231 has a second opening 235. Movement of collar 231 adjusts an amount of overlap between the first opening 233 and the second opening 235 to vary the extent to which the variable aperture, through which gases exit connector 200, is open. When collar 231 is rotated so that the solid part of the collar is arranged over first opening 233, the variable aperture is at its smallest opening size providing maximum resistance to flow to gases exiting the connector 200 through outlet port 230. When collar 231 is rotated so that there is less overlap between first opening 233 and second opening 235, the variable aperture is larger, reducing the resistance to flow.

FIGS. 66A to 66C show collar 231 rotated to different positions which provide different variable aperture configurations. FIG. 66A shows two small apertures providing maximum resistance to flow available with this collar arrangement. It is to be noted that the embodiment shown provides a safety feature in that the collar structure 231 does not allow the first opening 233 of the outlet port 230 to be fully covered. In FIG. 66B collar 231 has been rotated clockwise relative to FIG. 66A as shown by arrow R, such that part of the solid section of collar 231 is occluding first opening 233 which reduces the resistance to flow of gases exiting the outlet port 230 relative to FIG. 66A. In FIG. 66C collar 231 has been further rotated clockwise in the direction of arrow R such that the variable aperture in the most open position providing the lowest resistance to flow of gases exiting the outlet port 230. It is to be understood that reducing the resistance to flow of gases exiting the outlet port 230 reduces the total resistance to flow of the connector 200, which is represented as the pressure loss between the gases port 220 which receives gases from the flow source, and the outlet port 230. Therefore, assuming other connector and flow parameters remain the same, increasing the size of the variable aperture reduces the resistance to flow which in turn reduces the patient pressure. Conversely, assuming other connector and flow parameters remain the same, decreasing the size of the variable aperture increases the resistance to flow which in turn increases the patient pressure.

Although FIGS. 66A to 66C show collar 231 being rotationally movable relative to the wall portion of the outlet port 230, it is to be understood that a ring or other annular-type collar may be translated along the wall portion to achieve a varying aperture size. Furthermore, it is to be understood that the second opening 235 which is formed in the collar 231 may take the form of a single opening of any shape or size that can be manufactured into the collar, or a plurality of openings or holes which may be variably aligned with the first opening by sliding the collar 231 along or around the outlet port 230.

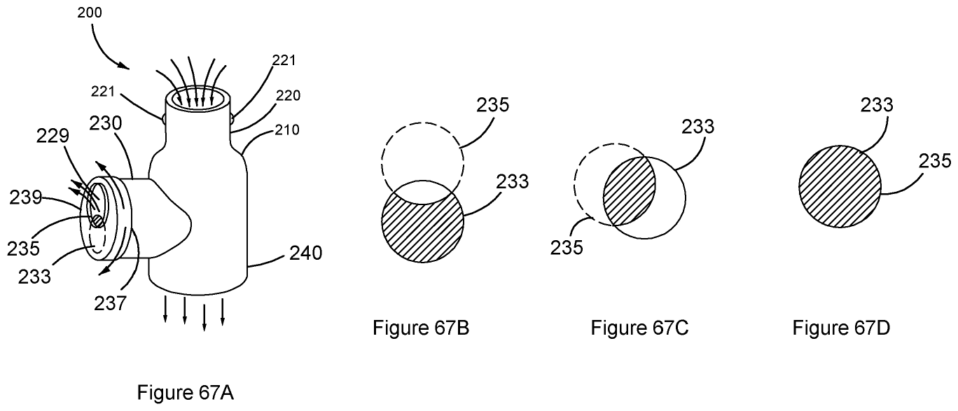

In another example according to FIGS. 67A to 67D, connector body 210 has similar features to connectors described elsewhere herein including gases port 220 which receives respiratory gases from a flow source (not shown) and device port 240 configured to couple with an invasive patient interface 120 (not shown). In the embodiment shown, the outlet port 230 is a side port of connector 200 which provides a structure to which cap 237 may be applied to provide a variable aperture. In some embodiments the cap 237 is permanently formed over the opening of outlet side port 230 and in other embodiments, the cap 237 is removably applied by helical thread, friction fit or the like. Cap 237 has a first member 229 with a first opening 233 (represented by a hashed region) and a second member 239 with a second opening 235. One of the first and second members 229, 239 is moveable relative to the other member to alter the degree of overlap between the first and second openings 233, 235 in those members. As can be seen in FIG. 67A, the first and second members 229, 239 are circular discs with respective openings offset from center. Relative rotational movement between the first member 229 and second member 239 varies an amount of overlap between the first and second openings 233, 235 to define and alter the variable aperture. The stylistic arrows represent the direction of flow of gases.

In FIG. 67A, first member 229 is almost entirely concealed beneath second member 239 (except for the part that can be seen through second opening 235). In some embodiments, first member 229 be permanently applied over the opening of outlet side port 230, or fixed in place as part of a cap 237 which is applied over the opening of the outlet side port as described previously. Thus first member 229 is typically stationary in use, with the second member 239 rotationally moveable relative to the first member.

FIGS. 67B to 67D are schematic illustrations showing relative movement of second opening 235 (represented by broken lines) relative to stationary first opening 233. The first and second members 229, 239 in which the openings are provided have been omitted for simplicity. The variable aperture formed by the overlapping openings is represented by hashed lines. In FIG. 67B, there is a small overlap between the first and second openings 233, 235 providing a small aperture and relatively high resistance to flow of gases exiting the outlet side port 230. In FIG. 67C, there is a larger overlap between the first and second openings 233, 235, providing a larger aperture and moderate resistance to flow of gases exiting the outlet side port 230. In FIG. 67D, the first and second openings 233, 235 overlap in their entirety providing the maximum available aperture size and lowest resistance to flow of gases exiting the outlet side port 230. It is to be noted that the embodiment shown provides a safety feature in that the arrangement of the first and second openings 233, 235 in first and second members 229, 239 does not allow the outlet side port 230 to be fully closed.

System with Connectors

According to another inventive aspect, there is provided a system 100 for providing respiratory support to a subject 300. The system 100 includes a flow source 110 for providing a gas at a selected flow rate, an invasive respiratory device 120 couplable with an airway of the subject 300, and the connector 200 according to any one of the inventive aspects, or any combinations of the inventive aspects or embodiments as described herein. The system 100 may include any one of the inventive aspects, or any combinations of the inventive aspects with features of embodiments described in connection with the system 100 of FIG. 1.

In some embodiments, the system 100 as described herein may also include an optional pressure relief valve, or a sputum catcher. This may be beneficial when the connector 200 is used with a tracheostomy tube for the invasive respiratory device 120.

In some embodiments, the system 100 as described herein may also include a tracheostomy guard, such as described in US20170049982 which is incorporated herein by reference.

Kit with Connector

According to another inventive aspect, there is provided a kit 500 for a system 100 for providing respiratory support to a subject 300. The kit 500 includes the connector 200 according to any one of the inventive aspects, or any combinations of the inventive aspects with features of the embodiments as described herein. The kit 500 also includes at least one of a filter 190 couplable with the outlet port 230 of the connector 200, an invasive respiratory device 120 couplable with the connector 200, and an adapter 126 couplable to the device port 140 of the connector 200 for coupling an invasive respiratory device 120 with the connector 200.

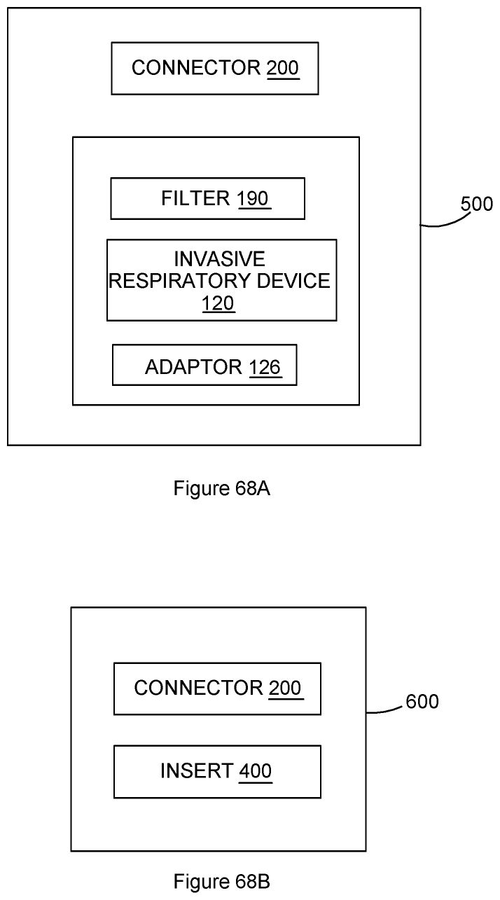

FIG. 68A is a schematic diagram showing components of the kit 500 for a system 100 for providing respiratory support to a subject 300, according to some embodiments of the invention. The kit 500 includes the connector 200 and optionally, at least one of the filter 190, the invasive respiratory device 120, and the adapter 126 as indicated by the broken lines.

In some embodiments (not shown), the kit 500 may further include an interface conduit 180 connectable between the inlet 216 of the gases port 220 of the connector 200 and a flow source 110 for providing fluid communication (for example, see interface conduit 180 and flow source 110 described with reference to FIG. 1). The kit 500 may further include a filter 170 which is couplable between the inlet 216 of the gases port 220 of the connector 200 and the flow source 110 (see also the filter 170 described with reference to FIG. 1).

In some embodiments (not shown), the kit 500 may further including a humidifier 140 for conditioning gas flow provided by a flow source 110 to a selected temperature and/or humidity suitable for delivery to a patient 300 (see also FIG. 1 and related description). The kit 500 may optionally include the humidifier 140 (not shown). In some embodiments, the humidifier 140 includes a humidification chamber 142 and humidification base unit 150 as shown in FIG. 1. The kit 500 may optionally include a humidification chamber 142 and/or base unit 150. In some embodiments (not shown), the kit 500 may further include a conduit 130 connectable between the flow source 110 and the humidifier 140, and/or an inspiratory conduit 160 connectable between the humidifier 140 and the gases port 220 for flow communication.

Kit with Connector and Insert

According to another inventive aspect, there is provided another kit 600 for a system 200 for providing respiratory support to a subject. The kit 600 includes the connector 200 according to any one of the inventive aspects, or any combinations of the inventive aspects with features of embodiments as described herein. The kit 600 also includes the insert 400 according to the inventive aspect or any combinations of the inventive aspect with features of embodiments as described herein.

FIG. 68B is a schematic diagram showing components of the kit 600 according to some embodiments of the invention. The kit 600 includes the connector 200 and the insert 400. In some embodiments (not shown), the kit 600 further includes at least one of a filter 190 couplable with the outlet port 230 of the connector 200, an invasive respiratory device 120 couplable with the connector 200, and an adapter 126 couplable to the device port 240 of the connector 200 for coupling an invasive respiratory device 120 to the connector 200.

In some embodiments (not shown), the kit 600 may include one or more inserts 400. Preferably, the kit 600 includes at least two inserts 400 which have different lengths and/or locating features in order to alter the desired distance between the outlet 260 and a distal end portion 122 of an invasive respiratory device 120 when coupled to the connector 200. The inserts 400 may be selected for the kit 600 based on desired outcomes for providing respiratory support to the subject 300.

In some embodiments (not shown), the kit 600 may further include an interface conduit 180 connectable between the inlet 216 of the gases port 220 of the connector 200 and a flow source 110 for providing fluid communication (for example, see interface conduit 180 and flow source 110 described with reference to FIG. 1). The kit 600 may further include a filter 170 (not shown) which is couplable between the inlet 216 of the gases port 220 of the connector 200 and the flow source 110 (see also the filter 170 described with reference to FIG. 1).

In some embodiments (not shown), the kit 600 may further including a humidifier 140 for conditioning gas flow provided by a flow source 110 to a selected temperature and/or humidity suitable for delivery to a patient 300 (see also FIG. 1). The kit 600 may optionally include the humidifier 140 (not shown). In some embodiments, the humidifier 140 includes a humidification chamber 142 and humidification base unit 150 as shown in FIG. 1. The kit 600 may optionally include a humidification chamber 142 and/or base unit 150. In some embodiments (not shown), the kit 600 may further include a conduit 130 connectable between the flow source 110 and the humidifier 140, and/or an inspiratory conduit 160 connectable between the humidifier 140 and the inlet of the gases port 220 for flow communication.

### EXAMPLES

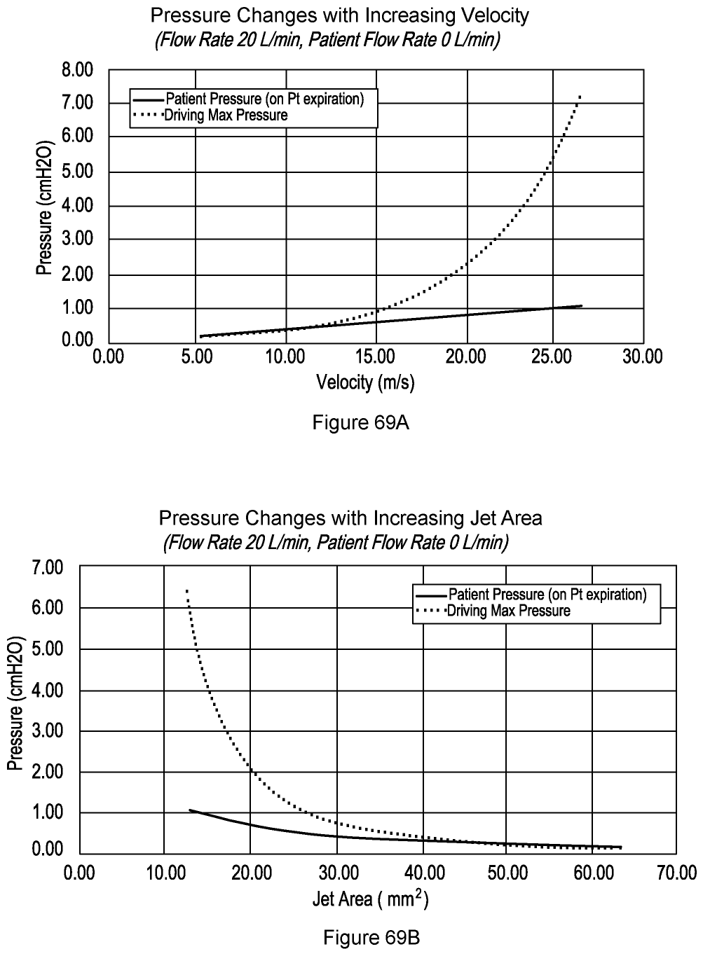

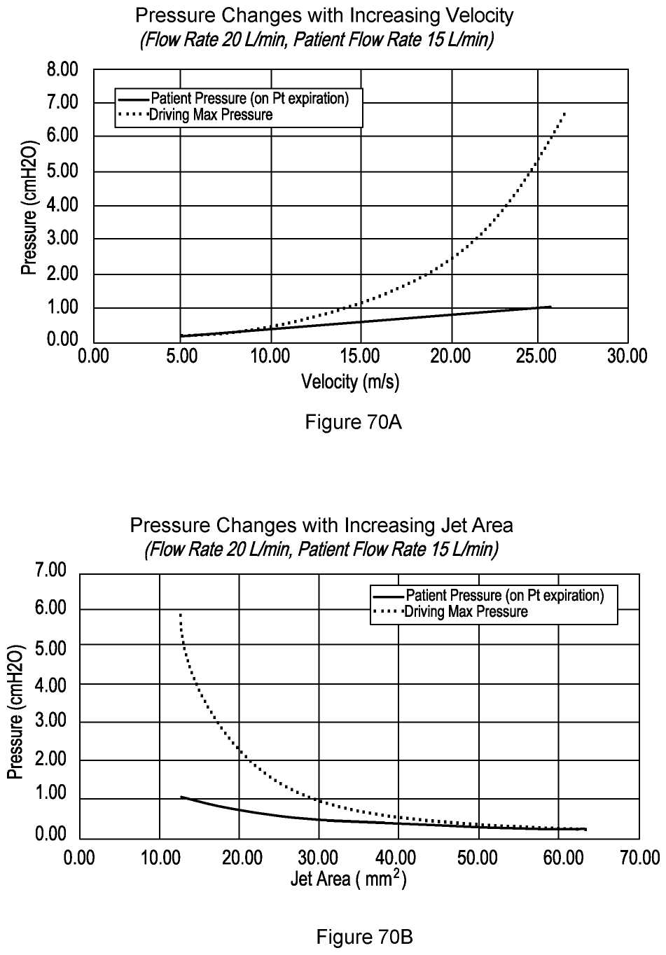

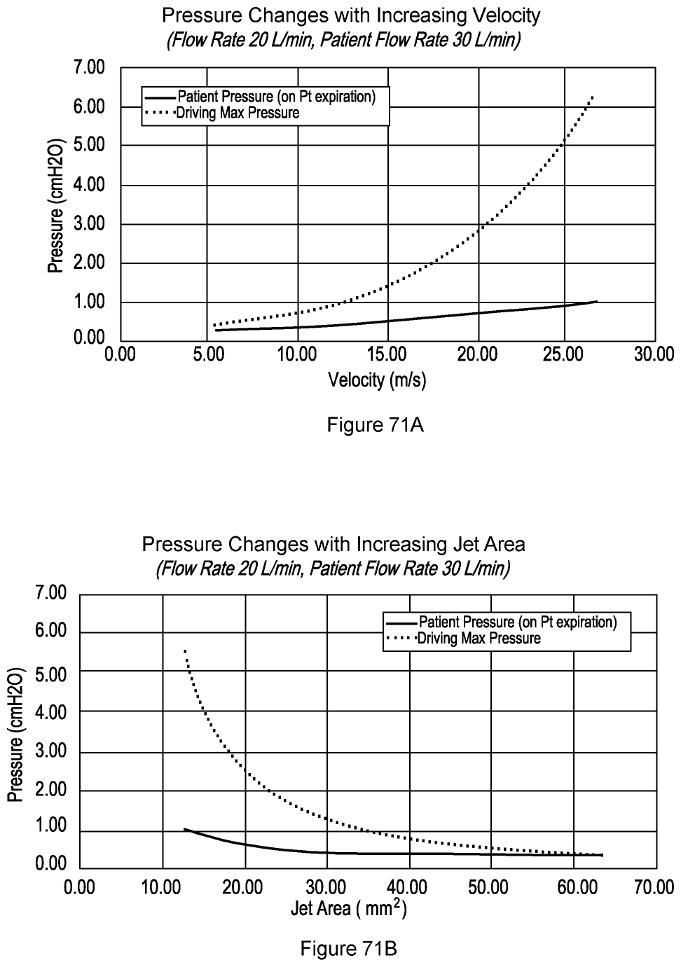

Examples illustrating applications of embodiments of the invention will now be described. The examples are supplied to provide context and explain features and advantages of the invention and are not limiting on the scope of the invention as defined in the claims. FIGS. 69A-B to 83 illustrate charts showing experimental results of the system 100 employing connectors 200 according to various embodiments of the invention.

### Example 1—Velocity and Diameter Study

FIGS. 69A-B to 78A-B relate to an experimental study concerning the outlet 260 of the flow constriction 250 and altering parameters including the velocity of the jet flow (‘A’ charts) and diameter A of the outlet 260 (‘B’ charts). It was found that the patient pressure and driving pressure generally increased with increasing flow velocity and decreased with increasing jet area.

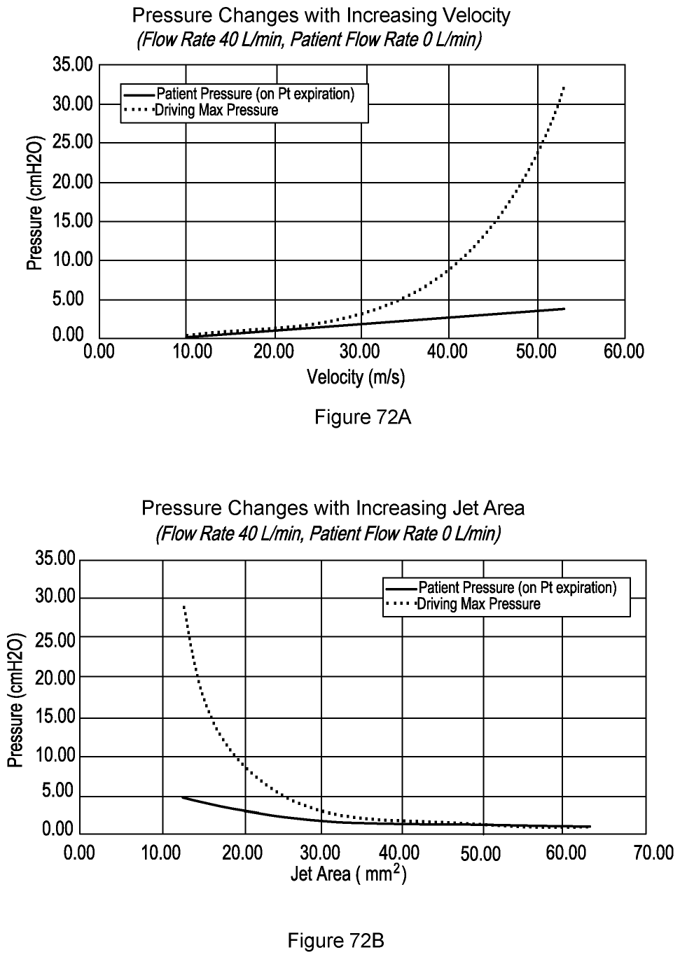

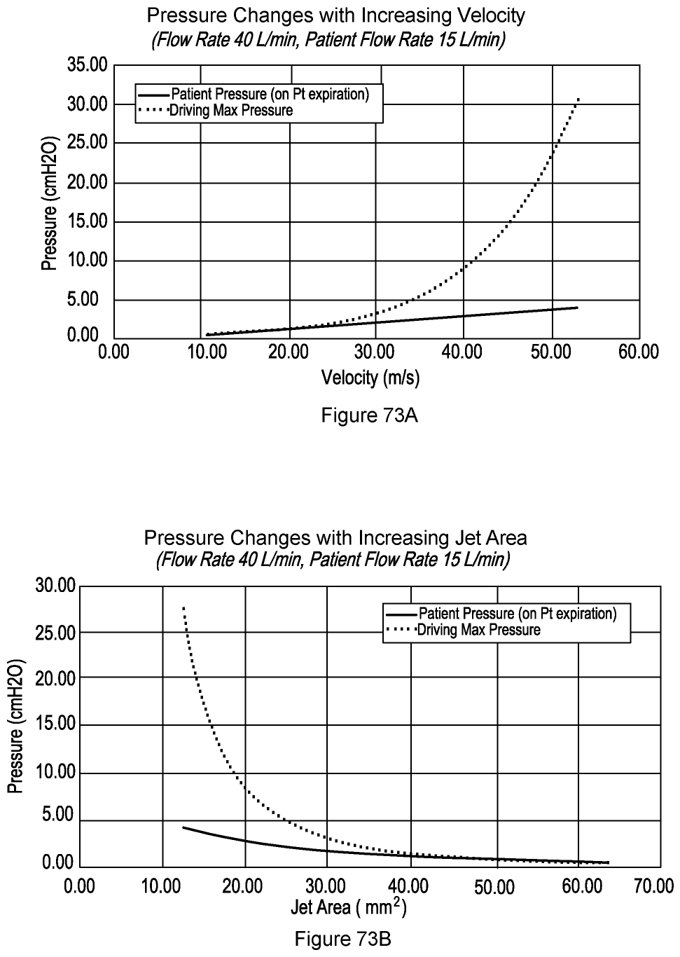

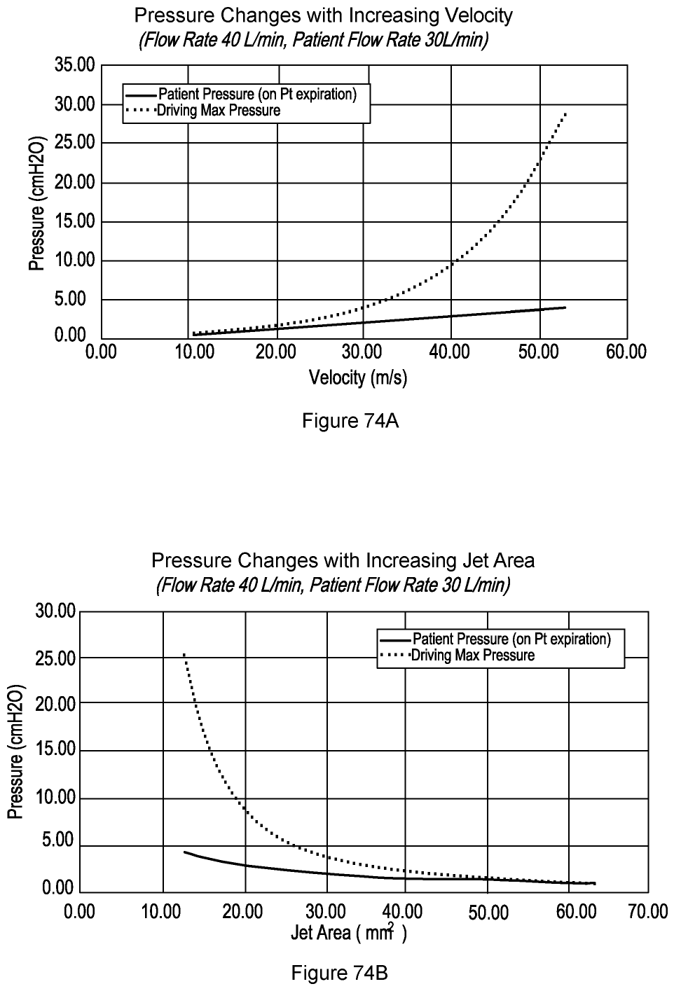

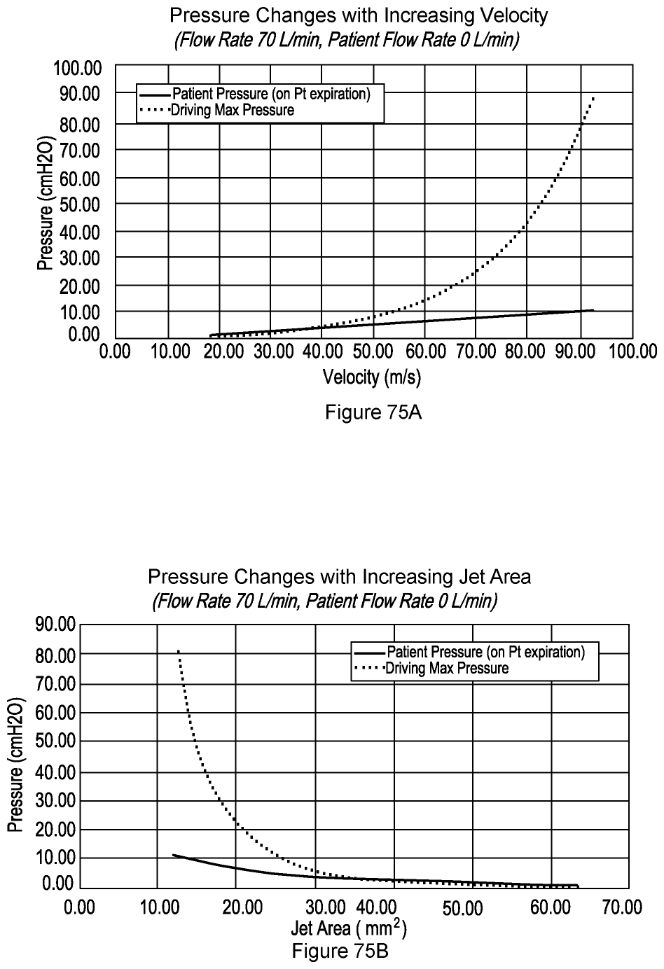

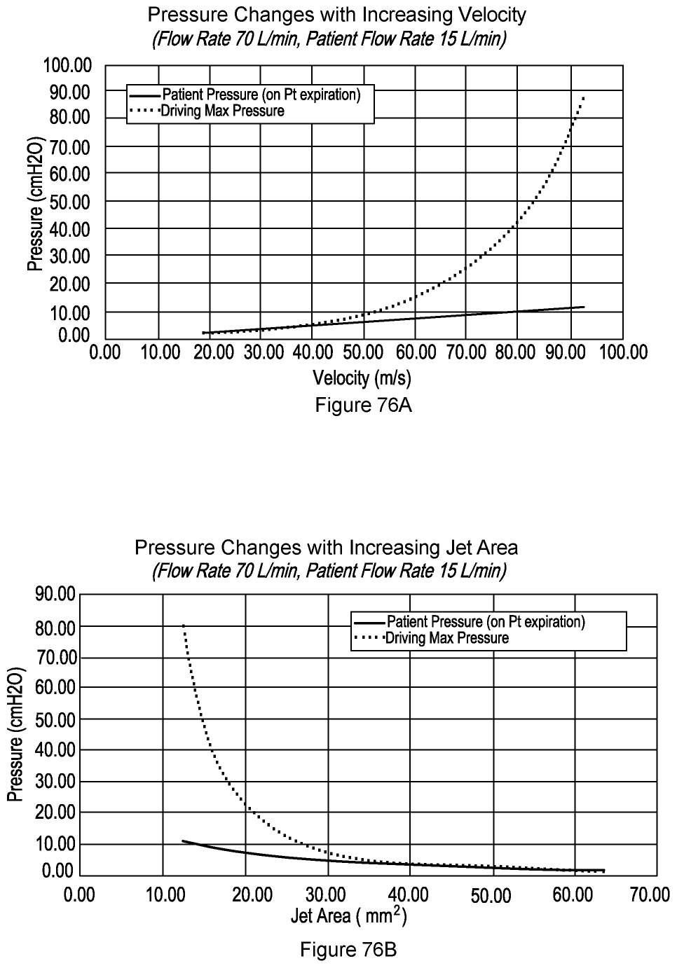

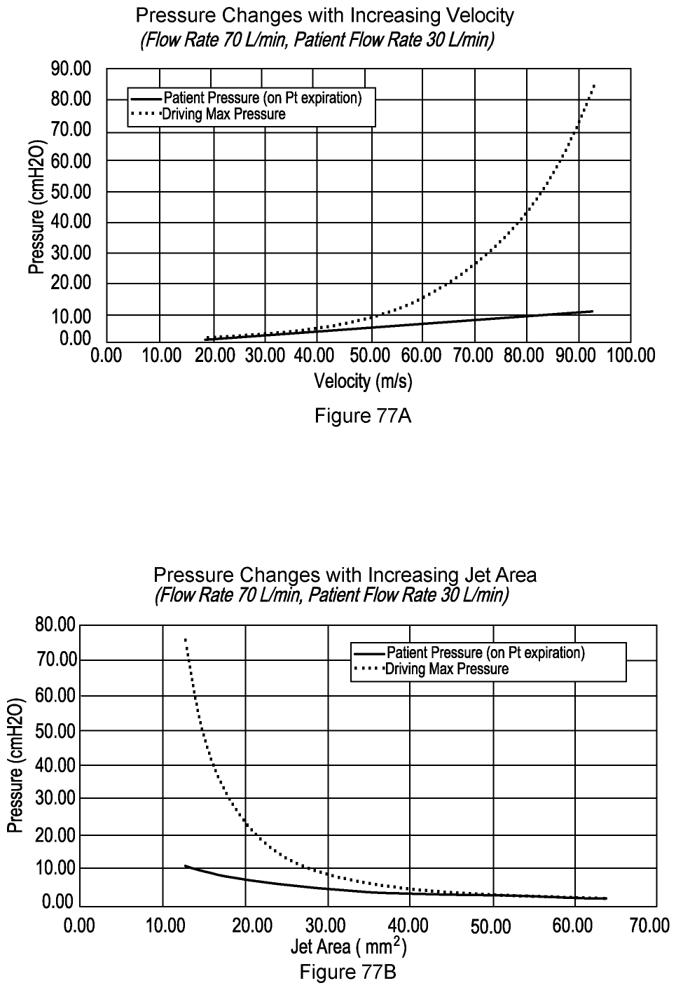

FIGS. 69A-B to 77A-B illustrate charts showing pressure changes with increasing velocity of the jet flow and increasing cross-sectional area of the jet for a selected flow rate of 20 L/min (FIGS. 69A-B, 70A-B and 71A-B), 40 L/min (FIGS. 72A-B, 73A-B and 74A-B) and 70 L/min (FIGS. 75A-B, 76A-B and 77A-B). The systems were also tested with generating patient flow rates of 0 L/min (apnoeic patient, FIGS. 69A-B, 71A-B and 75A-B), 15 L/min (normal breathing, FIGS. 70A-B, 73A-B and 76A-B) and 30 L/min (deep breathing, FIGS. 71A-B, 74A-B and 77A-B). As can be observed in FIGS. 69A-B to 77A-B, for all flow rates tested (20 L/min, 40 L/min and 70 L/min) and for all patient flow rates (0 L/min, 15 L/min and 30 L/min), the patient pressure and the driving pressure generally increased with increasing velocity of the jet flow along similar trend lines and decreased along similar trend lines with increasing jet area.

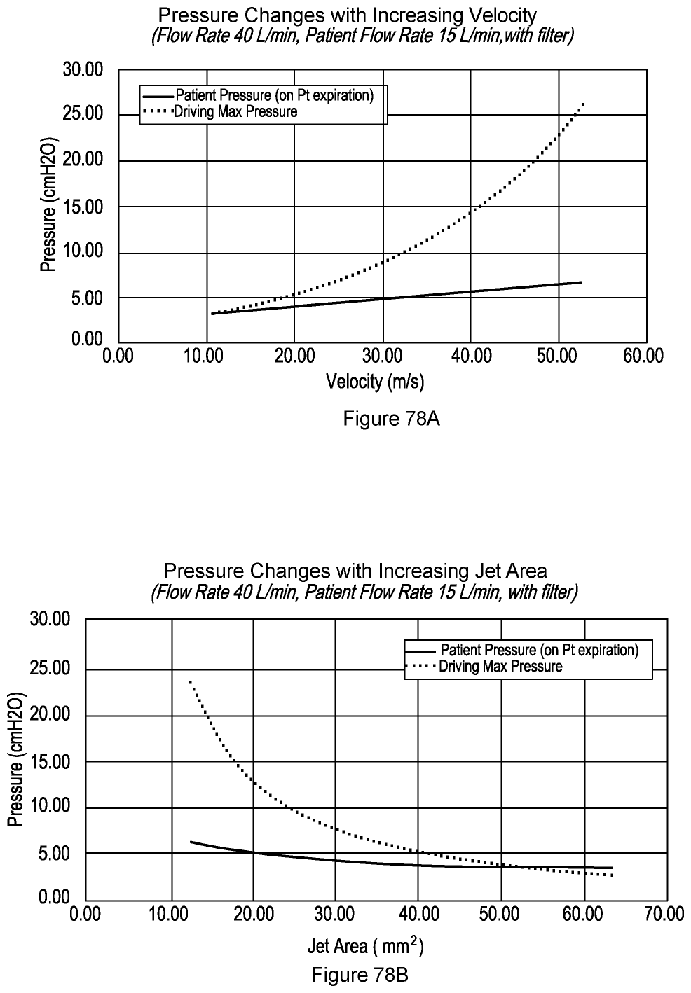

The systems tested in respect of the charts shown in FIGS. 69A-B to 77A-B did not include a filter 190 on the outlet port 230. For comparison, a system 100 was tested in which a filter 190 was included on the outlet port 230 of the connector 200. FIGS. 78A-B illustrate a chart showing pressure changes with increasing velocity of the jet flow (FIG. 78A) and increasing cross-sectional area of the jet (FIG. 78B) for a selected flow rate of 40 L/min and a patient flow rate of 15 L/min (normal breathing). It can also be observed that the patient pressure and driving pressure increased with increasing velocity of the jet flow and decreased with increasing jet area.

### Example 2—Minimum Expiration Area Study

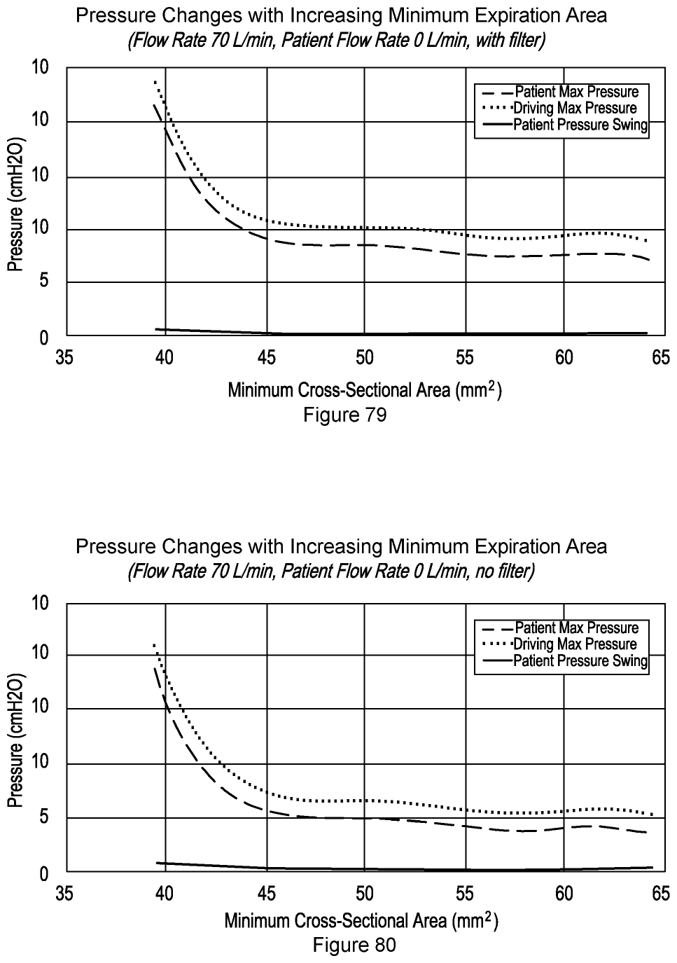

FIGS. 79 and 80 relate to an experimental study concerning the expiratory flow path 270 and altering parameters including the minimum diameter or minimum expiration area C of the expiratory flow path 270 for various connectors 100. It was found that as the expiration area is increased, the patient pressure and driving pressure decreased.

FIGS. 79 and 80 illustrate charts showing pressure changes with increasing minimum cross-sectional area C of the expiratory flow path 270 for a selected flow rate of 70 L/min and patient flow rate of 0 L/min (apnoeic) with a filter 190 on the outlet port 230 (FIG. 79) and without a filter (FIG. 80). It was shown that the patient pressure and driving pressure decreased for both examples as the minimum cross-sectional area of the expiratory path increased, and that this was consistent with embodiments of the connector 200 with and without a filter 190.

### Example 3—Jet Depth Study

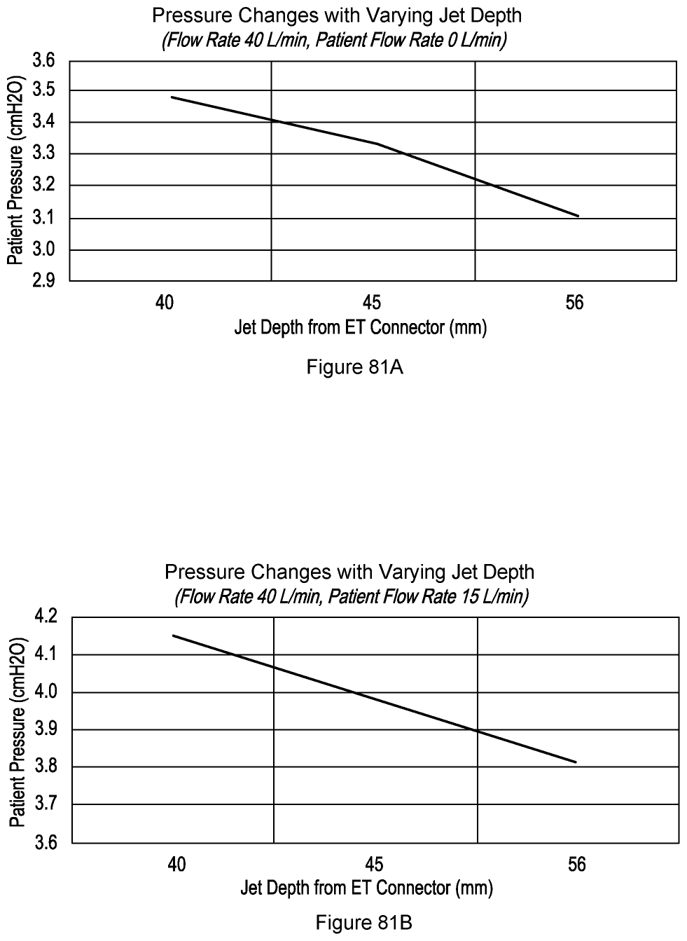

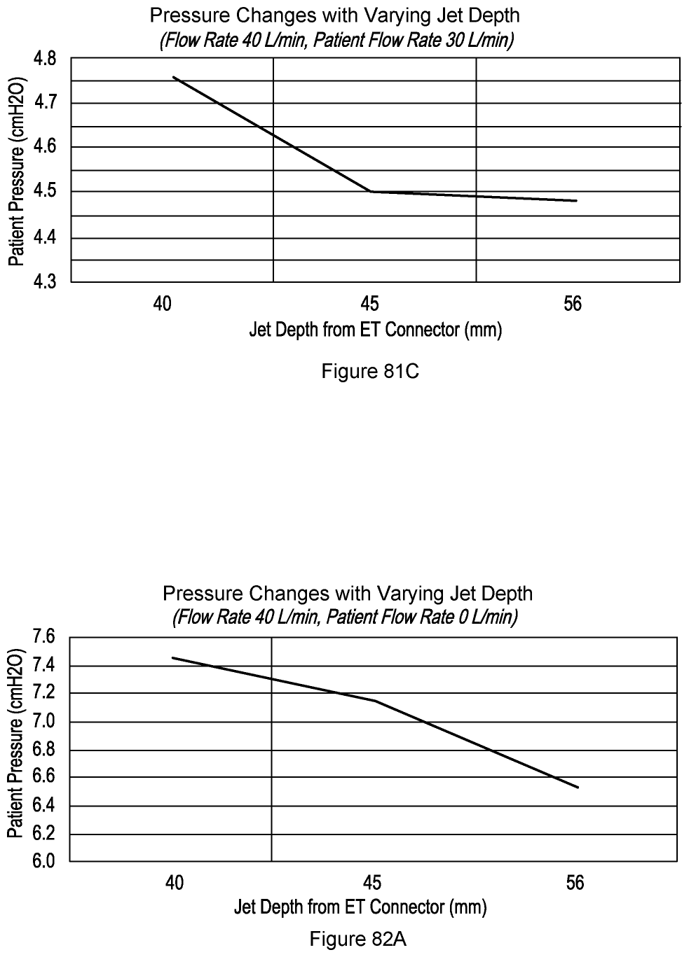

FIGS. 81A-C and 82A-C relate to an experimental study concerning the flow constriction 250 and altering parameters including a desired distance B (jet depth) from the jet outlet 260 to the device port 240, more particularly, a distal end portion 122 of the invasive respiratory device 120 (e.g., the ETT). It was found that the patient pressure decreased with increasing distance of the jet outlet 260 from the device port 240 coupled to the ETT connector or adapter 126 for the invasive respiratory device 120 (e.g., the ETT).

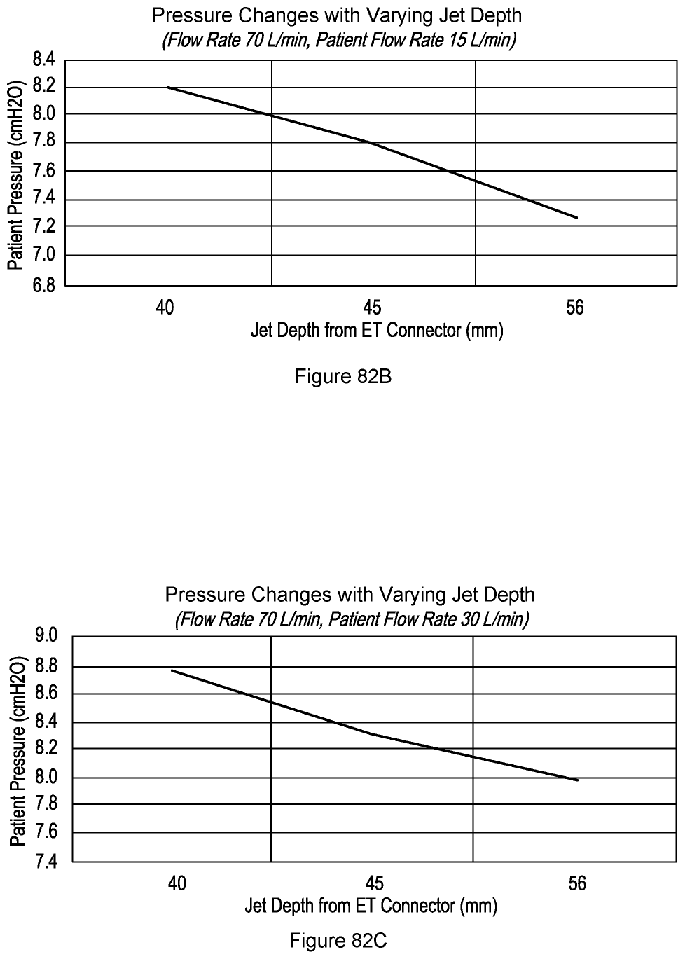

FIGS. 81A-C and 82A-C illustrate charts showing pressure changes with varying jet depth for a selected flow rate of 40 L/min (FIGS. 81A-C) and 70 L/min (FIGS. 82A-C). The systems were also tested with generating patient flow rates of 0 L/min (apnoeic patient, FIGS. 81A and 82A), 15 L/min (normal breathing, FIGS. 81B and 82B) and 30 L/min (deep breathing, FIGS. 81C and 82C). As can be observed in FIGS. 81A-C and 82A-C, for all flow rates tested (40 L/min and 70 L/min) and for all patient flow rates (0 L/min, 15 L/min and 30 L/min), the patient pressure generally decreased along similar trend lines with increasing distance of the jet outlet 260 from the adapter 126/device port 240.

### Example 4—Expiration Resistance Study

An example of the advantageous effect of lower expiration resistance in relation to the connector 200 of embodiments of the invention can be observed in FIG. 83.

FIG. 83 illustrates a chart showing pressure changes (swing) during inspiration and expiration for a subject using the system of FIG. 1, with a connector 200 having a high expiration resistance and a connector 200 having a low expiration resistance, according to some embodiments of the invention. The ‘small swing’ or broken line in the chart is indicative of the system 100 of FIG. 1 using the connector 200 having a low expiration resistance, whereas the ‘large swing’ or solid line in the chart is indicative of the system 100 of FIG. 1 using the connector 200 having a high expiration resistance. The ‘swing’ refers to the amplitude of pressure change between peak inspiration and peak expiration as indicated on the chart. The amplitude of pressure denoted by numeral 1 of the ‘small swing’ is less than the amplitude of pressure denoted by numeral 2 of the ‘large swing’.

In effect, the use of the inventive connector 200 having low expiration resistance may reduce the pressure swing or amplitude, lowering the pressure swing of the patient's breathing in use. The main benefit of reducing this pressure swing is that the inspiration pressure may be higher for a given expiration pressure, and this may make it easier for the patient to inspire and breathe using the inventive system 100 with inventive connector 200, according to embodiments of the invention.

### Advantages of the Invention

Embodiments of the invention aim to effectively deliver high flow respiratory support invasively by employing a jet flow of gas into an invasive respiratory device (such as an ETT) when in use. The jet flow may be produced by an inventive connector that in some embodiments includes a flow constriction and being located in the inspiratory flow path. The connector may be configured to jet flow of the gas through an outlet and into the ETT. The inventive connector includes various parameters that may be tuned to achieve certain beneficial characteristics. For example, the inventive system may improve respiratory support to a subject by generating a patient pressure that maintains a patent patient airway for a given range of flow rates, and/or providing a low resistance to flow (RTF) in the system, and/or requiring a lower driving pressure in the system. Parameters of the inventive connector may be tuned to address one or more of these system characteristics for providing high flow respiratory support invasively to a patient.

It is to be understood that various modifications, additions and/or alternatives may be made to the parts previously described without departing from the ambit of the present invention as defined in the claims appended hereto.

The invention may also be said broadly to consist in the parts, elements and features referred to or indicated in the specification of the application, individually or collectively, in any or all combinations of two or more of said parts, elements or features. Where, in the foregoing description reference has been made to integers or components having known equivalents thereof, those integers are herein incorporated as if individually set forth.

Where any or all of the terms “comprise”, “comprises”, “comprised” or “comprising” are used in this specification (including the claims) they are to be interpreted as specifying the presence of the stated features, integers, steps or components, but not precluding the presence of one or more other features, integers, steps or components or group thereof.

It is to be understood that the following claims are provided by way of example only, and are not intended to limit the scope of what may be claimed in any future application. Features may be added to or omitted from the claims at a later date so as to further define or re-define the invention or inventions.

1. A system for providing respiratory support to a subject, the system including:
a flow source for providing a gas at a selected flow rate;
an invasive respiratory device for delivery of gases to an airway of the subject; and
a connector for coupling with the invasive respiratory device, the connector including a main body having:
a gases port for receiving a flow of gas from the flow source, wherein the gases port includes an inlet and an outlet;
an outlet port for outflow of gases from the main body; and
a device port couplable with the invasive respiratory device;
wherein the connector is configured to receive the flow of gas from the flow source via the inlet of the gases port, and to deliver a jet flow of gas through the outlet of the gases port,
wherein the system is configured to generate a pressure of at least about 2 cmH₂O about the device port when in use.

2. A system for providing respiratory support to a subject, the system including:
a flow source for providing a gas at a selected flow rate;
an invasive respiratory device for delivery of an airway of the subject; and
a connector for coupling with the invasive respiratory device, the connector including a main body having:
a gases port for receiving a flow of gas from the flow source, wherein the gases port includes an inlet and an outlet;
an outlet port for outflow of gases from the main body; and
a device port couplable with the invasive respiratory device;
wherein the connector is configured to receive the flow of gas from the flow source via the inlet of the gases port, and to deliver a jet flow of gas through the outlet of the gases port;
wherein a pressure loss between the device port and the outlet port of the connector is less than about 20 cmH₂O when in use.

3. A system for providing respiratory support to a subject, the system including:
a flow source for providing a gas at a selected flow rate;
an invasive respiratory device for delivery of gases to an airway of the subject; and
a connector for coupling with the invasive respiratory device, the connector including a main body having:
a gases port for receiving a flow of gas from the flow source, wherein the gases port includes an inlet and an outlet;
an outlet port for outflow of gases from the main body; and
a device port couplable with the invasive respiratory device;
wherein the connector is configured to receive the flow of gas from the flow source via the inlet of the gases port, and to deliver a jet flow of gas through the outlet of the gases port,
wherein a pressure loss between the outlet of the gases port and the outlet port of the connector is less than about 20 cmH₂O when in use.

4. (canceled)

5. The system according to claim 1, wherein the pressure about the device port is between about 2 cmH₂O and about 20 cmH₂O.

6. (canceled)

7. (canceled)

8-14. (canceled)

15. The system according to claim 1, wherein the selected flow rate is in a range of about 20 L/min to about 90 L/min.

16. The system according to claim 1, wherein the selected flow rate is in a range of about 0.5 L/min to about 25 L/min.

17. The system according to claim 1, further including a filter couplable with the outlet port of the connector for filtering the gases from the main body.

18-24. (canceled)

25. The system according to claim 1, further including a humidifier configured to condition the gas provided by the flow source to at least one of a selected temperature or a selected humidity.

26. The system according to claim 1, wherein the jet flow of gas delivered through the outlet of the gases port has a velocity in a range of about 5 m/s to about 60 m/s.

27. The system according to claim 1, wherein the outlet of the gases port has a hydraulic diameter in a range of about 2 mm to about 10 mm.

28. The system according to claim 27, wherein the hydraulic diameter is in a range of about 5 mm to about 8 mm.

29. The system according to claim 1, wherein a distance from the outlet of the gases port to a distal end portion of the invasive respiratory device when coupled to the device port is in a range of about 0 mm to about 60 mm.

30. The system according to claim 1, wherein the outlet of the gases port has a cross-sectional area in a range of about 10 mm² to about 60 mm².

31. The system according to claim 1, wherein:
a distance from the outlet of the gases port to a distal end portion of the invasive respiratory device when coupled to the device port is in a range of about 0 mm to about 60 mm;
the outlet of the gases port has a cross-sectional area in a range of about 10 mm² to about 60 mm²; and
a ratio of the cross-sectional area of the outlet of the gases port to the distance from the outlet of the gases port to the distal end portion of the invasive respiratory device is from about 1:1 to about 1:10.

32. The system according to claim 1, wherein the connector further includes an expiratory flow path between the device port and the outlet port, and wherein the expiratory flow path has a minimum cross-sectional area of at least about 25 mm².

33-36. (canceled)

37. The system according to claim 1, wherein the gases port further includes a flow constriction for providing the jet flow of gas through the outlet of the gases port.

38. The system according to claim 37, wherein the flow constriction is disposed between the inlet of the gases port and the device port.

39. The system according to claim 37, wherein the flow constriction includes a nozzle having the outlet of the gases port through which the jet flow of gas is delivered.

40. The system according to claim 37, wherein the flow constriction includes the outlet of the gases port having a plurality of apertures through which the jet flow of gas is delivered.

41. The system according to claim 37, wherein the flow constriction includes a tapered region for constricting the flow of gas prior to exiting the outlet.

42-82. (canceled)
